(19) United States
(12) Patent Application Publication
(10) Pub. No.: US 2022/0275329 A1
(43) Pub. Date: Sep. 1, 2022

(54) COMPOSITIONS AND METHODS FOR MATURING STEM CELL-DERIVED BETA CELLS

(71) Applicant: President and Fellows of Harvard College, Cambridge, MA (US)

(72) Inventors: Douglas A. Melton, Cambridge, MA (US); Aharon Helman, Cambridge, MA (US)

(21) Appl. No.: 17/616,993

(22) PCT Filed: Jun. 8, 2020
(86) PCT No.: PCT/US2020/036706

§ 371 Date: Dec. 6, 2021

Related U.S. Application Data
(60) Provisional application No. 62/858,301, filed on Jun. 6, 2019.

(51) Int. Cl. C12N 5/0735; C12N 5/071; C12N 5/077

(52) U.S. Cl. CPC C12N 5/0606; C12N 5/0676; C12N 5/0656; C12N 2501/727; C12N 2320/30

(57) ABSTRACT

Disclosed herein are methods for generating mature stem cell-derived beta cells and compositions including mature stem cell-derived beta cells.

### RELATED APPLICATIONS

This application claims the benefit of U.S. Provisional Application No. 62/858,301, filed on Jun. 6, 2019, the contents of which are hereby incorporated by reference in its entirety.

### BACKGROUND OF THE INVENTION

At birth, all mammals are faced with an abrupt challenge as they break from the supportive maternal environment into the outside world, a transition that requires an independent physiological system that includes feeding and breathing. Many systems, including the respiratory, circulatory, digestive and endocrine systems, undergo adaptations in their functional capacity during the early neonatal period, which is termed terminal or functional maturation (Morton and Brodsky, 2016; Ward Platt and Deshpande, 2005). Because nutrient consumption changes from relatively constant to pulsatile after birth, the metabolic adaptation of the neonate to intermittent feeding and glucose fluctuations is critical. Pancreatic beta cell maturation involves developing a higher threshold for glucose stimulation that results in inhibition of insulin secretion at low glucose and enhanced insulin secretion in response to high glucose-changes that are essential to maintain nearly constant glucose levels and avoid hypoglycemia (Blum et al., 2012; Rozzo et al., 2009). Further understanding of the functional maturation of cells and tissues is important for the application of stem cell-derived tissues in regenerative medicine (Robinton and Daley, 2012).

### SUMMARY OF THE INVENTION

There is a continued need for methods or protocols for the generation of mature SC-β cells that exhibit a GSIS response for use in cell therapy and screening, among other uses.

Disclosed herein are methods of producing a mature SC-β cell. The methods comprise contacting an immature or less mature β cell with an mTOR inhibitor, thereby producing a mature or more mature SC-β cell. In some embodiments the maturity of the β cell is demonstrated by its GSIS response, with more mature cells along a continuum evidencing an improved GSIS response compared to less mature cells. The improvement in the GSIS response can be qualitative or quantitative. Contacting a less mature β cell with an mTOR inhibitor, e.g., in vitro, as described herein results in a more mature β cell as evidenced by its GSIS response.

In some embodiments, the mTOR inhibitor is an inhibitor of both mTORC1 and mTORC2. In some embodiments, the mTOR inhibitor inhibits phosphorylation of Ribosomal protein S6. In some embodiments, the mTOR inhibitor inhibits both phosphorylation of Ribosomal protein S6 and 4E-BP1. In some embodiments, the mTOR inhibitor is selected from the group consisting of rapamycin, Torin1, Torin2, everolimus and temsirolimus.

In some embodiments, the immature β cell is derived from an iPS cell, an ES cell, and/or a fibroblast. In some embodiments, the mature SC-β cell exhibits increased GSIS response as compared to an immature β cell. In some embodiments, the immature β cell is contacted with the mTOR inhibitor during a final stage of a differentiation protocol. In some embodiments, the immature β cell is contacted with the mTOR inhibitor for a period of 1 to 3 days.

Also disclosed herein are methods of producing a mature SC-β cell. The methods comprise culturing an immature β cell in a nutrient poor culture medium, thereby producing a mature SC-β cell.

In some embodiments, the nutrient poor culture medium comprises a reduced level of amino acids as compared to a non-responsive culture medium comprising 100% amino acids. In some embodiments, the nutrient poor culture medium comprises 75% amino acid levels as compared to the non-responsive culture medium. In some embodiments, the SC-β cells cultured in a culture medium comprising 75% amino acid levels exhibit at least a 1.1 fold increase in GSIS response. In some embodiments, the nutrient poor culture medium comprises 50% amino acid levels as compared to the non-responsive culture medium. In some embodiments, the SC-β cells cultured in a culture medium comprising 50% amino acid levels exhibit at least a 1.6 fold increase in GSIS response. In some embodiments, the nutrient poor culture medium comprises 25% amino acid levels as compared to the non-responsive culture medium. In some embodiments, the SC-β cells cultured in a culture medium comprising 25% amino acid levels exhibit a 2 fold increase in GSIS response.

In some embodiments, the immature β cell is derived from an iPS cell, an ES cell, and/or a fibroblast.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1H demonstrates a dynamic response of mTORC1 to nutrients in mature islet cells. FIG. 1A provides representative p-S6 staining histograms of c-peptide+ cells (human beta cells, left column) and glp2+ cells (human alpha cells, right column) following 30 minute incubation of human islets in RPMI with indicated nutrients. Note the full response of human beta cells only in the presence of glucose as opposed to alpha cells that require only amino acids. FIG. 1B provides p-S6 staining histograms of c-peptide+ cells (human beta cells) following 30 minute incubation of human islets in RPMI with nutrients, insulin or insulin receptor antagonist (s961). FIG. 1C shows percentages of p-S6 positive and negative beta (left, blue) and alpha (right, red) cells in human islets, after 30 minute incubation in the indicated conditions (ex4, exendin-4; fsk, forskolin; leu, leucine; dz, diazoxide), detected by FACS compared to secondary antibodies only control. For beta cells, n=10, 10, 10, 10, 2, 2, 4, 4, 3, 4, 2, 4; bottom to top. For alpha cells n=10 for each condition. In each experiment, at least 10k islets from a single donor were used. FIG. 1D provides representative p-S6 staining histograms of insulin+ cells (mouse beta cells, left column) and glucagon+ cells (mouse alpha cells, right column) following 30 minute incubation of mouse islets in RPMI with indicated nutrients. Note the full response of beta cells only in the presence of glucose as opposed to alpha cells that require only amino acids. FIG. 1E provides p-S6 staining histograms of insulin+ cells following 30 minute incubation of mouse islets in RPMI with indicated nutrients, glp1 analog (exendin-4, ex4) and potassium channel activator (diazoxide, dz). FIG. 1F shows percentages of p-S6 positive and negative beta (left, blue) and alpha (right, red) cells in mouse islets, after 30 minute incubation in the indicated conditions, detected by FACS compared to secondary antibodies only control. For beta cells, n=10, 7, 6, 10, 4, 4, 3, 2, 2, 2; bottom to top. For alpha cells n=5, 4, 2, 5; bottom to top. In each experiment, islets from at least 5 mice were used. FIG. 1G provides representative immunostainings of p-S6 (green), glucagon (red) and insulin (labeling beta cells, blue), in pancreatic islets of fasted mice injected with the indicated nutrients or re-fed. Bottom images are presented without insulin staining. Experiment was repeated twice, n=2 for each condition. FIG. 1H provides a schematic illustration of the dynamic control of mTORC1 activity by nutrients in beta (blue line) and alpha (red line) cells under fast and fed conditions.

FIGS. 2A-2H demonstrate glucose-independent activity of mTORC1 in immature islet cells. FIG. 2A provides representative immunostainings of p-S6 (green), glucagon (red) and insulin (labeling beta cells, blue), in pancreatic islet of a fasted pregnant female mouse (left panels) and its E18 embryo (right panels). Bottom images are presented without insulin staining. Note the strong mTORC1 activity in fetal compared to maternal beta cells. Experiment was repeated twice, in each experiment pancreases of the embryos were combined and stained together. FIG. 2B provides representative immunostainings of p-S6 (green) and insulin (labeling beta cells, blue), in adult (left panels) and fetal (right panels) pancreatic islets. Bottom images are presented without insulin staining. For each group n=2. FIG. 2C provides representative p-S6 staining histograms of c-peptide+ fetal (left column) and adult (right column) cells following 30 minute incubation of islets in RPMI with indicated nutrients. Note the strong S6 phosphorylation in fetal human beta cells in response to amino acids only. FIG. 2D shows percentages of p-S6 positive and negative beta cells in fetal (left) and adult (right) human islets, detected by FACS analysis compared to secondary antibodies only control. Experiment was repeated twice on two different samples of fetal pancreas. FIG. 2E provides representative p-S6 staining histograms of insulin+ cells from mice of indicated age following 30 minutes incubation of islets in RPMI with indicated nutrients. Note that at P7 mTORC1 dynamics is equivalent to mature beta cells. FIG. 2F shows percentages of p-S6 positive beta cells from mice of indicated age in response to indicated nutrients, detected by FACS analysis compared to secondary antibodies only control. Experiment was repeated twice, n=2, 3, 3, 4; left to right. FIG. 2G provides representative immunostainings of p-S6 (green), glucagon (red), and insulin (labeling beta cells, blue), in pancreatic islets of fasted P1 (left panels) and P5 (right panels) neonatal mice, injected with saline or glucose as indicated. Bottom images are presented without insulin staining. Note that mTORC1 response to glucose is acquired at postnatal day 5. Experiments were repeated twice. In each experiment, at least 5 neonatal pancreatas were combined and stained together. FIG. 2H shows a relative ratio of amino acids and their metabolites (left graph) and glucose (right graph) in the serum of mice of indicated age. Experiment was repeated twice, n=4, 15, 16, 4; left to right, in each graph.

FIGS. 3A-3G demonstrate mTORC1 activity inversely correlates with SC-beta cell function. FIG. 3A provides a schematic illustration showing a comparison between functional and dysfunctional SC-derived beta cells. SC-beta cells were stained and sorted using TSQ (Zn binding dye) and used for gene expression profiling. FIG. 3B shows insulin levels secreted by SC-beta cells of indicated differentiation protocol after incubation in low (2.8 mM) and high (16.7 mM) glucose concentrations for 1 h. Secreted insulin levels were normalized to cell number of each sample. Experiment was repeated on three differentiation flasks, three replicates for each flask. FIG. 3C shows enrichment significance of gene sets associated with SC-beta cells dysfunction among genes upregulated in the non-responsive condition. Values indicate −Log 10 P, hypergeometric test. RNAseq was conducted on three independent differentiation flasks for each condition. FIG. 3D shows insulin levels secreted by SC-beta cells from indicated differentiation protocol in a dynamic perfusion assay in low (2.8 mM, grey background) and high (16.7 mM, purple background) glucose concentrations. Secreted insulin levels were normalized to basal insulin secretion of each sample. Experiment was done on three independent differentiation flasks for each treatment. FIG. 3E provides representative p-S6 FACS staining histograms of c-peptide+ cells (SC-beta cells) following incubation of SC-clusters in rich or poor RPMI media (red and blue histograms, respectively) and in rich media with Torin1 (green histogram). FIG. 3F provides quantification of p-S6 intensity from indicated conditions in three independent experiments. FIG. 3G shows insulin levels secreted by SC-beta cells that were grown in indicated conditions and incubated in low (2.8 mM) and high (16.7 mM) glucose concentrations for 1 h. Secreted insulin levels were normalized to insulin content of each sample. Lines indicate individual differentiation flask. SI indicates stimulation index.

FIGS. 4A-4F demonstrate a shift in mTORC1 nutrient sensitivity of SC-beta cells induced by transplantation. FIG. 4A provides representative p-S6 staining histograms of c-peptide+ cells (SC-beta cells) following 30 minute incubation of SC-clusters in RPMI with indicated nutrients. Note the strong mTORC1 response of SC-beta cells to amino acids only. FIG. 4B shows percentages of p-S6 positive and negative SC-beta cells detected by FACS analysis compared to secondary antibodies only control. Experiment was repeated in nine differentiation flasks. FIG. 4C provides representative immunostainings of p-S6 (green) and insulin (labeling SC-beta cells, blue), in clusters of in-vitro differentiated stem cells incubated with indicated nutrients. Bottom images are presented without insulin staining. Note the strong mTORC1 response of SC-beta cells to amino acids only. Experiment was repeated twice using independent differentiation flasks. FIG. 4D provides representative immunostainings of p-S6 (green) and insulin (labeling SC-beta cells, blue), in kidney capsule-transplants of SC-beta cells. Transplanted mice were fasted overnight and injected with the indicated nutrients or re-fed. Bottom images are presented without insulin staining. Note the weak response of mTORC1 to leucine and the acquired glucose responsiveness after transplantation. For each condition n=2. FIG. 4E provides a gene expression heat map of known regulators of mTORC1 signaling in SC-beta cells before and after transplantation. Note that SESN1 and SESN2 are significantly upregulated in SC-beta cells after transplantation. SC-beta cells were sorted from five independent differentiation flasks and from seven transplanted mice. FIG. 4F shows expression levels of SESN and rpS6K genes in SC-beta (black) and transplanted SC-beta (grey) TSQ-sorted cells.

FIGS. 5A-5C demonstrate Sestrins control mTORC1 dynamics and insulin secretion in mature beta cells. FIG. 5A provides representative p-S6 staining histograms of insulin+ cells of wt (left column) and SESN1 and SESN2 deficient (right column) mice following 30 minutes incubation of mouse islets in RPMI with indicated nutrients. Note that depletion of leucine strongly inhibited mTORC1 signaling in wt but not in SESN-deficient beta cells. FIG. 5B shows percentages of p-S6 positive and negative wt (left) and SESN knockout (right) beta cells in response to indicated nutrients, detected by FACS analysis compared to secondary antibodies only control. Experiment was repeated twice. In each experiment islets from at least three mice were used for each group. FIG. 5C shows insulin levels secreted by isolated islets from wt (left) and SESN2 knockout (middle) and SESN1 and SESN2 double knockout (left) beta cells, in a static GSIS assay in low (2.8 mM, black), high (16.7 mM, light grey) glucose concentrations and KCl (30 mM, dark grey). Secreted insulin levels were normalized to insulin content of each sample. Experiment was repeated twice. In each experiment islets from at least three mice were used for each group.

FIGS. 6A-6F demonstrate nutrient availability dictates SC-beta cell function. FIG. 6A provides representative p-S6 staining histograms of c-peptide+ cells (SC-beta cells) and percentages of p-S6 positive and negative SC-beta cells grown in media with indicated amino acid levels compared to the non-responsive culture media (100% amino acids). FIG. 6B provides representative immunostainings of p-S6 (green), and insulin (labeling SC-beta cells, blue), in clusters of in-vitro differentiated stem cells incubated with indicated nutrients. Note the strong phosphorylation of S6 in response to amino acids only. Bottom images are presented without insulin staining. FIG. 6C provides representative immunostainings of p-S6 (green), and insulin (labeling SC-beta cells, blue), in clusters of in-vitro differentiated stem cells incubated with indicated nutrients. Note the weak phosphorylation of S6 in SC-beta cells in response to amino acids only. Bottom images are presented without insulin staining. FIG. 6D shows insulin levels secreted by SC-beta cells from indicated growing condition in a dynamic perfusion assay in low (2.8 mM, grey background), high (16.7 mM, purple background) glucose concentrations and KCl (30 mM, orange background). Secreted insulin levels were normalized to basal insulin secretion of each sample. FIG. 6E shows basal and stimulated insulin secretion levels as measured by the area under the curve (AUC) of the dynamic insulin secretion in FIG. 6D, for the indicated conditions. FIG. 6F provides representative c-peptide staining histograms (left column) and bar graph (right column) of c-peptide+ cells (SC-beta cells) grown in media with indicated amino acid levels compared to non-responsive culture media (100% amino acids). The experiments described in FIGS. 6A-6F were repeated twice using three independent differentiation flasks in each experiment.

FIG. 7 shows mTOR activation from an immature metabolic state to a mature metabolic state.

FIGS. 8A-8F demonstrate a dynamic response of mTORC1 to nutrients. FIG. 8A provides analysis strategy to detect mTORC1 activation in beta and alpha cells. Clusters are dispersed, fixed and stained for C-peptide, glucagon and p-S6, and the intensity of p-S6 and the percentages of p-S6+ cells in C-peptide+Glucagon− cells in the different conditions is calculated. FIG. 8B provides representative p-S6 staining histograms of c-peptide+ cells (human beta cells) following 30 minutes incubation of human islets in RPMI with indicated nutrients. Note that phosphorylation of S6 in response to glucose stimulation is inhibited by Torin1 and is not mimicked by addition of insulin. FIG. 8C provides representative p-S6 staining histograms of c-peptide+ cells (human beta cells, left column) and glp2+ cells (human alpha cells, right column) following 30 minutes incubation of human islets in RPMI with indicated nutrients. Both beta and alpha cells show greater dependence on leucine than arginine. FIG. 8D provides representative p-S6 staining histograms of c-peptide+ cells (human beta cells) following 30 minutes incubation of human islets in RPMI with glucose in the indicated concentrations and with 1 mM leucine. Note that addition of leucine amplifies the response to glucose in beta cells. FIG. 8E provides representative p-S6 staining histograms of c-peptide+ cells (human beta cells) following 30 minutes incubation of human islets in RPMI with glucose in the indicated concentrations and with 1 mM leucine. Note that amplifiers of insulin secretion (forskolin, fsk and exendin4, ex4) strongly activate mTORC1 in beta cells while blocking insulin secretion (with diazoxide, dz) inhibits mTORC1 activation. FIG. 8F provides p-S6 staining histograms of insulin+ cells (mouse beta cells) following 30 minutes incubation of mouse islets in RPMI with indicated nutrients. Insulin and insulin receptor antagonist (s961) do not affect mTORC1 activation in beta cells.

FIGS. 9A-9D demonstrate glucose-independent activity of mTORC1. FIG. 9A provides representative p-S6 staining histograms of insulin+ cells from mice of indicated age following 30 minute incubation of islets in RPMI with glucose and amino acids with or without diazoxide (dz). Note that diazoxide inhibits mTORC1 activation only at P5 similarly to mature beta cells. FIG. 9B provides representative p-S6 staining histograms of glucagon+ cells from mice of indicated age following 30 minutes incubation of islets in RPMI with indicated nutrients. Note that at P7 rnTORC1 dynamics is equivalent to mature alpha cells. FIG. 9C shows percentages of p-S6 positive alpha cells from mice of indicated age in response to indicated nutrients, detected by FACS analysis compared to secondary antibodies only control. FIG. 9D provides a representative heat map of metabolite abundance in serum of embryos, neonates and adult mice.

FIGS. 10A-10D demonstrate mTORC1 activity inversely correlates with SC-beta cell function. FIG. 10A shows sorting strategy for SC-beta cells using TSQ, a Zn-binding dye. Note that sorted TSQ+ cells are enriched for c-peptide+ and c-peptide+nkx6.1+ cells compared to TSQ− cells (77% and 42% compared to 8.5% and 3%, respectively). FIG. 10B provides a heat map of differentially expressed genes between functional and dysfunctional TSQ+ SC-beta cells. FIG. 10C shows mRNA expression of mTORC1 regulators in v4 and v8 TSQ+ SC-bet cells. FIG. 10D shows analysis strategy to detect mTORC1 activation in SC-beta cells. Clusters are dispersed, fixed and stained for C-peptide, glucagon and p-S6, and the intensity of p-S6 and the percentages of p-S6+ cells in C-peptide+Glucagon− cells in the different conditions is calculated.

FIG. 11 provides a gene expression heat map of known regulators of beta cell maturation and function. SC-beta cells were sorted from five independent differentiation flasks and from seven transplanted mice.

FIGS. 12A-12C demonstrate Sestrins control mTORC1 dynamics and insulin secretion. FIG. 12A provides representative p-S6 staining histograms of glucagon+ cells of wt (left column) and SESN2 deficient (right column) mice following 30 minutes incubation of mouse islets in RPMI with indicated nutrients. Note that depletion of leucine strongly inhibited mTORC1 signaling in wt but not in SESN2-deficient alpha cells. FIG. 12B shows percentages of p-S6 positive and negative wt (left) and SESN2 knockout (right) alpha cells in response to indicated nutrients, detected by FACS analysis compared to secondary antibodies only control. Experiment was repeated twice. In each experiment islets from at least three mice were used for each group. FIG. 12C provides insulin levels secreted by wt (blue) or SESN deficient (red) mouse islets in a dynamic perfusion assay in low (2.8 mM, grey background) and high (16.7 mM, purple background) glucose concentrations. Secreted insulin levels were normalized to basal insulin secretion of each sample. Experiment was done on three mice from each group.

### DETAILED DESCRIPTION OF THE INVENTION

Aspects of the disclosure relate to compositions, methods, kits, and agents for generating mature stem cell-derived beta cells (referred to herein as non-naturally occurring beta cells, non-native beta cells, or mature beta cells) from at least one stem cell, and mature beta cells produced by those compositions, methods, kits, and agents for use in cell therapies, assays, and various methods of treatment.

The in vitro-produced beta cells generated according to the methods described herein demonstrate many advantages; for example, they are more mature (e.g., exhibit an improved GSIS response) compared with the beta cell prior to contacting with the agent. In addition, the generated beta cells may provide a new platform for screening, cell therapy (e.g., transplantation into a subject in need of additional and/or functional beta cells) and research.

Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell leads to the pancreatic pathway, where ˜98% of the cells become exocrine, ductular, or matrix cells, and ˜2% become endocrine cells.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell type: internal organs, skin, bones, blood, and connective tissue are all made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro. Unless otherwise indicated the methods described herein can be performed both in vivo and in vitro.

The term “exocrine cell” as used herein refers to a cell of an exocrine gland, i.e. a gland that discharges its secretion via a duct. In particular embodiments, an exocrine cell refers to a pancreatic exocrine cell, which is a pancreatic cell that produces enzymes that are secreted into the small intestine. These enzymes help digest food as it passes through the gastrointestinal tract. Pancreatic exocrine cells are also known as islets of Langerhans that secrete two hormones, insulin and glucagon.

As used herein, the term “adult cell” refers to a cell found throughout the body after embryonic development.

The term “endoderm cell” as used herein refers to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of respiratory and digestive tracts (e.g. the intestine), the liver and the pancreas.

The term “a cell of endoderm origin” as used herein refers to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) develop from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates.

The term “pancreatic progenitor” or “pancreatic precursor” are used interchangeably herein and refer to a stem cell which is capable of forming any of; pancreatic endocrine cells, pancreatic exocrine cells, or pancreatic duct cells. The term “pdx1-positive pancreatic progenitor” as used herein refers to a cell which is a pancreatic endoderm (PE) cell. A Pdx1-positive pancreatic progenitor expresses the marker Pdx1. Other markers include, but are not limited to Cdcp1, or Ptf1a, or HNF6 or NRx2.2. The expression of Pdx1 may be assessed by any method known by the skilled person such as immunochemistry using an anti-Pdx1 antibody or quantitative RT-PCR. The term “pdx1-positive, NKX6-1-positive pancreatic progenitor” as used herein refers to a cell which is a pancreatic endoderm (PE) cell. A pdx1-positive, NKX6-1-positive pancreatic progenitor expresses the markers Pdx1 and NKX6-1. Other markers include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR.

The terms “mature stem cell-derived beta cell”, “SC-beta cell”, and “mature SC-beta cell” refer to cells (e.g., pancreatic β cells) that display at least one marker indicative of a pancreatic β cell, express insulin, and display a GSIS response characteristic of an endogenous mature β cell. In some embodiments, the “SC-β cell” comprises a mature pancreatic β cells. It is to be understood that the SC-β cell need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the invention is not intended to be limited in this manner).

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “phenotype” refers to one or a number of total biological characteristics that define the cell or organism under a particular set of environmental conditions and factors, regardless of the actual genotype.

The term “pluripotent” as used herein refers to a cell with the capacity to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refer to a pluripotent stem cell artificially derived (e.g., induced or by complete reversal) from a non-pluripotent cell, typically an adult somatic cell, for example, by inducing a forced expression of one or more genes.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

As used herein, the term “proliferation” means growth and division of cells. In some embodiments, the term “proliferation” as used herein in reference to cells refers to a group of cells that can increase in number over a period of time.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as a beta cell precursors), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting at least one immature beta cell or a precursor thereof with a maturation factor, or combination of maturation factors) is intended to include incubating the differentiation medium and/or agent and the cell together in vitro (e.g., adding the maturation factors to cells in culture). In some embodiments, the term “contacting” is not intended to include the in vivo exposure of cells to the compounds as disclosed herein that may occur naturally in a subject (i.e., exposure that may occur as a result of a natural physiological process). The step of contacting at least one immature beta cell or a precursor thereof with a maturation factor as in the embodiments described herein can be conducted in any suitable manner. For example, the cells may be treated in adherent culture, or in suspension culture. The disclosure contemplates any conditions which promote the formation of mature stem cell-derived beta cells. Examples of conditions that promote the formation of mature stem cell-derived beta cells include, without limitation, suspension culture in low attachment tissue culture plates, spinner flasks, aggrewell plates. In some embodiments, the inventors have observed that mature stem cell-derived beta cells have remained stable in media containing 20% serum (e.g., heat inactivated fetal bovine serum).

It is understood that the cells contacted with a maturation factor can also be simultaneously or subsequently contacted with another agent, such as a growth factor or other differentiation agent or environments to stabilize the cells, or to differentiate the cells further.

Similarly, at least one immature beta cell or a precursor thereof can be contacted with at least one maturation factor and then contacted with at least another maturation factor. In some embodiments, the cell is contacted with at least one maturation factor, and the contact is temporally separated, and in some embodiments, a cell is contacted with at least one maturation factor substantially simultaneously. In some embodiments, the cell is contacted with at least two, at least three, at least four, at least five, at least six, at least seven, at least eight, at least nine, or at least 10 maturation factors.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a mature beta cell described herein.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification—e.g., in a rabbit reticulocyte in vitro translation assay), the modification is not suitable for the methods and compositions described herein.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. MoI. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL world-wide web address of: “ncbi.nlm nih.gov” for these programs. In a specific embodiment, percent identity is calculated using BLAST2 with default parameters as provided by the NCBI.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of stem cell-derived beta cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not stem cell-derived beta cells as defined by the terms herein. In some embodiments, the present invention encompasses methods to expand a population of stem cell-derived beta cells, wherein the expanded population of stem cell-derived beta cells is a substantially pure population of stem cell-derived beta cells.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “lineages” as used herein describes a cell with a common ancestry or cells with a common developmental fate. For example, in the context of a cell that is of endoderm origin or is “endodermal linage” this means the cell was derived from an endoderm cell and can differentiate along the endoderm lineage restricted pathways, such as one or more developmental lineage pathways which give rise to definitive endoderm cells, which in turn can differentiate into liver cells, thymus, pancreas, lung and intestine.

As used herein, the term “xenogeneic” refers to cells that are derived from different species.

As used herein, the term “DNA” is defined as deoxyribonucleic acid.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate or dedifferentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene. In some cases, expression of the reporter gene may be determined in live cells. Where live cell reporter gene assays are used, reporter gene expression may be monitored at multiple time points, e.g., 2, 3, 4, 5, 6, 8, or 10 or more time points. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms “treat”, “treating”, “treatment”, etc. refer to providing medical or surgical attention, care, or management to an individual. The individual is usually ill or injured, or at increased risk of becoming ill relative to an average member of the population and in need of such attention, care, or management. It may include administering to a subject an effective amount of a composition so that the subject exhibits a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. The term “treatment” includes prophylaxis. Those in need of treatment include those already diagnosed with a condition (e.g., muscle disorder or disease), as well as those likely to develop a condition due to genetic susceptibility or other factors.

As used herein, the terms “administering,” “introducing” and “transplanting” are used interchangeably in the context of the placement of cells of the invention into a subject, by a method or route which results in at least partial localization of the introduced cells at a desired site. The cells can be implanted directly to the pancreas or gastrointestinal tract, or alternatively be administered by any appropriate route which results in delivery to a desired location in the subject where at least a portion of the implanted cells or components of the cells remain viable. The period of viability of the cells after administration to a subject can be as short as a few hours, e.g. twenty-four hours, to a few days, to as long as several years. In some instances, the cells can also be administered subcutaneously, for example, in a capsule (e.g., microcapsule) to maintain the implanted cells at the implant location and avoid migration of the implanted cells.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and infrasternal injection and infusion. The phrases “systemic administration,” “administered systemically”, “peripheral administration” and “administered peripherally” as used herein mean the administration of stem cell-derived cells and/or their progeny and/or compound and/or other material other than directly into the central nervous system, such that it enters the animal's system and, thus, is subject to metabolism and other like processes, for example, subcutaneous administration.

The term “tissue” refers to a group or layer of specialized cells which together perform certain special functions. The term “tissue-specific” refers to a source of cells from a specific tissue.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold, or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Stem Cells

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells for producing beta cells (e.g., mature beta cells) or precursors thereof, germ cells may be used in place of, or with, the stem cells to provide at least one beta cell, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (stemcells.nih.gov/info/scireport/2006report.htm, 2006). One possible application of ES cells is to generate new pancreatic beta cells for the cell replacement therapy of type I diabetics, by first producing endoderm, e.g., definitive endoderm, from, e.g., hESCs, and then further differentiating the definitive endoderm into at least one insulin-positive endocrine cell or precursor thereof, then further differentiating the at least one insulin-positive endocrine cell or precursor thereof into an immature stem cell-derived beta cell, and then further differentiating or maturing the at least one immature stem cell-derived beta cell or precursor thereof into a mature stem cell-derived beta cell.

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1: Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (˜200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be used be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which is incorporated herein in its entirety by reference.

Briefly, genital ridges processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10 μM forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad γ-irradiation ˜0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g. a cell not committed to a specific linage) prior to exposure to at least one maturation factor according to the methods as disclosed herein, whereas in other examples it may be desirable to differentiate the stem cells to one or more intermediate cell types prior to exposure of the at least one maturation factor (s) described herein. For example, the stems cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature stem cell-derived beta cells did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subjects adipose tissue, for example using the CELUTION™ SYSTEM from Cytori, as disclosed in U.S. Pat. Nos. 7,390,484 and 7,429,488 which is incorporated herein in its entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497; Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic micro-environment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

Cloning and Cell Culture

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Cabs eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM β-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Alternatively, pluripotent SCs can be maintained in an undifferentiated state even without feeder cells. The environment for feeder-free cultures includes a suitable culture substrate, particularly an extracellular matrix such as MATRIGEL® (gelatinous protein mixture) or laminin. Typically, enzymatic digestion is halted before cells become completely dispersed (˜5 min with collagenase IV). Clumps of ˜10 to 2,000 cells are then plated directly onto the substrate without further dispersal.

Generating Mature Stein Cell-Derived Beta Cells

Aspects of the disclosure relate to generating mature stem cell-derived beta cells. Generally, mature stem cell-derived beta cells or precursors thereof, e.g., immature beta cells produced according to the methods disclosed herein demonstrate several hallmarks of functional mature beta cells, including, but not limited to, exhibiting a GSIS response.

The mature stem cell-derived beta cells can be produced according to any suitable culturing protocol or series of culturing protocols to differentiate a stem cell or pluripotent cell to a desired stage of differentiation. In some embodiments, the mature stem cell-derived beta cells or the precursors thereof are produced by culturing at least one pluripotent cell for a period of time and under conditions suitable for the at least one pluripotent cell to differentiate into the mature stem cell-derived beta cells or the precursors thereof.

In some embodiments, the mature stem cell-derived beta cells or precursors thereof are maintained in culture by methods known by one of ordinary skill in the art, and in some embodiments, propagated prior to being converted into mature stem cell-derived beta cells by the methods as disclosed herein.

Further, mature stem cell-derived beta cells or precursors thereof, e.g., immature beta cells, can be from any mammalian species, with non-limiting examples including a murine, bovine, simian, porcine, equine, ovine, or human cell. For clarity and simplicity, the description of the methods herein refers to a mammalian mature stem cell-derived beta cell or precursor thereof, but it should be understood that all of the methods described herein can be readily applied to other cell types of mature stem cell-derived beta cells or precursors thereof. In some embodiments, the mature stem cell-derived beta cells or precursors thereof are derived from a human individual.

Where the immature beta cells or precursors thereof are maintained under in vitro conditions, conventional tissue culture conditions and methods can be used, and are known to those of skill in the art. Isolation and culture methods for various cells are well within the abilities of one skilled in the art.

In the methods of the disclosure at least one mature stem cell-derived beta cell or a precursor thereof can, in general, be cultured under standard conditions of temperature, pH, and other environmental conditions, e.g., as adherent cells in tissue culture plates at 37° C. in an atmosphere containing 5-10% C0₂. The cells and/or the culture medium are appropriately modified to achieve conversion to mature stem cell-derived beta cells as described herein.

In certain examples, the maturation factors can be used to induce the differentiation of at least one immature beta cell or precursor thereof by exposing or contacting at least one immature beta cell or precursor thereof with an effective amount of a maturation factor described herein to differentiate the at least one immature beta cell or precursor thereof into at least one mature stem cell-derived beta cell.

Accordingly, included herein are cells and compositions made by the methods described herein. The exact amount and type of maturation factor can vary depending on the number of immature beta cells or precursors thereof, the desired differentiation stage and the number of prior differentiation stages that have been performed.

In certain examples, a maturation factor is present in an effective amount. As used herein, “effective amount” refers to the amount of the compound that should be present for the differentiation of at least 10% or at least 20% or at least 30% of the cells in a population of immature beta cells or precursors thereof into mature stem cell-derived beta cells.

In additional examples, maturation factors can be present in the culture medium of the at least one immature beta cell or precursor thereof, or alternatively, the maturation factors may be added to the at least one immature beta cell or precursor thereof during some stage of growth.

Where the at least one immature beta cell or a precursor thereof is maintained under in vitro conditions, conventional tissue culture conditions and methods can be used, and are known to those of skill in the art. Isolation and culture methods for various cells are well within the abilities of one skilled in the art.

Aspects of the disclosure involve mature stem cell-derived beta cells. Mature stem cell-derived beta cells of use herein can be derived from any source or generated in accordance with any suitable protocol. hi some aspects, immature beta cells are induced to mature into mature stem cell-derived beta cells.

In some embodiments mature stem cell-derived beta cells may be produced using the methods disclosed in WO 2015/002724 and WO 2014/201167, both of which are incorporated herein by reference. In some embodiments the methods disclosed in WO 2015/002724 and WO 2014/201167 are altered or modified (e.g., at Stage 6). In some embodiments the mature stem cell-derived beta cells are produced using the protocols identified as v4 or v8, and detailed in Table 1.

TABLE 1
Differentiation Protocols

| Protocol version | | v4 | v8 |
|---|---|---|---|
| Stage 1 | Duration base media | 3 days | 3 days |
| | | S1 | S1 |
| | Factors | Activin A | Activin A |
| | | CHIR99021 | CHIR99021 |
| Stage 2 | Duration base media | 3 days | 3 days |
| | | S2 | S2 |
| | Factors | KGF | KGF |
| Stage 3 | Duration base media | 2 days | 2 days |
| | | S3 | S3 |
| | Factors | RA | RA |
| | | KGF | KGF |
| | | SANT1 | SANT1 |
| | | LDN193189 | LDN193189 |
| | | PdBU | PdBU |
| | | Y27632 | Y27632 |
| Stage 4 | Duration base media | 5 days | 5 days |
| | | S3 | S3 |
| | Factors | KGF | KGF |
| | | SANT1 | SANT1 |
| | | RA | RA |
| | | Y27632 | Y27632 |
| | | Activin A | Activin A |
| Stage 5 | Duration base media | 7 days | 7 days |
| | | BE5 | BE5 |
| | Factors | XXI | XXI |
| | | Alk5i | Alk5i |
| | | T3 | T3 |
| | | RA | RA |
| | | SANT1 | SANT1 |
| | | Betacellulin | Betacellulin |
| Stage 6 | Duration base media | CMRLS | S3 |
| | | (+10% FBS) | |
| | Factors | Alk5i | none |
| | | T3 | |

In some embodiments, the disclosure provides a method for generating mature stem cell-derived beta cells from immature beta cells (e.g., immature stem cell-derived beta cells), the method comprising culturing immature stem cell-derived beta cells obtained at Stage 6 of a differentiation protocol in a nutrient poor culture medium to induce the maturation (e.g., in vitro maturation) of at least one immature beta cell in the population into a mature stem cell-derived beta cell. A nutrient poor culture medium may also be referred to herein as a responsive culture medium. In some embodiments, the nutrient poor culture medium comprises a reduced level of amino acids as compared to a culture medium comprising 100% amino acids (also referred to herein as a non-responsive culture medium). For example, a nutrient poor culture medium may have 75%, 50%, or 25% of the amino acid levels of the non-responsive culture medium. In some embodiments, a nutrient poor culture medium comprises 75% of the amino acid levels of the non-responsive culture medium. In some embodiments, a nutrient poor culture medium comprises 50% of the amino acid levels of the non-responsive culture medium. In some embodiments, a nutrient poor culture medium comprises 25% of the amino acid levels of the non-responsive culture medium.

In some embodiments, the disclosure provides a method for generating mature stem cell-derived beta cells from immature beta cells (e.g., immature stem cell-derived beta cells), the method comprising contacting a population of cells comprising immature beta cells with at least one maturation factor comprising an mTOR inhibitor, to induce the maturation (e.g., in vitro maturation) of at least one immature beta cell in the population into a mature stem cell-derived beta cell. In some embodiments, a population of cells comprising immature beta cells is contacted with at least one maturation factor (e.g., mTOR inhibitor, PI3K inhibitor, or Akt inhibitor). In some aspects, the PI3K/Akt/mTOR pathway is manipulated (e.g., inhibited) to enhance the maturation of beta cells derived from stem cells.

The disclosure contemplates the use of any mTOR inhibitor that encourages immature beta cells (e.g., immature stem cell-derived beta cells) to differentiate and/or mature into mature stem cell-derived beta cells (e.g., alone or in combination with another maturation factor). In some embodiments, mTOR comprises mTORC1 and/or mTORC2. In some embodiments, the mTOR inhibitor is an inhibitor of mTORC1 and/or mTORC2. In some embodiments, the mTOR inhibitor inhibits phosphorylation of 4E-BP1. Inhibiting phosphorylation of 4E-BP1 may affect regulation of the oxidative phosphorylation pathway. Non-limiting examples of modulators of the oxidative phosphorylation pathway include 4EGI-1, JR-AB2-011 (an mTORC2 inhibitor), AICAR (an AMPK activator), metformin (an AMPK activator and mTORC1/2 inhibitor), and HLM006474 (an E2F inhibitor). In some embodiments, the mTOR inhibitor inhibits phosphorylation of 4E-BP1 and Ribosomal protein S6. In some embodiments, the mTOR inhibitor comprises Torin1, Torin2, rapamycin, everolimus, and/or temsirolimus. In certain embodiments, a population of cells comprising immature beta cells is contacted with Torin1, to induce the maturation of at least one immature beta cell in the population into a mature stem cell-derived beta cell. In some embodiments, a population of cells comprising immature beta cells is contacted with Torin2, to induce the maturation of at least one immature beta cell in the population into a mature stem cell-derived beta cell.

Aspects of the disclosure involve generating mature stem cell-derived beta cells which exhibit a GSIS response. In some embodiments, the GSIS response of the mature stem cell-derived beta cells is increased by at least 1.0 fold, 1.1 fold, 1.2 fold, 1.3 fold, 1.4 fold, 1.5 fold, 1.6 fold, 1.7 fold, 1.8 fold, 1.9 fold, or 2.0 fold as compared to the GSIS response of immature beta cells.

Aspects of the disclosure involve generating mature stem cell-derived beta cells which resemble endogenous mature pancreatic beta cells in form and function, but nevertheless are distinct from native mature pancreatic beta cells.

Generating mature stem cell-derived beta cells by conversion or maturation of at least one immature beta cell or a precursor thereof using the methods of the disclosure has a number of advantages. First, the methods of the disclosure allow one to generate autologous stem cell-derived beta cells, which are cell specific to and genetically matched with an individual. In general, autologous cells are less likely than non-autologous cells to be subject to immunological rejection. The cells are derived from at least one immature beta cell or a precursor thereof, e.g., an insulin-positive endocrine cell obtained by reprogramming a somatic cell (e.g., a fibroblast) from the individual to an induced pluripotent state, and then culturing the pluripotent cells to differentiate at least some of the pluripotent cells to at least one immature beta cell or precursor, followed by the induced maturation in vitro of the at least one immature beta cell into a mature stem cell-derived beta cell.

In some embodiments, a subject from which at least one immature beta cell or precursor thereof are obtained is a mammalian subject, such as a human subject. In some embodiments, the subject is suffering from diabetes. In such embodiments, the at least one immature beta cell or precursor thereof can be differentiated into a mature stem cell-derived beta cell ex vivo by the methods as described herein and then administered to the subject from which the cells were harvested in a method to treat the subject for diabetes.

In some embodiments, the mature stem cell-derived beta cells are a substantially pure population of mature stem cell-derived beta cells. In some embodiments, a population of mature stem cell-derived beta cells or precursors thereof comprises a mixture of pluripotent cells or differentiated cells. In some embodiments, a population of mature stem cell-derived beta cells or precursors thereof is substantially free or devoid of embryonic stem cells or pluripotent cells or iPS cells.

Mature Stem Cell-Derived Beta Cells

In some embodiments, the disclosure provides mature stem cell-derived beta cells. The mature stem cell-derived beta cells disclosed herein share many distinguishing features of native mature beta cells, but are different in certain aspects (e.g., gene expression profiles). In some embodiments, the mature stem cell-derived beta cell is non-native or non-naturally occurring. As used herein, “non-native” or “non-naturally occurring” means that the mature beta cell (e.g., mature stem cell-derived beta cell) is markedly different in certain aspects from mature beta cells which exist in nature, i.e., native beta cells. In should be appreciated, however, that these marked differences typically pertain to structural features which may result in the mature stem cell-derived beta cells exhibiting certain functional differences, e.g., although the gene expression patterns of mature stem cell-derived beta cells differ from native mature beta cells, the mature stem cell-derived beta cells behave in a similar manner to native mature beta cells but certain functions may be altered (e.g., improved) compared to native mature beta cells.

The mature stem cell-derived beta cells of the disclosure share many characteristic features of native mature beta cells which are important for normal beta cell function. In some embodiments, the mature stem cell-derived beta cell exhibits a glucose stimulated insulin secretion (GSIS) response in vitro. In some embodiments, the mature stem cell-derived beta cell exhibits a GSIS response in vivo. In some embodiments, the mature stem cell-derived beta cell exhibits in vitro and in vivo GSIS responses. In some embodiments, the GSIS responses resemble the GSIS responses of an endogenous mature pancreatic β cell. In some embodiments, the mature stem cell-derived beta cell exhibits a GSIS response to at least one glucose challenge. In some embodiments, the mature stem cell-derived beta cell exhibits a GSIS response to at least two sequential glucose challenges. In some embodiments, the mature stem cell-derived beta cell exhibits a GSIS response to at least three sequential glucose challenges. In some embodiments, the GSIS responses resemble the GSIS response of endogenous human islets to multiple glucose challenges. In some embodiments, the GSIS response is observed immediately upon transplanting the cell into a human or animal. In some embodiments, the GSIS response is observed within approximately 24 hours of transplanting the cell into a human or animal. In some embodiments, the GSIS response is observed within approximately one week of transplanting the cell into a human or animal. In some embodiments, the GSIS response is observed within approximately two weeks of transplanting the cell into a human or animal. In some embodiments, the stimulation index of the cell as characterized by the ratio of insulin secreted in response to high glucose concentrations compared to low glucose concentrations is similar to the stimulation index of an endogenous mature pancreatic β cell. In some embodiments, the mature stem cell-derived beta cell exhibits a stimulation index of greater than 1. In some embodiments, the mature stem cell-derived beta cell exhibits a stimulation index of greater than or equal to 1. In some embodiments, the mature stem cell-derived beta cell exhibits a stimulation index of greater than 1.1. In some embodiments, the mature stem cell-derived beta cell exhibits a stimulation index of greater than or equal to 1.1. In some embodiments, the mature stem cell-derived beta cell exhibits a stimulation index of greater than 2. In some embodiments, the mature stem cell-derived beta cell exhibits a stimulation index of greater than or equal to 2. In some embodiments, the mature stem cell-derived beta cell exhibits a stimulation index of at least 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or greater.

In some embodiments, the mature stem cell-derived beta cell exhibits cytokine-induced apoptosis in response to cytokines. In some embodiments, the mature stem cell-derived beta cell exhibits cytokine-induced apoptosis in response to a cytokine selected from the group consisting of interleukin-1β (IL-β), interferon-γ (INF-γ), tumor necrosis factor-α (TNF-α), and combinations thereof.

In some embodiments, insulin secretion from the mature stem cell-derived beta cell is enhanced in response to known anti-diabetic drugs (e.g., anti-diabetic drugs which act on β cells ex vivo or in vitro, and/or anti-diabetic drugs generally in vivo). The disclosure contemplates any known anti-diabetic drug. In some embodiments, insulin secretion from the mature stem cell-derived beta cell is enhanced in response to a secretagogue. In some embodiments, the secretagogue is selected from the group consisting of an incretin mimetic, a sulfonylurea, a meglitinide, and combinations thereof.

In some embodiments, the mature stem cell-derived beta cell is monohormonal. In some embodiments, the mature stem cell-derived beta cell exhibits a morphology that resembles the morphology of an endogenous mature pancreatic β cell. In some embodiments, the mature stem cell-derived beta cell encapsulates crystalline insulin granules. In some embodiments, the mature stem cell-derived beta cell exhibits encapsulated crystalline insulin granules under electron microscopy that resemble insulin granules of an endogenous mature pancreatic β cell. In some embodiments, the mature stem cell-derived beta cell exhibits a low rate of replication. In some embodiments, the mature stem cell-derived beta cell exhibits a low rate of replication. In some embodiments, the mature stem cell-derived beta cell exhibits a low, but increased rate of replication as measured by staining for C-peptide and Ki67 in response to treatment with prolactin.

In some embodiments, the mature stem cell-derived beta cell increases intracellular Ca²⁺ in response to glucose. In some embodiments, the mature stem cell-derived beta cell exhibits a glucose stimulated Ca²⁺ flux (GSCF) that resembles the GSCF of an endogenous mature pancreatic β cell. In some embodiments, the mature stem cell-derived beta cell exhibits a GSCF response to at least three sequential glucose challenges in a manner that resembles the GSCF response of an endogenous mature pancreatic β cell to multiple glucose challenges.

In some embodiments, the mature stem cell-derived beta cell expresses at least one marker characteristic of an endogenous mature pancreatic β cell selected from the group consisting of insulin, C-peptide, PDX1, MAFA, NKX6-1, PAX6, NEUROD1, glucokinase (GCK), SLC2A1, PCSK1, KCNJ11, ABCC8, SLC30A8, SNAP25, RAB3A, GAD2, and PTPRN.

In some embodiments, the mature stem cell-derived beta cell does not express at least one marker (e.g., a marker not expressed by endogenous mature pancreatic β cells) selected from the group consisting of a) a hormone selected from the group consisting of i) glucagon (GCG), and ii) somatostatin (SST); b) an acinar cell marker selected from the group consisting of i) amylase, and ii) carboxypeptdase A (CPA1), c) an α cell marker selected from the group consisting of i) GCG, Arx, Irx1, and Irx2, d) a ductal cell marker selected from the group consisting of i) CFTR, and ii) Sox9.

The mature stem cell-derived beta cells are differentiated in vitro from any starting cell as the invention is not intended to be limited by the starting cell from which the mature stem cell-derived beta cells are derived. Exemplary starting cells include, without limitation, immature beta cells or any precursor thereof, such as an insulin-positive endocrine cell, a Nkx6-1-positive pancreatic progenitor cell, a Pdx1-positive pancreatic progenitor cell, a pluripotent stem cell, an embryonic stem cell, and an induced pluripotent stem cell. In some embodiments, the mature stem cell-derived beta cells are differentiated in vitro from a reprogrammed cell, a partially reprogrammed cell (i.e., a somatic cell, e.g., a fibroblast which has been partially reprogrammed such that it exists in an intermediate state between an induced pluripotency cell and the somatic cell from which it has been derived), a transdifferentiated cell. In some embodiments, the mature stem cell-derived beta cells disclosed herein can be differentiated in vitro from an immature beta cell (e.g., an immature stem cell-derived beta cell) or a precursor thereof. In some embodiments, the mature stem cell-derived beta cell is differentiated in vitro from a precursor selected from the group consisting of an insulin-positive endocrine cell, a Nkx6-1-positive pancreatic progenitor cell, a Pdx1-positive pancreatic progenitor cell, and a pluripotent stem cell. In some embodiments, the pluripotent stem cell is selected from the group consisting of an embryonic stem cell and induced pluripotent stem cell. In some embodiments, the mature stem cell-derived beta cell or the pluripotent stem cell from which the mature stem cell-derived beta cell is derived is human. In some embodiments, the mature stem cell-derived beta cell is human.

In some embodiments, the mature stem cell-derived beta cell is not genetically modified. In some embodiments, the mature stem cell-derived beta cell obtains the features it shares in common with native β cells in the absence of a genetic modification of cells. In some embodiments, the mature stem cell-derived beta cell is genetically modified.

In some embodiments, the insulin produced per mature stem cell-derived beta cell is at least 0.5 μIU per 1000 cells per 30 minute incubation (e.g., ex vivo) at a high glucose concentration.

In some embodiments, the insulin produced per mature stem cell-derived beta cell is at least 1, at least 2, at least 3, at least 4 at least 5 at least 6, at least 7 at least 8 or at least 9 μIU per 1000 cells per 30 minute incubation at a high glucose concentration. In some embodiments, the insulin produced per mature stem cell-derived beta cell is between 0.5 and 10 μIU per 1000 cells per 30 minute incubation at a high glucose concentration. In some embodiments, the insulin produced per mature stem cell-derived beta cell is approximately 2.5 μIU per 1000 cells per 30 minute incubation at a high glucose concentration.

In some aspects, the disclosure provides a cell line comprising a mature stem cell-derived beta cell described herein. In some embodiments, the mature stem cell-derived beta cells stably express insulin. In some embodiments, the mature stem cell-derived beta cell can be frozen, thawed, and amplified with a doubling time of 24 to 44 hours without significant morphological changes until at least 30 passages.

Aspects of the disclosure relate to isolated populations of mature stem cell-derived beta cells produced according to methods described herein. In some embodiments, a population of mature stem cell-derived beta cells is produced by contacting at least one immature beta cell with at least one maturation factor described herein.

Aspects of the disclosure involve microcapsules comprising isolated populations of cells described herein (e.g., mature stem cell-derived beta cells). Microcapsules are well known in the art. Suitable examples of microcapsules are described in the literature (e.g., Orive et al., “Application of cell encapsulation for controlled delivery of biological therapeutics”, Advanced Drug Delivery Reviews (2013), dx.doi.org/10.1016/j.addr.2013.07.009; Hernandez et al., “Microcapsules and microcarriers for in situ cell delivery”, Advanced Drug Delivery Reviews 2010; 62:711-730; Murua et al., “Cell microencapsulation technology: Towards clinical application”, Journal of Controlled Release 2008; 132:76-83; and Zanin et al., “The development of encapsulated cell technologies as therapies for neurological and sensory diseases”, Journal of Controlled Release 2012; 160:3-13). Microcapsules can be formulated in a variety of ways. Exemplary microcapsules comprise an alginate core surrounded by a polycation layer covered by an outer alginate membrane. The polycation membrane forms a semipermeable membrane, which imparts stability and biocompatibility. Examples of polycations include, without limitation, poly-L-lysine, poly-L-ornithine, chitosan, lactose modified chitosan, and photopolymerized biomaterials. In some embodiments, the alginate core is modified, for example, to produce a scaffold comprising an alginate core having covalently conjugated oligopeptides with an RGD sequence (arginine, glycine, aspartic acid). In some embodiments, the alginate core is modified, for example, to produce a covalently reinforced microcapsule having a chemoenzymatically engineered alginate of enhanced stability. In some embodiments, the alginate core is modified, for example, to produce membrane-mimetic films assembled by in-situ polymerization of acrylate functionalized phospholipids. In some embodiments, microcapsules are composed of enzymatically modified alginates using epimerases. In some embodiments, microcapsules comprise covalent links between adjacent layers of the microcapsule membrane. In some embodiment, the microcapsule comprises a subsieve-size capsule comprising alginate coupled with phenol moieties. In some embodiments, the microcapsule comprises a scaffold comprising alginate-agarose. In some embodiments, the mature stem cell-derived beta cell is modified with PEG before being encapsulated within alginate. In some embodiments, the isolated populations of cells, e.g., mature stem cell-derived beta cells are encapsulated in photoreactive liposomes and alginate. It should be appreciated that the alginate employed in the microcapsules can be replaced with other suitable biomaterials, including, without limitation, PEG, chitosan, PES hollow fibers, collagen, hyaluronic acid, dextran with RGD, EHD and PEGDA, PMBV and PVA, PGSAS, agarose, agarose with gelatin, PLGA, and multilayer embodiments of these.

In some embodiments, compositions comprising populations of mature stem cell-derived beta cells produced according to the methods described herein can also be used as the functional component in a mechanical device. For example, a device may contain a population of mature stem cell-derived beta cells (e.g., produced from populations of immature beta cells or precursors thereof) behind a semipermeable membrane that prevents passage of the cell population, retaining them in the device. Other examples of devices include those contemplated for either implantation into a diabetic patient, or for extracorporeal therapy.

Aspects of the disclosure involve assays comprising isolated populations of mature stem cell-derived beta cells described herein. In some embodiments, the assays can be used for identifying one or more candidate agents which promote or inhibit a mature stem cell-derived beta cell fate. In some embodiments, the assays can be used for identifying one or more candidate agents which promote the differentiation of at least one immature beta cell or a precursor thereof into mature stem cell-derived beta cells.

The disclosure contemplates methods in which mature stem cell-derived beta cells are generated according to the methods described herein from iPS cells derived from cells extracted or isolated from individuals suffering from a disease (e.g., diabetes, obesity, or a β cell-related disorder), and those mature stem cell-derived beta cells are compared to normal β cells from healthy individuals not having the disease to identify differences between the mature stem cell-derived beta cells and normal β cells which could be useful as markers for disease (e.g., epigenetic and/or genetic). In some embodiments, β cells are obtained from a diabetic individual and compared to normal β cells, and then the β cells are reprogrammed to iPS cells and the iPS cells are analyzed for genetic and/or epigenetic markers which are present in the β cells obtained from the diabetic individual but not present in the normal β cells, to identify markers (e.g., pre-diabetic). In some embodiments, the iPS cells and/or mature stem cell-derived cells derived from diabetic patients are used to screen for agents (e.g., agents which are able to modulate genes contributing to a diabetic phenotype).

Confirmation of the Presence and the Identification of Mature Stem Cell-Derived Beta Cells

One can use any means common to one of ordinary skill in the art to confirm the presence of a mature stem cell-derived beta cell produced by the differentiation of at least one immature beta cell or precursor thereof by exposure to at least one maturation factor as described herein.

In some embodiments, the presence of mature beta cell markers, e.g. chemically induced beta cells, can be done by detecting the presence or absence of one or more markers indicative of an endogenous mature beta cell. In some embodiments, the method can include detecting the positive expression (e.g. the presence) of a marker for mature beta cells. In some embodiments, the marker can be detected using a reagent, e.g., a reagent for the detection of NKX6-1 and C-peptide. In particular, mature stem cell-derived beta cells herein express NKX6-1 and C-peptide, and do not express significant levels of other markers which would be indicative of immature beta cells (e.g., MafB).

A reagent for a marker can be, for example, an antibody against the marker or primers for a RT-PCR or PCR reaction, e.g., a semi-quantitative or quantitative RT-PCR or PCR reaction. Such markers can be used to evaluate whether a mature stem cell-derived beta cell has been produced. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The progression of at least one immature beta cell or precursor thereof to a mature stem cell-derived beta cell can be monitored by determining the expression of markers characteristic of mature beta cells. In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of mature beta cells as well as the lack of significant expression of markers characteristic of immature beta cells or precursors thereof from which it was derived is determined.

As described in connection with monitoring the production of a mature stem cell-derived beta cell from an immature beta cell, qualitative or semi-quantitative techniques, such as blot transfer methods and immunocytochemistry, can be used to measure marker expression, using methods commonly known to persons of ordinary skill in the art. Alternatively, marker expression can be accurately quantitated through the use of technique such as quantitative-PCR by methods ordinarily known in the art. Additionally, techniques for measuring extracellular marker content, such as ELISA, may be utilized.

Mature stem cell-derived beta cells can also be characterized by the down-regulation of markers characteristic of the pluripotent stem from which the mature stem cell-derived beta cell is induced from. For example, mature stem cell-derived beta cells derived from pluripotent stem cells may be characterized by a statistically significant down-regulation of the pluripotent stem cell markers alkaline phosphatase (AP), NANOG, OCT-4, SOX-2, SSEA4, TRA-1-60 or TRA-1-81 in the mature beta cell relative to the expression in the pluripotent stem cell from which it was derived. Other markers expressed by pluripotent cell markers, include but are not limited to alkaline phosphatase (AP); ABCG2; stage specific embryonic antigen-1 (SSEA-1); SSEA-3; SSEA-4; TRA-1-60; TRA-1-81; Tra-2-49/6E; ERas/ECATS, E-cadherin; βIII-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPAS/ECAT2; ECAT3; ECAT6; ECAT7; ECAT8; ECAT9; ECAT10; ECAT15-1; ECAT15-2; Fth117; Sa114; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; Dnmt3L; Sox15; Stat3; Grb2; SV40 Large T Antigen; HPV16 E6; HPV16 E7, β-catenin, and Bmi1 and other general markers for pluripotency, etc, and at least one or more of these are down regulated by a statistically significant amount in a mature beta cell as compared to the pluripotent stem cell from which they were derived.

It is understood that the present invention is not limited to those markers listed as mature beta cell markers herein, and the present invention also encompasses markers such as cell surface markers, antigens, and other gene products including ESTs, RNA (including microRNAs and antisense RNA), DNA (including genes and cDNAs), and portions thereof.

Enrichment, Isolation and Purification of Mature Stem Cell-Derived Beta Cells

Another aspect of the present invention relates to the isolation of a population of mature stem cell-derived beta cells from a heterogeneous population of cells, such as a mixed population of cells comprising mature stem cell-derived beta cells and immature beta cells (e.g., immature stem cell-derived beta cells) or precursors thereof from which the mature stem cell-derived beta cells was derived. A population of mature stem cell-derived beta cells produced by any of the above-described processes can be enriched, isolated and/or purified by using any cell surface marker present on the mature stem cell-derived beta cells which is not present on the immature beta cells or precursor thereof from which it was derived. Such cell surface markers are also referred to as an affinity tag which is specific for a mature stem cell-derived beta cell. Examples of affinity tags specific for mature stem cell-derived beta cells are antibodies, ligands or other binding agents that are specific to a marker molecule, such as a polypeptide, that is present on the cell surface of a mature stem cell-derived beta cell but which is not substantially present on other cell types (e.g. immature beta cells or precursors thereof). In some processes, an antibody which binds to a cell surface antigen on a mature stem cell-derived beta cell is used as an affinity tag for the enrichment, isolation or purification of chemically induced (e.g. by contacting with at least one maturation factor as described herein) mature stem cell-derived beta cell produced by the methods described herein. Such antibodies are known and commercially available.

The skilled artisan will readily appreciate the processes for using antibodies for the enrichment, isolation and/or purification of mature stem cell-derived beta cells. For example, in some embodiments, the reagent, such as an antibody, is incubated with a cell population comprising mature stem cell-derived beta cells, wherein the cell population has been treated to reduce intercellular and substrate adhesion. The cell population is then washed, centrifuged and resuspended. In some embodiments, if the antibody is not already labeled with a label, the cell suspension is then incubated with a secondary antibody, such as an FITC-conjugated antibody that is capable of binding to the primary antibody. The mature stem cell-derived beta cells are then washed, centrifuged and resuspended in buffer. The mature stem cell-derived beta cell suspension is then analyzed and sorted using a fluorescence activated cell sorter (FACS). Antibody-bound, fluorescent reprogrammed cells are collected separately from non-bound, non-fluorescent cells, thereby resulting in the isolation of mature stem cell-derived beta cells from other cells present in the cell suspension, e.g. immature beta cells or precursors thereof.

In another embodiment of the processes described herein, the isolated cell composition comprising mature stem cell-derived beta cells can be further purified by using an alternate affinity-based method or by additional rounds of sorting using the same or different markers that are specific for mature stem cell-derived beta cells. For example, in some embodiments, FACS sorting is used to first isolate a mature stem cell-derived beta cell which expresses NKX6-1, either alone or with the expression of C-peptide, or alternatively with a β cell marker disclosed herein from cells that do not express one of those markers (e.g. negative cells) in the cell population. A second FAC sorting, e.g. sorting the positive cells again using FACS to isolate cells that are positive for a different marker than the first sort enriches the cell population for reprogrammed cells.

In an alternative embodiment, FACS sorting is used to separate cells by negatively sorting for a marker that is present on most immature beta cells or precursors thereof, but is not present on mature stem cell-derived beta cell.

In some embodiments of the processes described herein, mature stem cell-derived beta cells are fluorescently labeled without the use of an antibody then isolated from non-labeled cells by using a fluorescence activated cell sorter (FACS). In such embodiments, a nucleic acid encoding GFP, YFP or another nucleic acid encoding an expressible fluorescent marker gene, such as the gene encoding luciferase, is used to label reprogrammed cells using the methods described above.

In addition to the procedures just described, chemically induced mature stem cell-derived beta cells may also be isolated by other techniques for cell isolation. Additionally, mature stem cell-derived beta cells may also be enriched or isolated by methods of serial subculture in growth conditions which promote the selective survival or selective expansion of the mature stem cell-derived beta cells. Such methods are known by persons of ordinary skill in the art, and may include the use of agents such as, for example, insulin, members of the TGF-beta family, including Activin A, TGF-beta1, 2, and 3, bone morphogenic proteins (BMP-2, -3, -4, -5, -6, -7, -11, -12, and -13), fibroblast growth factors-1 and -2, platelet-derived growth factor-AA, and -BB, platelet rich plasma, insulin-like growth factors (IGF-I, II) growth differentiation factor (GDF-5, -6, -7, -8, -10, -11, -15), vascular endothelial cell-derived growth factor (VEGF), Hepatocyte growth factor (HGF), pleiotrophin, endothelin, Epidermal growth factor (EGF), beta-cellulin, among others. Other pharmaceutical compounds can include, for example, nicotinamide, glucagon like peptide-I (GLP-1) and II, GLP-1 and 2 mimetibody, Exendin-4, retinoic acid, parathyroid hormone.

Using the methods described herein, enriched, isolated and/or purified populations of mature stem cell-derived beta cells can be produced in vitro from immature beta cells or precursors thereof (which were differentiated from pluripotent stem cells by the methods described herein). In some embodiments, preferred enrichment, isolation and/or purification methods relate to the in vitro production of human mature stem cell-derived beta cells from human immature beta cells or precursors thereof, which were differentiated from human pluripotent stem cells, or from human induced pluripotent stem (iPS) cells. In such an embodiment, where mature stem cell-derived beta cells are differentiated or matured from immature beta cells, which were previously derived from iPS cells, the mature stem cell-derived beta cells can be autologous to the subject from whom the cells were obtained to generate the iPS cells.

Using the methods described herein, isolated cell populations of mature stem cell-derived beta cells are enriched in mature stem cell-derived beta cell content by at least about 1- to about 1000-fold as compared to a population of cells before the chemical induction of the immature beta cells or precursor population. In some embodiments the population of mature stem cell-derived beta cells is induced, enhanced, enriched, or increased by at least 5%, 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more as compared to a population of cells before the chemical induction of immature beta cells or precursor population.

Compositions Comprising Mature Stem Cell-Derived Beta Cells

Some embodiments of the present invention relate to cell compositions, such as cell cultures or cell populations, comprising mature stem cell-derived beta cells, wherein the mature stem cell-derived beta cells have been derived from at least one immature beta cell (e.g., an immature stem cell-derived beta cell). In some embodiments, the cell compositions comprise immature beta cells.

In accordance with certain embodiments, the chemically induced mature stem cell-derived beta cells are mammalian cells, and in a preferred embodiment, such mature stem cell-derived beta cells are human mature stem cell-derived beta cells. In some embodiments, the immature beta cells have been derived from pluripotent stem cells (e.g., human pluripotent stem cells).

Other embodiments of the present invention relate to compositions, such as an isolated cell population or cell culture, comprising mature stem cell-derived beta cells produced by the methods as disclosed herein. In such embodiments, the mature stem cell-derived beta cells comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the mature stem cell-derived beta cell population. In some embodiments, the composition comprises a population of mature stem cell-derived beta cells which make up more than about 90% of the total cells in the cell population, for example about at least 95%, or at least 96%, or at least 97%, or at least 98% or at least about 99%, or about at least 100% of the total cells in the cell population are mature stem cell-derived beta cells.

Certain other embodiments of the present invention relate to compositions, such as an isolated cell population or cell cultures, comprising a combination of mature stem cell-derived beta cells and immature beta cells or precursors thereof from which the mature stem cell-derived beta cells were derived. In some embodiments, the immature beta cells from which the mature stem cell-derived beta cells are derived comprise less than about 25%, less than about 20%, less than about 15%, less than about 10%, less than about 5%, less than about 4%, less than about 3%, less than about 2% or less than about 1% of the total cells in the isolated cell population or culture.

Additional embodiments of the present invention relate to compositions, such as isolated cell populations or cell cultures, produced by the processes described herein and which comprise chemically induced mature stem cell-derived beta cells as the majority cell type. In some embodiments, the methods and processes described herein produce an isolated cell culture and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% mature stem cell-derived beta cells.

In another embodiment, isolated cell populations or compositions of cells (or cell cultures) comprise human mature stem cell-derived beta cells. In other embodiments, the methods and processes as described herein can produce isolated cell populations comprising at least about 50%, at least about 45%, at least about 40%, at least about 35%, at least about 30%, at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2% or at least about 1% mature stem cell-derived beta cells. In preferred embodiments, isolated cell populations can comprise human mature stem cell-derived beta cells. In some embodiments, the percentage of mature stem cell-derived beta cells in the cell cultures or populations is calculated without regard to the feeder cells remaining in the culture.

Still other embodiments of the present invention relate to compositions, such as isolated cell populations or cell cultures, comprising mixtures of mature stem cell-derived beta cells and immature beta cells or precursors thereof from which they were differentiated or matured from. For example, cell cultures or cell populations comprising at least about 5 mature stem cell-derived beta cells for about every 95 immature beta cells or precursors thereof can be produced. In other embodiments, cell cultures or cell populations comprising at least about 95 mature stem cell-derived beta cells for about every 5 immature beta cells or precursors thereof can be produced. Additionally, cell cultures or cell populations comprising other ratios of mature stem cell-derived beta cells to immature beta cells or precursors thereof are contemplated. For example, compositions comprising at least about 1 mature stem cell-derived beta cell for about every 1,000,000, or at least 100,000 cells, or at least 10,000 cells, or at least 1000 cells or 500, or at least 250 or at least 100 or at least 10 immature beta cells or precursors thereof can be produced.

Further embodiments of the present invention relate to compositions, such as cell cultures or cell populations, comprising human cells, including human mature stem cell-derived beta cells, which displays at least one characteristic of an endogenous mature beta cell.

In preferred embodiments of the present invention, cell cultures and/or cell populations of mature stem cell-derived beta cells comprise human mature stem cell-derived beta cells that are non-recombinant cells. In such embodiments, the cell cultures and/or cell populations are devoid of or substantially free of recombinant human mature stem cell-derived beta cells.

Maturation Factors

Aspects of the disclosure involve contacting immature beta cells or precursors thereof with one or more maturation factors, for example, to induce the maturation of the immature beta cells or differentiation of the precursors thereof into stem cell-derived beta cells (e.g., mature stem cell-derived beta cells). The term “maturation factor” refers to an agent that promotes or contributes to the conversion of at least one immature beta cell or a precursor thereof to a stem cell-derived beta cell. In some embodiments, the maturation factor induces the differentiation of pluripotent cells (e.g., iPSCs or hESCs) into immature beta cells, e.g., in accordance with a method described herein. In some embodiments, the maturation factor induces the maturation of immature beta cells into mature stem cell-derived beta cells, e.g., in accordance with a method described herein.

Generally, at least one maturation factor described herein can be used alone, or in combination with other maturation factors, to generate stem cell-derived beta cells according to the methods as disclosed herein. In some embodiments, at least two, at least three, at least four, at least five, at least six, at least seven, at least eight, at least nine, or at least ten maturation factors described herein are used in the methods of generating stem cell-derived beta cells (e.g., mature stem cell-derived beta cells).

In some embodiments, a maturation factor comprises a modulator (e.g., inhibitor) of the phosphoinositide 3-kinase (PI3K)/Akt/mTOR pathway. In some embodiments, a maturation factor comprises an inhibitor of the mTOR pathway. In some embodiments, a maturation factor comprises an inhibitor of PI3K and/or Akt. In some embodiments, a maturation factor comprises a small molecule, nucleic acid, amino acid, metabolite, polypeptide, antibody and antibody-like molecules, aptamers, macrocycles, or other molecules. In some embodiments, a maturation factor is selected from the group consisting of Torin1, Torin2, rapamycin, everolimus and temsirolimus. In some embodiments, a maturation factor is Torin1. In some embodiments, a maturation factor is Torin2. In some embodiments, a maturation factor is rapamycin. In some embodiments, a maturation factor is everolimus. In some embodiments, a maturation factor is temsirolimus.

### Compositions and Kits

Described herein are compositions which comprise a cell described herein (e.g., a mature stem cell-derived beta cell or mature pancreatic beta cell). In some embodiments, the composition also includes a maturation factor described herein and/or cell culture media. Described herein are also compositions comprising the compounds described herein (e.g., cell culture media comprising one or more of the compounds described herein).

Also described herein are kits for practicing methods disclosed herein and for making mature stem cell-derived beta cells or mature pancreatic beta cells disclosed herein. In one aspect, a kit includes at least one immature beta cell or precursor thereof and at least one maturation factor as described herein, and optionally, the kit can further comprise instructions for converting at least one immature beta cell or precursor thereof to a population of mature stem cell-derived beta cells using a method described herein. In some embodiments, the kit comprises at least two maturation factors. In some embodiments, the kit comprises at least three maturation factors. In some embodiments, the kit comprises at least four maturation factors.

In some embodiment, the compound in the kit can be provided in a watertight or gas tight container which in some embodiments is substantially free of other components of the kit. The compound can be supplied in more than one container, e.g., it can be supplied in a container having sufficient reagent for a predetermined number of reactions e.g., 1, 2, 3 or greater number of separate reactions to induce immature beta cells, or precursors thereof, into mature stem cell-derived beta cells. A maturation factor can be provided in any form, e.g., liquid, dried or lyophilized form. It is preferred that a compound(s) (e.g., maturation factor) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

In some embodiments, the kit further optionally comprises information material. The informational material can be descriptive, instructional, marketing or other material that relates to the methods described herein and/or the use of a compound(s) described herein for the methods described herein.

The informational material of the kits is not limited in its instruction or informative material. In one embodiment, the informational material can include information about production of the compound, molecular weight of the compound, concentration, date of expiration, batch or production site information, and so forth. In one embodiment, the informational material relates to methods for administering the compound. Additionally, the informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In one embodiment, the informational material can include instructions to administer a compound(s) (e.g., a maturation factor) as described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

The kit can include one or more containers for the composition containing at least one maturation factor as described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The kit can also include a component for the detection of a marker for mature stem cell-derived beta cells, e.g., for a marker described herein, e.g., a reagent for the detection of mature stem cell-derived beta cells. Or in some embodiments, the kit can also comprise reagents for the detection of negative markers of mature stem cell-derived beta cells for the purposes of negative selection of mature stem cell-derived beta cells or for identification of cells which do not express these negative markers (e.g., mature stem cell-derived beta cells). The reagents can be, for example, an antibody against the marker or primers for a RT-PCR or PCR reaction, e.g., a semi-quantitative or quantitative RT-PCR or PCR reaction. Such markers can be used to evaluate whether an iPS cell has been produced. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit can include stem cell-derived beta cells, e.g., mature stem cell-derived beta cells derived from the same type of immature beta cells or precursor thereof, for example for the use as a positive cell type control.

Methods of Administering a Cell

In one embodiment, the cells described herein, e.g. a population of mature stem cell-derived beta cells is transplantable, e.g., a population of mature stem cell-derived beta cells can be administered to a subject. In some embodiments, the subject who is administered a population of mature stem cell-derived beta cells is the same subject from whom a pluripotent stem cell used to differentiate into a mature stem cell-derived beta cell was obtained (e.g. for autologous cell therapy). In some embodiments, the subject is a different subject. In some embodiments, a subject is suffering from diabetes such as type I diabetes, or is a normal subject. For example, the cells for transplantation (e.g. a composition comprising a population of mature stem cell-derived beta cells) can be a form suitable for transplantation, e.g., organ transplantation.

The method can further include administering the cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

A composition comprising a population of mature stem cell-derived beta cells can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Pharmaceutical Compositions

For administration to a subject, a cell population produced by the methods as disclosed herein, e.g. a population of mature stem cell-derived beta cells (produced by contacting at least one immature beta cell with at least one maturation factor (e.g., any one, two, three, or more maturation factors as described herein) can be administered to a subject, for example in pharmaceutically acceptable compositions. These pharmaceutically acceptable compositions comprise a therapeutically-effective amount of a population of mature stem cell-derived beta cells as described above, formulated together with one or more pharmaceutically acceptable carriers (additives) and/or diluents.

As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. Nos. 3,773,919; and 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein in respect to a population of cells means that amount of relevant cells in a population of cells, e.g., mature stem cell-derived beta cells, or composition comprising mature stem cell-derived beta cells of the present invention which is effective for producing some desired therapeutic effect in at least a sub-population of cells in an animal at a reasonable benefit/risk ratio applicable to any medical treatment. For example, an amount of a population of mature stem cell-derived beta cells administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

By “treatment”, “prevention” or “amelioration” of a disease or disorder is meant delaying or preventing the onset of such a disease or disorder, reversing, alleviating, ameliorating, inhibiting, slowing down or stopping the progression or severity of a condition associated with such a disease or disorder. In one embodiment, the symptoms of a disease or disorder are alleviated by at least 5%, at least 10%, at least 20%, at least 30%, at least 40%, or at least 50%.

As used herein, the term “administer” refers to the placement of a composition into a subject by a method or route which results in at least partial localization of the composition at a desired site such that the desired effect is produced. Routes of administration suitable for the methods of the invention include both local and systemic administration. Generally, local administration results in more of the administered stem cell-derived beta cells being delivered to a specific location as compared to the entire body of the subject, whereas, systemic administration results in delivery of the stem cell-derived beta cells to essentially the entire body of the subject.

In the context of administering a compound treated cell, the term “administering” also include transplantation of such a cell in a subject. As used herein, the term “transplantation” refers to the process of implanting or transferring at least one cell to a subject. The term “transplantation” includes, e.g., autotransplantation (removal and transfer of cell(s) from one location on a patient to the same or another location on the same patient), allotransplantation (transplantation between members of the same species), and xenotransplantation (transplantation between members of different species).

Mature stem cell-derived beta cells or compositions comprising the same can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

Treatment of Diabetes is determined by standard medical methods. A goal of Diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patent, depending on other factors, such as how often the patient has low blood sugar reactions. Useful medical tests include tests on the patient's blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to Diabetes, such as diseases of the eye, kidney disease, or nerve disease.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female. A subject can be one who has been previously diagnosed with or identified as suffering from or having Diabetes (e.g., Type 1 or Type 2), one or more complications related to Diabetes, or a pre-diabetic condition, and optionally, but need not have already undergone treatment for the Diabetes, the one or more complications related to Diabetes, or the pre-diabetic condition. A subject can also be one who is not suffering from Diabetes or a pre-diabetic condition. A subject can also be one who has been diagnosed with or identified as suffering from Diabetes, one or more complications related to Diabetes, or a pre-diabetic condition, but who show improvements in known Diabetes risk factors as a result of receiving one or more treatments for Diabetes, one or more complications related to Diabetes, or the pre-diabetic condition. Alternatively, a subject can also be one who has not been previously diagnosed as having Diabetes, one or more complications related to Diabetes, or a pre-diabetic condition. For example, a subject can be one who exhibits one or more risk factors for Diabetes, complications related to Diabetes, or a pre-diabetic condition, or a subject who does not exhibit Diabetes risk factors, or a subject who is asymptomatic for Diabetes, one or more Diabetes-related complications, or a pre-diabetic condition. A subject can also be one who is suffering from or at risk of developing Diabetes or a pre-diabetic condition. A subject can also be one who has been diagnosed with or identified as having one or more complications related to Diabetes or a pre-diabetic condition as defined herein, or alternatively, a subject can be one who has not been previously diagnosed with or identified as having one or more complications related to Diabetes or a pre-diabetic condition.

As used herein, the phrase “subject in need of stem cell-derived beta cells” refers to a subject who is diagnosed with or identified as suffering from, having or at risk for developing diabetes (e.g., Type 1, Type 1.5 or Type 2), one or more complications related to diabetes, or a pre-diabetic condition.

A subject in need of a population of mature stem cell-derived beta cells can be identified using any method used for diagnosis of diabetes. For example, Type 1 diabetes can be diagnosed using a glycosylated hemoglobin (A1C) test, a random blood glucose test and/or a fasting blood glucose test. Parameters for diagnosis of diabetes are known in the art and available to skilled artisan without much effort.

In some embodiments, the methods of the invention further comprise selecting a subject identified as being in need of additional mature stem cell-derived beta cells. A subject in need a population of mature stem cell-derived beta cells can be selected based on the symptoms presented, such as symptoms of type 1, type 1.5 or type 2 diabetes. Exemplary symptoms of diabetes include, but are not limited to, excessive thirst (polydipsia), frequent urination (polyuria), extreme hunger (polyphagia), extreme fatigue, weight loss, hyperglycemia, low levels of insulin, high blood sugar (e.g., sugar levels over 250 mg, over 300 mg), presence of ketones in urine, fatigue, dry and/or itchy skin, blurred vision, slow healing cuts or sores, more infections than usual, numbness and tingling in feet, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, and combinations thereof.

In some embodiments, a composition comprising a population of mature stem cell-derived beta cells for administration to a subject can further comprise a pharmaceutically active agent, such as those agents known in the art for treatment of diabetes and or for having anti-hyperglycemic activities, for example, inhibitors of dipeptidyl peptidase 4 (DPP-4) (e.g., Alogliptin, Linagliptin, Saxagliptin, Sitagliptin, Vildagliptin, and Berberine), biguanides (e.g., Metformin, Buformin and Phenformin), peroxisome proliferator-activated receptor (PPAR) modulators such as thiazolidinediones (TZDs) (e.g., Pioglitazone, Rivoglitazone, Rosiglitazone and Troglitazone), dual PPAR agonists (e.g., Aleglitazar, Muraglitazar and Tesaglitazar), sulfonylureas (e.g., Acetohexamide, Carbutamide, Chlorpropamide, Gliclazide, Tolbutamide, Tolazamide, Glibenclamide (Glyburide), Glipizide, Gliquidone, Glyclopyramide, and Glimepiride), meglitinides (“glinides”) (e.g., Nateglinide, Repaglinide and Mitiglinide), glucagon-like peptide-1 (GLP-1) and analogs (e.g., Exendin-4, Exenatide, Liraglutide, Albiglutide), insulin and insulin analogs (e.g., Insulin lispro, Insulin aspart, Insluin glulisine, Insulin glargine, Insulin detemir, Exubera and NPH insulin), alpha-glucosidase inhibitors (e.g., Acarbose, Miglitol and Voglibose), amylin analogs (e.g. Pramlintide), Sodium-dependent glucose cotransporter T2 (SGLT T2) inhibitors (e.g., Dapgliflozin, Remogliflozin and Sergliflozin) and others (e.g. Benfluorex and Tolrestat).

In type 1 diabetes, beta cells are undesirably destroyed by continued autoimmune response. Thus, this autoimmune response can be attenuated by use of compounds that inhibit or block such an autoimmune response. In some embodiments, a composition comprising a population of mature stem cell-derived beta cells for administration to a subject can further comprise a pharmaceutically active agent which is an immune response modulator. As used herein, the term “immune response modulator” refers to a compound (e.g., a small-molecule, antibody, peptide, nucleic acid, or gene therapy reagent) that inhibits autoimmune response in a subject. Without wishing to be bound by theory, an immune response modulator inhibits the autoimmune response by inhibiting the activity, activation, or expression of inflammatory cytokines (e.g., IL-12, IL-23 or IL-27), or STAT-4. Exemplary immune response modulators include, but are not limited to, members of the group consisting of Lisofylline (LSF) and the LSF analogs and derivatives described in U.S. Pat. No. 6,774,130, contents of which are herein incorporated by reference in their entirety.

A composition comprising mature stem cell-derived beta cells can be administered to the subject at the same time, or at different times as the administration of a pharmaceutically active agent or composition comprising the same. When administrated at different times, the compositions comprising a population of mature stem cell-derived beta cells and/or pharmaceutically active agent for administration to a subject can be administered within 5 minutes, 10 minutes, 20 minutes, 60 minutes, 2 hours, 3 hours, 4, hours, 8 hours, 12 hours, 24 hours of administration of the other. When a composition comprising a population of mature stem cell-derived beta cells and a composition comprising a pharmaceutically active agent are administered in different pharmaceutical compositions, routes of administration can be different. In some embodiments, a subject is administered a composition comprising mature stem cell-derived beta cells. In other embodiments, a subject is administered a composition comprising a pharmaceutically active agent. In another embodiment, a subject is administered a composition comprising a population of mature stem cell-derived beta cells mixed with a pharmaceutically active agent. In another embodiment, a subject is administered a composition comprising a population of mature stem cell-derived beta cells and a composition comprising a pharmaceutically active agent, where administration is substantially at the same time, or subsequent to each other.

Toxicity and therapeutic efficacy of administration of a compositions comprising a population of mature stem cell-derived beta cells can be determined by standard pharmaceutical procedures in cell cultures or experimental animals, e.g., for determining the LD50 (the dose lethal to 50% of the population) and the ED50 (the dose therapeutically effective in 50% of the population). Compositions comprising a population of mature stem cell-derived beta cells that exhibit large therapeutic indices, are preferred.

The amount of a composition comprising a population of mature stem cell-derived beta cells can be tested using several well-established animal models. Examples of such models are described in WO 2015/002724 and WO 2014/201167, which are incorporated herein by reference.

In some embodiments, data obtained from the cell culture assays and in animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. The therapeutically effective dose of a composition comprising a population of mature stem cell-derived beta cells can also be estimated initially from cell culture assays. A dose may be formulated in animal models in vivo to achieve a secretion of insulin at a concentration which is appropriate in response to circulating glucose in the plasma. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alterations to the treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules include administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of mature stem cell-derived beta cells as disclosed herein. In one embodiment of the invention, an isolated population of mature stem cell-derived beta cells as disclosed herein may be used for the production of a pharmaceutical composition, for the use in transplantation into subjects in need of treatment, e.g. a subject that has, or is at risk of developing diabetes, for example, but not limited to, subjects with congenital and acquired diabetes. In one embodiment, an isolated population of mature stem cell-derived beta cells may be genetically modified. In another aspect, the subject may have or be at risk of diabetes and/or metabolic disorder. In some embodiments, an isolated population of mature stem cell-derived beta cells as disclosed herein may be autologous and/or allogeneic. In some embodiments, the subject is a mammal, and in other embodiments the mammal is a human.

The use of an isolated population of mature stem cell-derived beta cells as disclosed herein provides advantages over existing methods because the population of mature stem cell-derived beta cells can be matured from immature beta cells or precursors thereof derived from stem cells, e.g. iPS cells obtained or harvested from the subject administered an isolated population of mature stem cell-derived beta cells. This is highly advantageous as it provides a renewable source of mature stem cell-derived beta cells which can be differentiated from stem cells to immature beta cells by methods commonly known by one of ordinary skill in the art, and then further differentiated by the methods described herein to pancreatic beta-like cells or cells with pancreatic beta cell characteristics, for transplantation into a subject, in particular a substantially pure population of mature pancreatic beta-like cells that do not have the risks and limitations of cells derived from other systems.

In another embodiment, an isolated population of mature stem cell-derived beta cells (e.g., mature pancreatic beta cells or beta-like cells) can be used as models for studying properties for the differentiation into insulin-producing cells, e.g. to pancreatic beta cells or pancreatic beta-like cells, or pathways of development of cells of endoderm origin into pancreatic beta cells.

In some embodiments, the immature beta cells or mature stem cell-derived beta cells may be genetically engineered to comprise markers operatively linked to promoters that are expressed when a marker is expressed or secreted, for example, a marker can be operatively linked to an insulin promoter, so that the marker is expressed when the immature beta cells or precursors thereof mature or differentiate into mature stem cell-derived beta cells which express and secrete insulin. In some embodiments, a population of mature stem cell-derived beta cells can be used as a model for studying the differentiation pathway of cells which differentiate into islet beta cells or pancreatic beta-like cells.

In other embodiments, the insulin-producing, glucose responsive cells can be used as models for studying the role of islet beta cells in the pancreas and in the development of diabetes and metabolic disorders. In some embodiments, the mature stem cell-derived beta cells can be from a normal subject, or from a subject which carries a mutation and/or polymorphism (e.g. in the gene Pdx1 which leads to early-onset insulin-dependent diabetes mellitus (NIDDM)), as well as maturity onset diabetes of the young type 4 (MODY4), which can be used to identify small molecules and other therapeutic agents that can be used to treat subjects with diabetes with a mutation or polymorphism in Pdx1. In some embodiments, the mature stem cell-derived beta cells may be genetically engineered to correct the polymorphism in the Pdx1 gene prior to being administered to a subject in the therapeutic treatment of a subject with diabetes. In some embodiments, the mature stem cell-derived beta cells may be genetically engineered to carry a mutation and/or polymorphism.

One embodiment of the invention relates to a method of treating diabetes or a metabolic disorder in a subject comprising administering an effective amount of a composition comprising a population of mature stem cell-derived beta cells as disclosed herein to a subject with diabetes and/or a metabolic disorder. In a further embodiment, the invention provides a method for treating diabetes, comprising administering a composition comprising a population of mature stem cell-derived beta cells as disclosed herein to a subject that has, or has increased risk of developing diabetes in an effective amount sufficient to produce insulin in response to increased blood glucose levels.

In one embodiment of the above methods, the subject is a human and a population of mature stem cell-derived beta cells as disclosed herein is human cells.

In some embodiments, the invention contemplates that a population of mature stem cell-derived beta cells as disclosed herein is administered directly to the pancreas of a subject, or is administered systemically. In some embodiments, a population of mature stem cell-derived beta cells as disclosed herein can be administered to any suitable location in the subject, for example in a capsule in the blood vessel or the liver or any suitable site where administered the population of mature stem cell-derived beta cells can secrete insulin in response to increased glucose levels in the subject.

The present invention is also directed to a method of treating a subject with diabetes or a metabolic disorder which occurs as a consequence of genetic defect, physical injury, environmental insult or conditioning, bad health, obesity and other diabetes risk factors commonly known by a person of ordinary skill in the art. Efficacy of treatment of a subject administered a composition comprising a population of mature stem cell-derived beta cells can be monitored by clinically accepted criteria and tests, which include for example, (i) Glycated hemoglobin (A1C) test, which indicates a subjects average blood sugar level for the past two to three months, by measuring the percentage of blood sugar attached to hemoglobin, the oxygen-carrying protein in red blood cells. The higher the blood sugar levels, the more hemoglobin has sugar attached. An A1C level of 6.5 percent or higher on two separate tests indicates the subject has diabetes. A test value of 6-6.5% suggest the subject has prediabetes. (ii) Random blood sugar test. A blood sample will be taken from the subject at a random time, and a random blood sugar level of 200 milligrams per deciliter (mg/dL)-11.1 millimoles per liter (mmol/L), or higher indicated the subject has diabetes. (iii) Fasting blood sugar test. A blood sample is taken from the subject after an overnight fast. A fasting blood sugar level between 70 and 99 mg/dL (3.9 and 5.5 mmol/L) is normal. If the subjects fasting blood sugar levels is 126 mg/dL (7 mmol/L) or higher on two separate tests, the subject has diabetes. A blood sugar level from 100 to 125 mg/dL (5.6 to 6.9 mmol/L) indicates the subject has prediabetes. (iv) Oral glucose tolerance test. A blood sample will be taken after the subject has fasted for at least eight hours or overnight and then ingested a sugary solution, and the blood sugar level will be measured two hours later. A blood sugar level less than 140 mg/dL (7.8 mmol/L) is normal. A blood sugar level from 140 to 199 mg/dL (7.8 to 11 mmol/L) is considered prediabetes. This is sometimes referred to as impaired glucose tolerance (IGT). A blood sugar level of 200 mg/dL (11.1 mmol/L) or higher may indicate diabetes.

In some embodiments, the effects of administration of a population of mature stem cell-derived beta cells as disclosed herein to a subject in need thereof is associated with improved exercise tolerance or other quality of life measures, and decreased mortality. The effects of cellular therapy with a population of mature stem cell-derived beta cells can be evident over the course of days to weeks after the procedure. However, beneficial effects may be observed as early as several hours after the procedure, and may persist for several years. In some embodiments, the effects of cellular therapy with a population of mature stem cell-derived beta cells occur within two weeks after the procedure.

In some embodiments, a population of mature stem cell-derived beta cells as disclosed herein may be used for tissue reconstitution or regeneration in a human patient or other subject in need of such treatment. In some embodiments compositions of populations of mature stem cell-derived beta cells can be administered in a manner that permits them to graft or migrate to the intended tissue site and reconstitute or regenerate the functionally deficient area. Special devices are available that are adapted for administering cells capable of reconstituting a population of beta cells in the pancreas or at an alternative desired location. Accordingly, the mature stem cell-derived beta cells may be administered to a recipient subject's pancreas by injection, or administered by intramuscular injection.

In some embodiments, compositions comprising a population of mature stem cell-derived beta cells as disclosed herein have a variety of uses in clinical therapy, research, development, and commercial purposes. For therapeutic purposes, for example, a population of mature stem cell-derived beta cells as disclosed herein may be administered to enhance insulin production in response to increase in blood glucose level for any perceived need, such as an inborn error in metabolic function, the effect of a disease condition (e.g. diabetes), or the result of significant trauma (i.e. damage to the pancreas or loss or damage to islet β cells). In some embodiments, a population of mature stem cell-derived beta cells as disclosed herein are administered to the subject not only help restore function to damaged or otherwise unhealthy tissues, but also facilitate remodeling of the damaged tissues.

To determine the suitability of cell compositions for therapeutic administration, the population of mature stem cell-derived beta cells can first be tested in a suitable animal model. At one level, cells are assessed for their ability to survive and maintain their phenotype in vivo. Cell compositions comprising mature stem cell-derived beta cells can be administered to immunodeficient animals (such as nude mice, or animals rendered immunodeficient chemically or by irradiation). Tissues are harvested after a period of regrowth, and assessed as to whether the administered cells or progeny thereof are still present.

This can be performed by administering cells that express a detectable label (such as green fluorescent protein, or β-galactosidase); that have been prelabeled (for example, with BrdU or [3H] thymidine), or by subsequent detection of a constitutive cell marker (for example, using human-specific antibody). The presence and phenotype of the administered population of mature stem cell-derived beta cells can be assessed by immunohistochemistry or ELISA using human-specific antibody, or by RT-PCR analysis using primers and hybridization conditions that cause amplification to be specific for human polynucleotides, according to published sequence data.

A number of animal models for testing diabetes are available for such testing, and are commonly known in the art, for example as disclosed in U.S. Pat. No. 6,187,991 which is incorporated herein by reference, as well as rodent models; NOD (non-obese mouse), BB_DB mice, KDP rat and TCR mice, and other animal models of diabetes as described in Rees et al, Diabet Med. 2005 April; 22(4):359-70; Srinivasan K, et al., Indian J Med. Res. 2007 March; 125(3):451-7; Chatzigeorgiou A, et al., In Vivo. 2009 March-April; 23(2):245-58, which are incorporated herein by reference.

In some embodiments, a population of mature stem cell-derived beta cells as disclosed herein may be administered in any physiologically acceptable excipient, where the mature stem cell-derived beta cells may find an appropriate site for replication, proliferation, and/or engraftment. In some embodiments, a population of mature stem cell-derived beta cells as disclosed herein can be introduced by injection, catheter, or the like. In some embodiments, a population of mature stem cell-derived beta cells as disclosed herein can be frozen at liquid nitrogen temperatures and stored for long periods of time, being capable of use on thawing. If frozen, a population of mature stem cell-derived beta cells will usually be stored in a 10% DMSO, 50% FCS, 40% RPMI 1640 medium. Once thawed, the cells may be expanded by use of growth factors and/or feeder cells associated with culturing mature stem cell-derived beta cells as disclosed herein.

In some embodiments, a population of mature stem cell-derived beta cells as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of mature stem cell-derived beta cells as disclosed herein will be adapted in accordance with the route and device used for administration. In some embodiments, a composition comprising a population of mature stem cell-derived beta cells can also comprise or be accompanied with one or more other ingredients that facilitate the engraftment or functional mobilization of the mature stem cell-derived beta cells. Suitable ingredients include matrix proteins that support or promote adhesion of the mature stem cell-derived beta cells, or complementary cell types, especially endothelial cells. In another embodiment, the composition may comprise resorbable or biodegradable matrix scaffolds.

In some embodiments, a population of mature stem cell-derived beta cells as disclosed herein may be genetically altered in order to introduce genes useful in insulin-producing cells such as pancreatic β cells, e.g. repair of a genetic defect in an individual, selectable marker, etc., or genes useful in selection against non-insulin-producing cells differentiated from at least one insulin-positive endocrine cell or precursor thereof or for the selective suicide of implanted mature stem cell-derived beta cells. In some embodiments, a population of mature stem cell-derived beta cells can also be genetically modified to enhance survival, control proliferation, and the like. In some embodiments a population of mature stem cell-derived beta cells as disclosed herein can be genetically altered by transfection or transduction with a suitable vector, homologous recombination, or other appropriate technique, so that they express a gene of interest. In one embodiment, a population of mature stem cell-derived beta cells is transfected with genes encoding a telomerase catalytic component (TERT), typically under a heterologous promoter that increases telomerase expression beyond what occurs under the endogenous promoter, (see International Patent Application WO 98/14592, which is incorporated herein by reference). In other embodiments, a selectable marker is introduced, to provide for greater purity of the population of mature stem cell-derived beta cells. In some embodiments, a population of mature stem cell-derived beta cells may be genetically altered using vector containing supernatants over a 8-16 h period, and then exchanged into growth medium for 1-2 days. Genetically altered mature stem cell-derived beta cells can be selected using a drug selection agent such as puromycin, G418, or blasticidin, and then recultured.

Gene therapy can be used to either modify a cell to replace a gene product, to facilitate regeneration of tissue, to treat disease, or to improve survival of the cells following implantation into a subject (i.e. prevent rejection).

In an alternative embodiment, a population of mature stem cell-derived beta cells as disclosed herein can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are cells that are genetically altered to express one or more growth factors of various types, such as somatostatin, glucagon, and other factors.

Many vectors useful for transferring exogenous genes into target mature stem cell-derived beta cells as disclosed herein are available. The vectors may be episomal, e.g. plasmids, virus derived vectors such as cytomegalovirus, adenovirus, etc., or may be integrated into the target cell genome, through homologous recombination or random integration, e.g. retrovirus derived vectors such MMLV, HIV-1, ALV, etc. In some embodiments, combinations of retroviruses and an appropriate packaging cell line may also find use, where the capsid proteins will be functional for infecting the mature stem cell-derived beta cells as disclosed herein. Usually, mature stem cell-derived beta cells and virus will be incubated for at least about 24 hours in the culture medium. In some embodiments, the mature stem cell-derived beta cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PA12 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g. AKR env, are capable of infecting most mammalian cell types, except murine cells. In some embodiments, the vectors may include genes that must later be removed, e.g. using a recombinase system such as Cre/Lox, or the cells that express them destroyed, e.g. by including genes that allow selective toxicity such as herpesvirus TK, Bc1-Xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

In one aspect of the present invention, a population of mature stem cell-derived beta cells as disclosed herein is suitable for administering systemically or to a target anatomical site. A population of mature stem cell-derived beta cells can be grafted into or nearby a subject's pancreas, for example, or may be administered systemically, such as, but not limited to, intra-arterial or intravenous administration. In alternative embodiments, a population of mature stem cell-derived beta cells of the present invention can be administered in various ways as would be appropriate to implant in the pancreatic or secretory system, including but not limited to parenteral, including intravenous and intraarterial administration, intrathecal administration, intraventricular administration, intraparenchymal, intracranial, intracisternal, intrastriatal, and intranigral administration. Optionally, a population of mature stem cell-derived beta cells is administered in conjunction with an immunosuppressive agent.

In some embodiments, a population of mature stem cell-derived beta cells can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. A population of mature stem cell-derived beta cells can be administered to a subject the following locations: clinic, clinical office, emergency department, hospital ward, intensive care unit, operating room, catheterization suites, and radiologic suites.

In other embodiments, a population of mature stem cell-derived beta cells is stored for later implantation/infusion. A population of mature stem cell-derived beta cells may be divided into more than one aliquot or unit such that part of a population of mature stem cell-derived beta cells is retained for later application while part is applied immediately to the subject. Moderate to long-term storage of all or part of the cells in a cell bank is also within the scope of this invention, as disclosed in U.S. Patent Application Serial No. 20030054331 and Patent Application No. WO03024215, and is incorporated by reference in their entireties. At the end of processing, the concentrated cells may be loaded into a delivery device, such as a syringe, for placement into the recipient by any means known to one of ordinary skill in the art.

In some embodiments a population of mature stem cell-derived beta cells can be applied alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. In some embodiments, a population of mature stem cell-derived beta cells may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, in some embodiments, a population of mature stem cell-derived beta cells could be combined with a gene encoding pro-angiogenic growth factor(s). Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid adeno-associated virus. Cells could be implanted along with a carrier material bearing gene delivery vehicle capable of releasing and/or presenting genes to the cells over time such that transduction can continue or be initiated. Particularly when the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 2002/0182211, which is incorporated herein by reference. In one embodiment, an immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the cardiovascular stem cells of the invention.

Pharmaceutical compositions comprising effective amounts of a population of mature stem cell-derived beta cells are also contemplated by the present invention. These compositions comprise an effective number of mature stem cell-derived beta cells, optionally, in combination with a pharmaceutically acceptable carrier, additive or excipient. In certain aspects of the present invention, a population of mature stem cell-derived beta cells is administered to the subject in need of a transplant in sterile saline. In other aspects of the present invention, a population of mature stem cell-derived beta cells is administered in Hanks Balanced Salt Solution (HBSS) or Isolyte S, pH 7.4. Other approaches may also be used, including the use of serum free cellular media. In one embodiment, a population of mature stem cell-derived beta cells is administered in plasma or fetal bovine serum, and DMSO. Systemic administration of a population of mature stem cell-derived beta cells to the subject may be preferred in certain indications, whereas direct administration at the site of or in proximity to the diseased and/or damaged tissue may be preferred in other indications.

In some embodiments, a population of mature stem cell-derived beta cells can optionally be packaged in a suitable container with written instructions for a desired purpose, such as the reconstitution or thawing (if frozen) of a population of mature stem cell-derived beta cells prior to administration to a subject.

In one embodiment, an isolated population of mature stem cell-derived beta cells as disclosed herein is administered with a differentiation agent. In one embodiment, the mature stem cell-derived beta cells are combined with the differentiation agent to administer into the subject. In another embodiment, the cells are administered separately to the subject from the differentiation agent. Optionally, if the cells are administered separately from the differentiation agent, there is a temporal separation in the administration of the cells and the differentiation agent. The temporal separation may range from about less than a minute in time, to hours or days in time. The determination of the optimal timing and order of administration is readily and routinely determined by one of ordinary skill in the art.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the disclosure. Further, all patents, patent applications, and publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents

### EXAMPLES

### Example 1—Manipulation of the PI3K/Akt/mTOR Pathway to Enhance Maturation of Stem Cell-Derived Beta Cells

In recent years, improved knowledge has enabled a more complete understanding of the intrinsic transcriptional programs that lead to cell and tissue development in utero and the ability to generate many cell types, including beta cells, from human pluripotent stem cells has advanced rapidly (Hrvatin et al., 2014; Pagliuca et al., 2014; Sneddon et al., 2018). Nevertheless, because there is a lack of knowledge about functional maturation, differentiated tissues derived from stem cells often resemble fetal tissues and lack full physiological function (Henquin and Nenquin, 2018; Hrvatin et al., 2014).

Functional maturation is generally studied as an intrinsic part of the differentiation program rather than as being extrinsically regulated by factors from the environment, such as nutrients. Yet it is known that cells sense extrinsic signals and coordinate cellular choices accordingly. For example, cells modulate their metabolism and growth in response to nutrients and growth factors via regulation of the mechanistic target of rapamycin complex 1 (mTORC1) signaling. mTORC1 kinase activity controls cellular growth and metabolism by activating anabolic processes and repressing catabolic ones (Sancak et al., 2010; Sancak et al., 2008; Saxton and Sabatini, 2017). Several cytosolic proteins have been identified that signal the availability of nutrients to mTORC1 (Saxton and Sabatini, 2017). Of these nutrient-sensitive regulators, Sestrin1 and Sestrin2 are leucine-regulated inhibitors of mTORC1. Leucine stimulation relieves the inhibition by the Sestrins and thereby allows mTORC1 activation (Wolfson et al., 2016; Wolfson and Sabatini, 2017; Wyant et al., 2017).

In this work, the possibility that sensing environmental nutrients contributes to tissue maturation was investigated. The findings show an unappreciated role for nutrient sensing by mTORC1 in the functional maturation of pancreatic beta cells. A change in the regulation of mTORC1 signaling by nutrients, from activity in immature beta cells that is amino acid-dependent and glucose-independent to mTORC1 activity in mature beta cells that is dependent on both glucose and amino acids, is discussed. In fetal beta cells, which develop in an amino acid-rich environment in utero, mTORC1 is constitutively active. The environment of mature beta cells, however, contains lower levels of amino acids and oscillating levels of glucose. Therefore, mTORC1 activity in mature beta cells is dynamic, periodically becoming activated following feeding and inhibited during fasting. Furthermore, mice harboring a beta cell-specific deletion of Sestrin1 and Sestrin2 are used to validate that disrupting the regulation of mTORC1 by environmental nutrients in mature beta cells reverts beta cells to an immature functional state. Finally, it is demonstrated that immature human stem cell-derived beta cells similarly undergo a shift in mTORC1 nutrient sensitivity and function, from constitutive to glucose-dependent insulin secretion, following an environmental switch from amino acid-rich to amino acid-poor culture conditions (e.g., upon transplant).

### Results

mTORC1 Activation in Mature Beta Cells is Glucose-Dependent

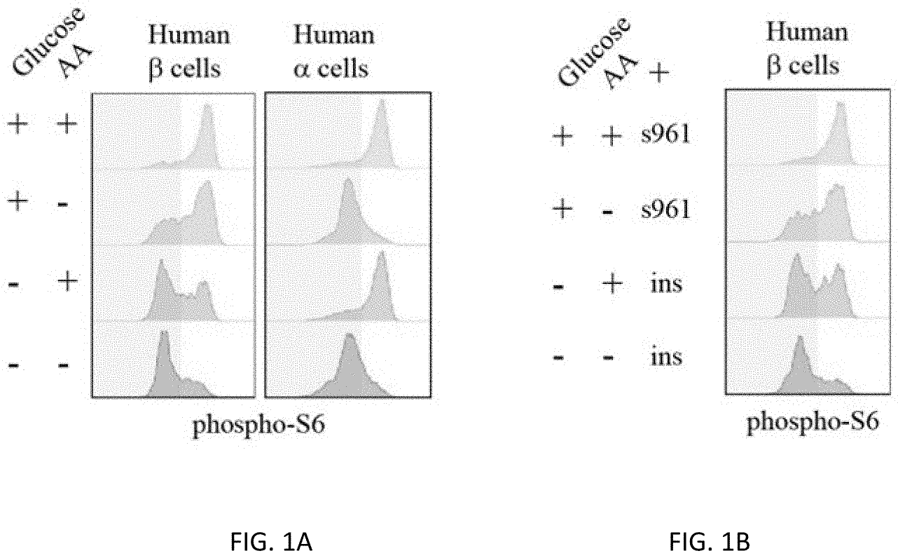

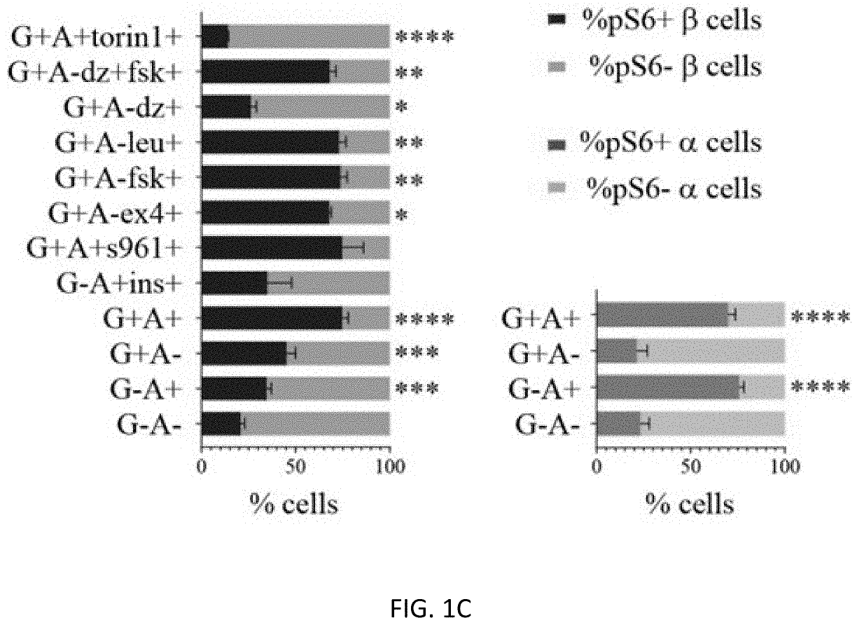

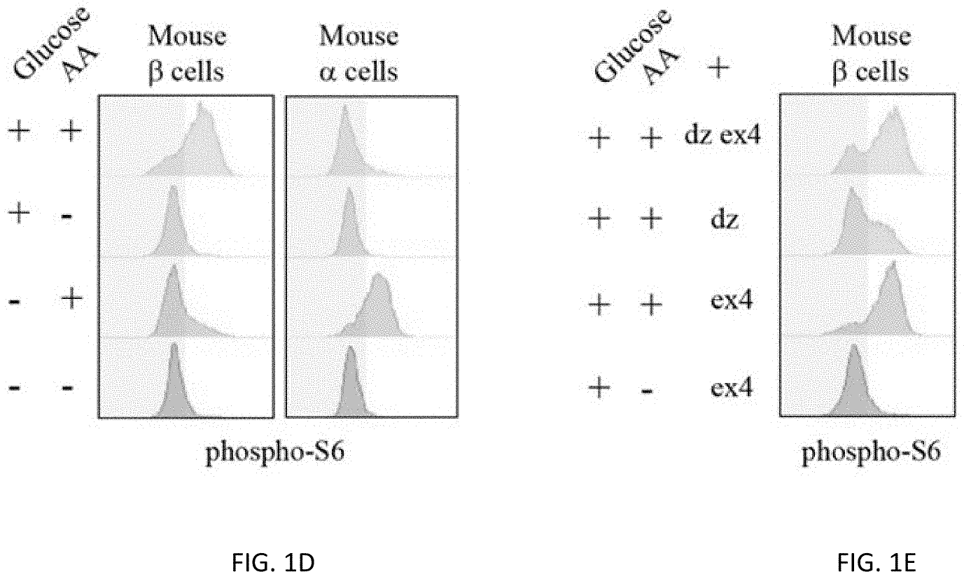

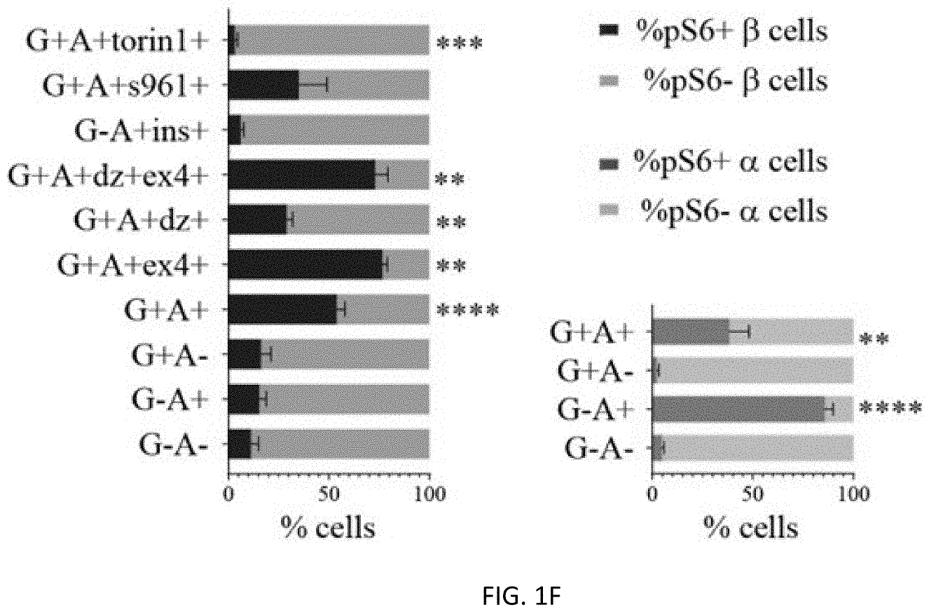

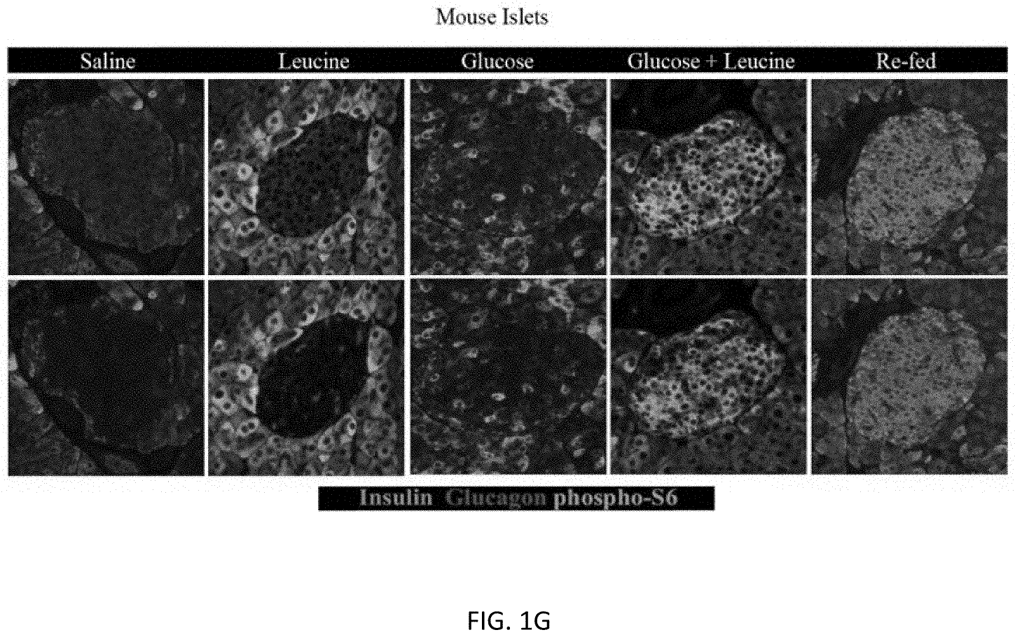

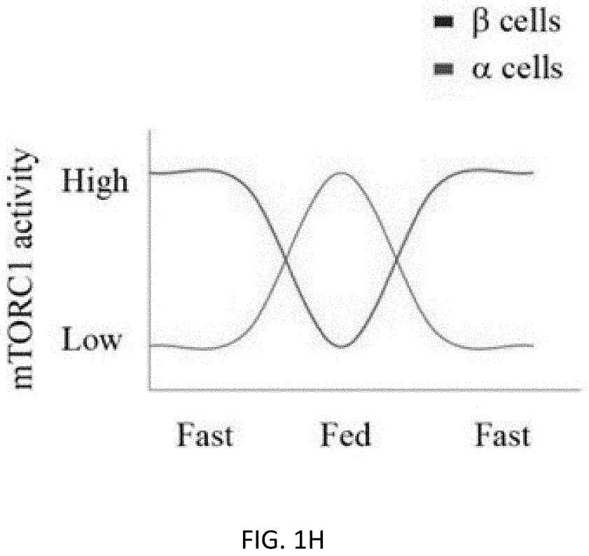

Insulin-expressing beta cells appear around embryonic day 13.5 (E13.5) in mice and week 8 and 9 post-conception in humans (Pan and Wright, 2011; Slack, 1995), but glucose-stimulated insulin secretion (GSIS) has been observed only days after birth (Blum et al., 2012). Since a major change from embryonic to postnatal physiology is nutrient consumption, it was predicted that nutrients and nutrient sensing by mTORC1 could have a role in the transition from immature to mature insulin secretion by beta cells. To explore that, the nutrient sensitivity and dynamics of mTORC1 in isolated mature human and mouse islets was first examined. Differential effects by nutrients on the percentage of beta cells that activate mTORC1 were found, as measured by phosphorylation of rpS6 (p-S6), a downstream target and established marker of mTORC1 kinase activity. Amino acids only are not sufficient to activate mTORC1, activating ˜25% of the cells, slightly more than the basal activity of mTORC1 in mature beta cells. Generally, glucose alone induces activation of mTORC1 in a higher percentage of beta cells, while addition of both glucose and amino acids generates a robust activation (˜80%), indicating a greater dependence on glucose for mTORC1 activation in mature beta cells and a requirement of both amino acids and glucose for full activity (FIGS. 1A and 1C).

Whether a similar pattern of mTORC1 responsiveness to nutrients occurs in mature mouse islets was also tested. It was found that only glucose in the presence of amino acids induces robust mTORC1 activation, comparable to that seen in human beta cells (FIGS. 1D and 1F).

Human and mouse alpha cells showed a different nutrient sensitivity and were fully induced by amino acids—glucose does not promote alpha cell mTORC1 activity, and even overrides the inductive effect of amino acids to suppresses it in murine alpha cells (FIGS. 1A, 1C, 1D and 1F).

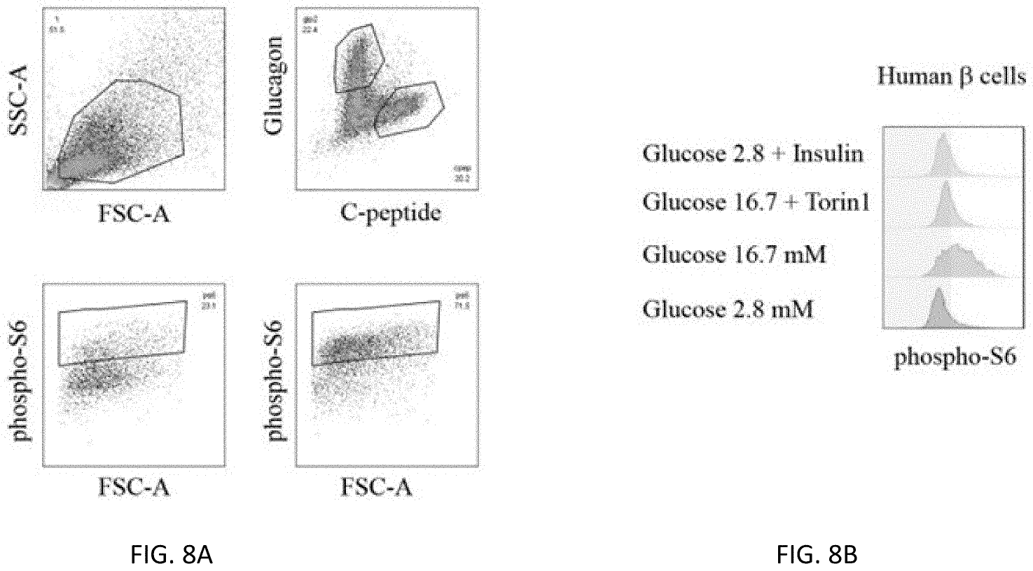

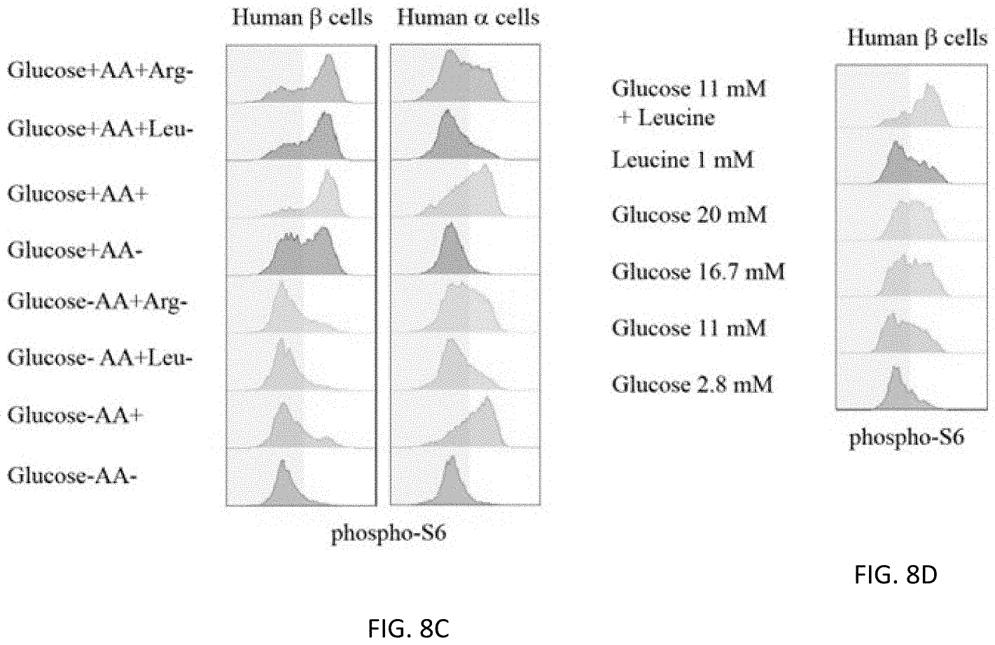

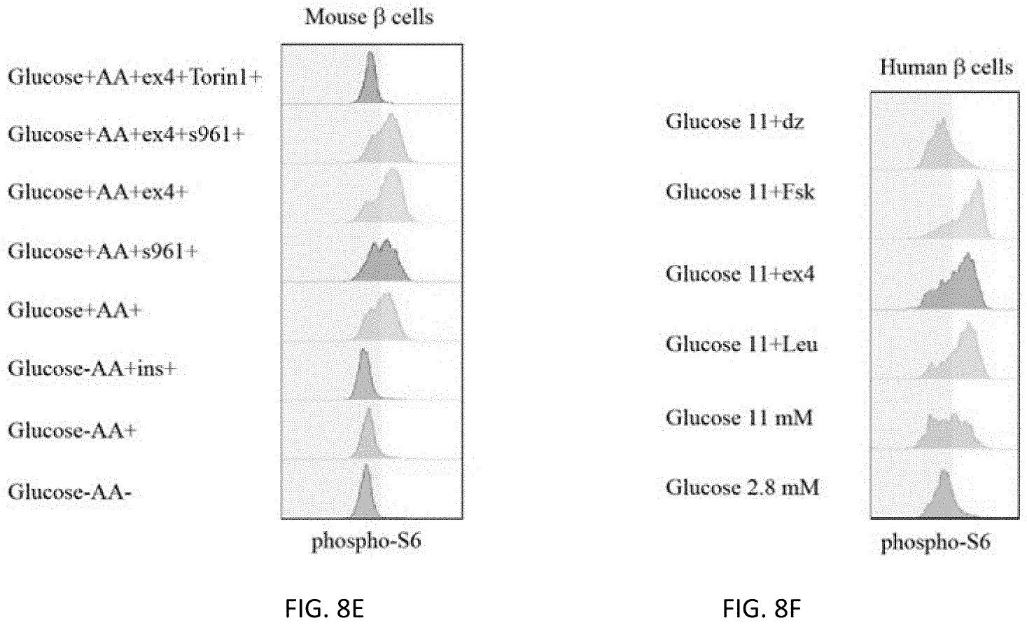

Overall, these results indicate that in mature beta cells mTORC1 is responsive to glucose in the presence of amino acids, and in alpha cells to amino acids alone. Among amino acids, leucine has been implicated in mTORC1 activation. It was found that leucine is sufficient to fully activate the beta cell mTORC1 pathway in the presence of glucose (FIG. 1C and FIG. 8). In fact, while addition of increasing glucose alone (from 8-20 mM) dose-dependently enhances mTORC1 activity in beta cells, including a low concentration of leucine results in full mTORC1 activation, even in intermediate glucose concentrations (8-11 mM). Leucine levels therefore play a role in establishing the range of glucose concentrations capable of regulating mTORC1 activity in beta cells. Further, by removing leucine it was found that beta cells require leucine to fully activate mTORC1 in high glucose concentrations (FIG. 8). This suggests an essential role for leucine in the regulation of mTORC1 in mature human beta cells.

Interestingly it was found that mTORC1 activity in beta cells is increased by stimulation with amplifiers of insulin secretion, such as Exendin-4 and Forskolin (a Glp1 analogue and PKA activator, respectively), demonstrating a positive correlation between insulin secretion and mTORC1 activation (FIGS. 1C, 1E, 1F and FIG. 8). Indeed, pharmacologically suppressing insulin secretion with diazoxide dramatically reduces mTORC1 activity in beta cells, even in the presence of glucose and amino acids, and this is rescued by adding Forskolin or Exendin-4 (FIG. 1C and FIG. 8). However, mTORC1 reaction to nutrients is not through secreted insulin, as blocking insulin receptor signaling did not affect mTORC1 activation in the presence of glucose. Further, in the absence of glucose, neither stimulation with insulin nor stimulation with insulin and amino acids was able to activate mTORC1 in beta cells, indicating that insulin receptor signaling does not mediate the effects of glucose on mTORC1 signaling in beta cells (FIGS. 1B, 1C and FIG. 8). These results suggest that intracellular crosstalk in which calcium flux downstream of the ATP-sensitive potassium channels is required for mTORC1 activation.

mTORC1 is Transiently Activated in Mature Beta Cells Following Feeding

The regulation by glucose and insulin secretion suggests that mTORC1 signaling in beta cells in vivo is mostly inhibited and activated only transiently following feeding. To assess this, mTORC1 activation was analyzed in the islets of fasted mice upon injection with glucose and/or leucine. Indeed, staining for p-S6 shows that mTORC1 is strongly suppressed in beta cells after an overnight fast. Moreover, supplementation of leucine is not sufficient to activate the pathway, whereas glucose injection partially activates mTORC1 within the islets. Injection of glucose and leucine together activates mTORC1 in most beta cells similarly to feeding, (FIG. 1G), consistent with the responses of isolated murine islets to nutrient treatments in vitro (FIG. 1D). Altogether, these results indicate that mTORC1 activation in alpha and beta cells is highly regulated by nutrients. Alpha cells activate the pathway in the presence of amino acids (likely supplied from autophagy) and the absence of glucose (e.g., during fasting), whereas beta cells activate mTORC1 merely following glucose consumption and insulin secretion during feeding (FIG. 1H).

mTORC1 Signaling in Immature Beta Cells is Independent of Glucose and Constitutively Active In Utero

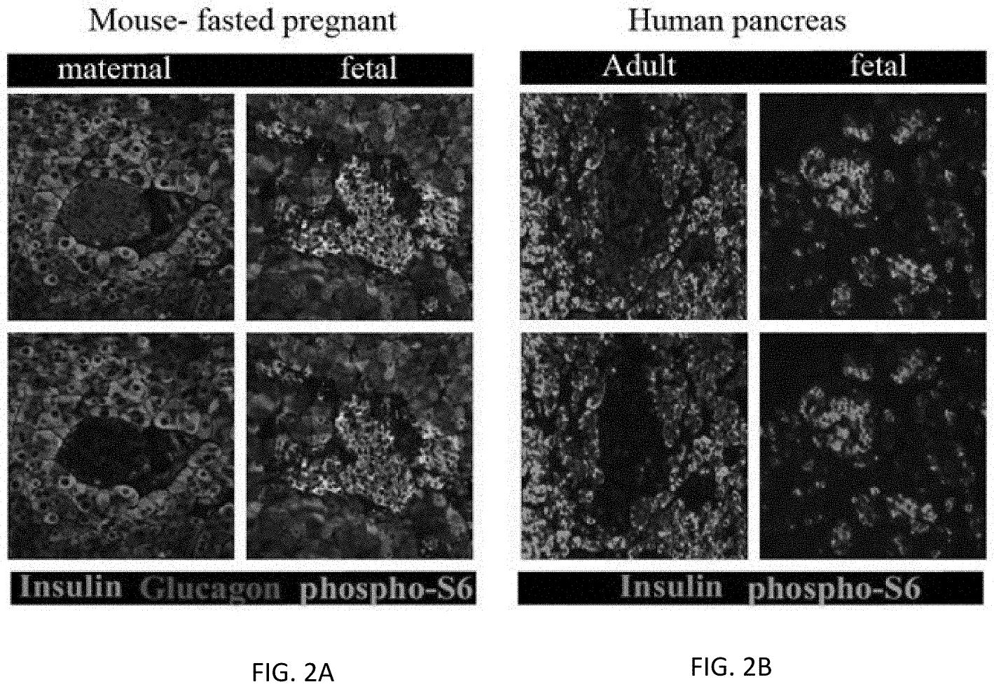

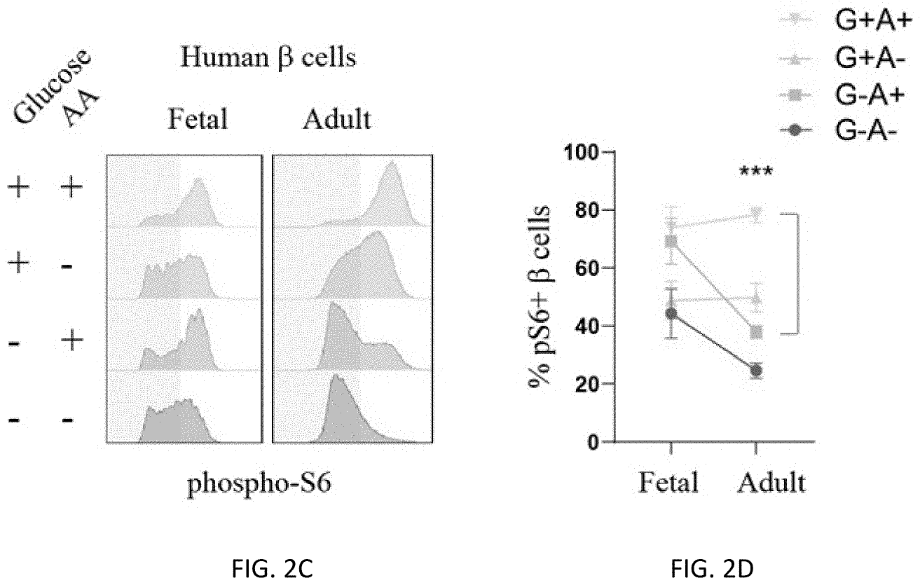

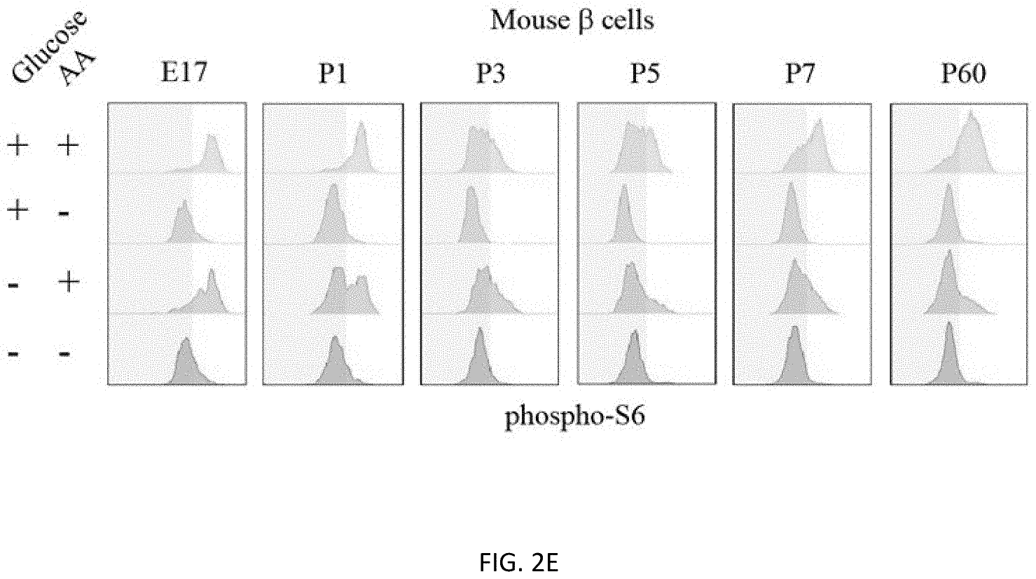

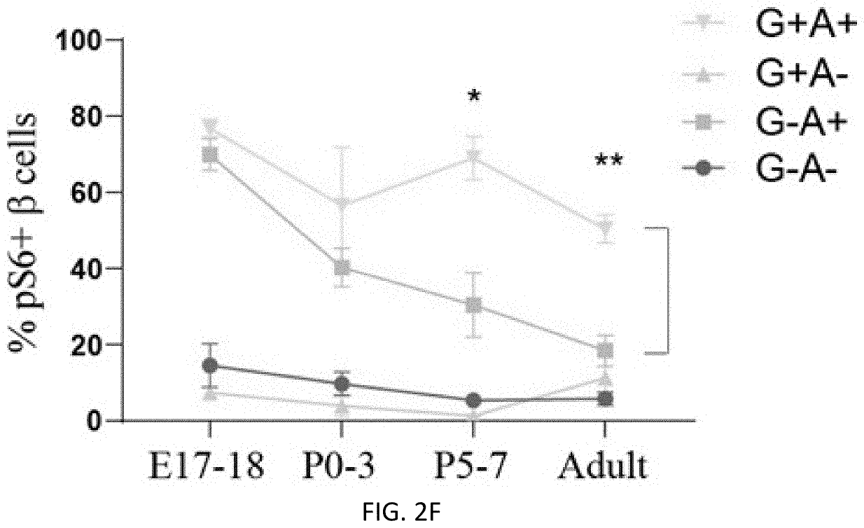

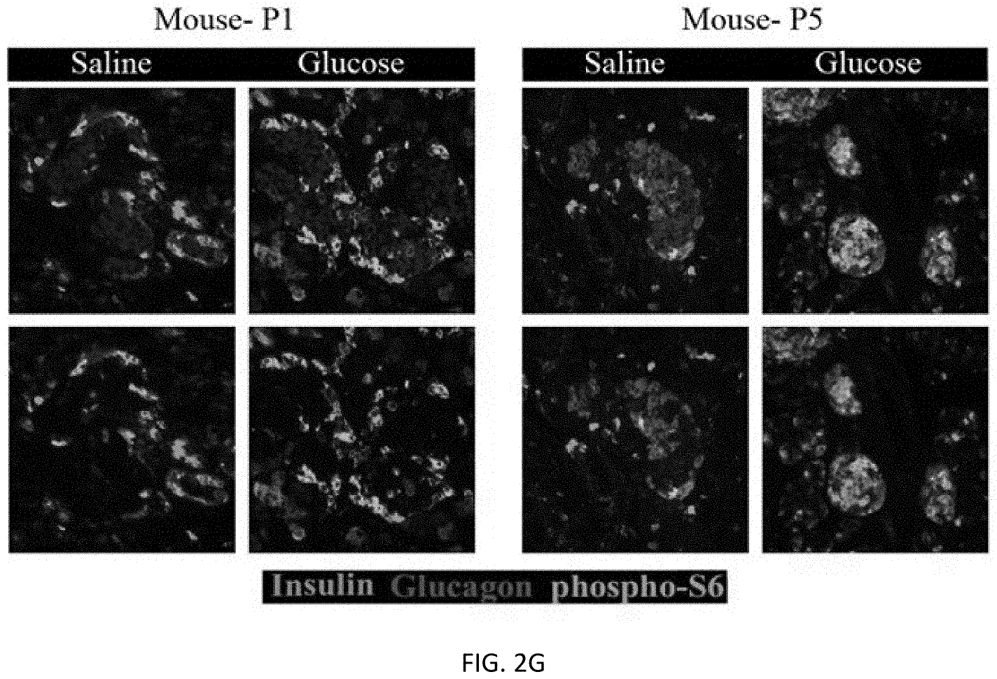

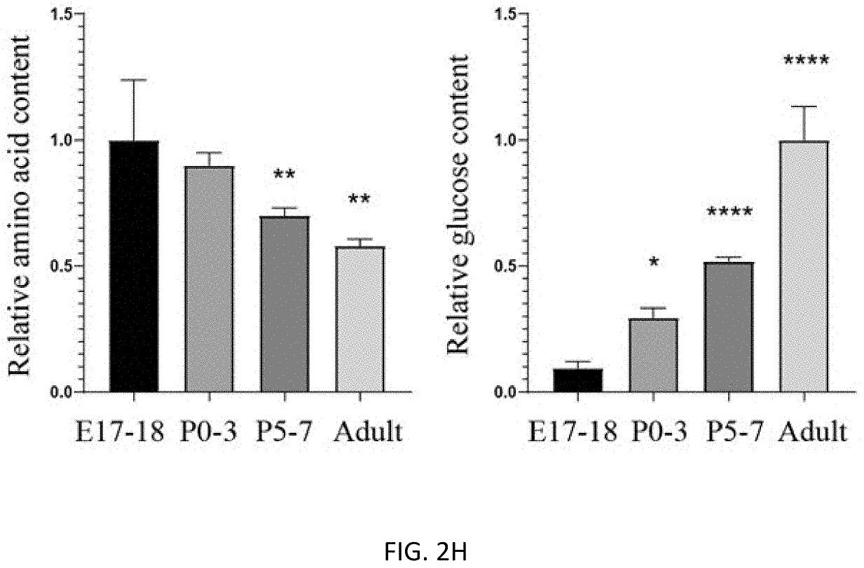

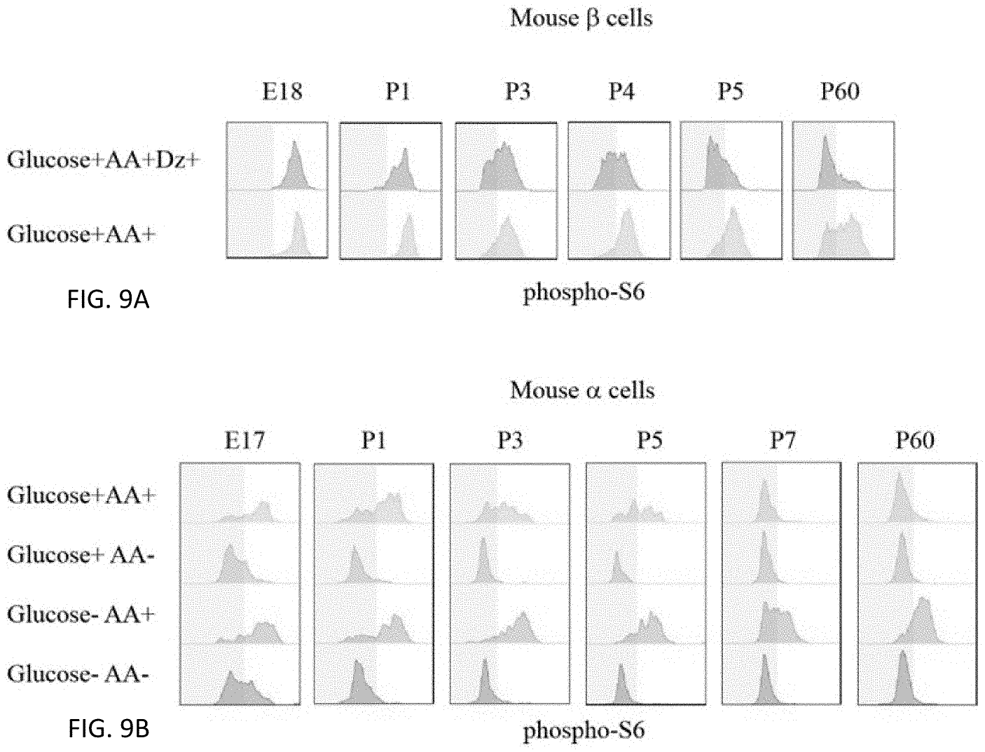

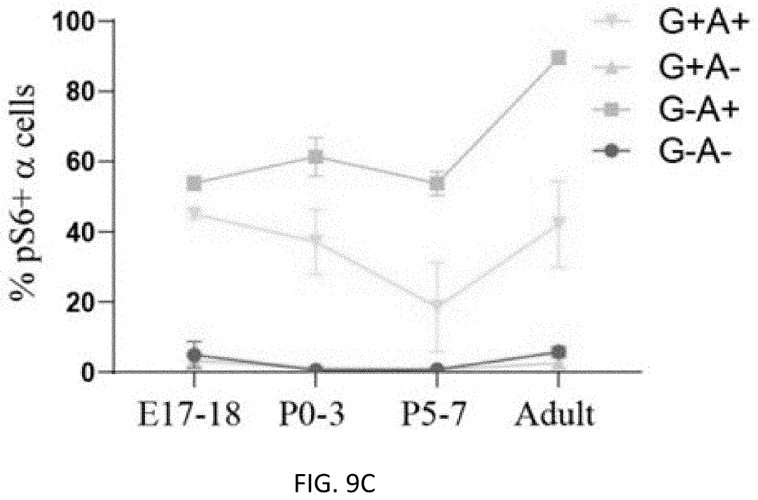

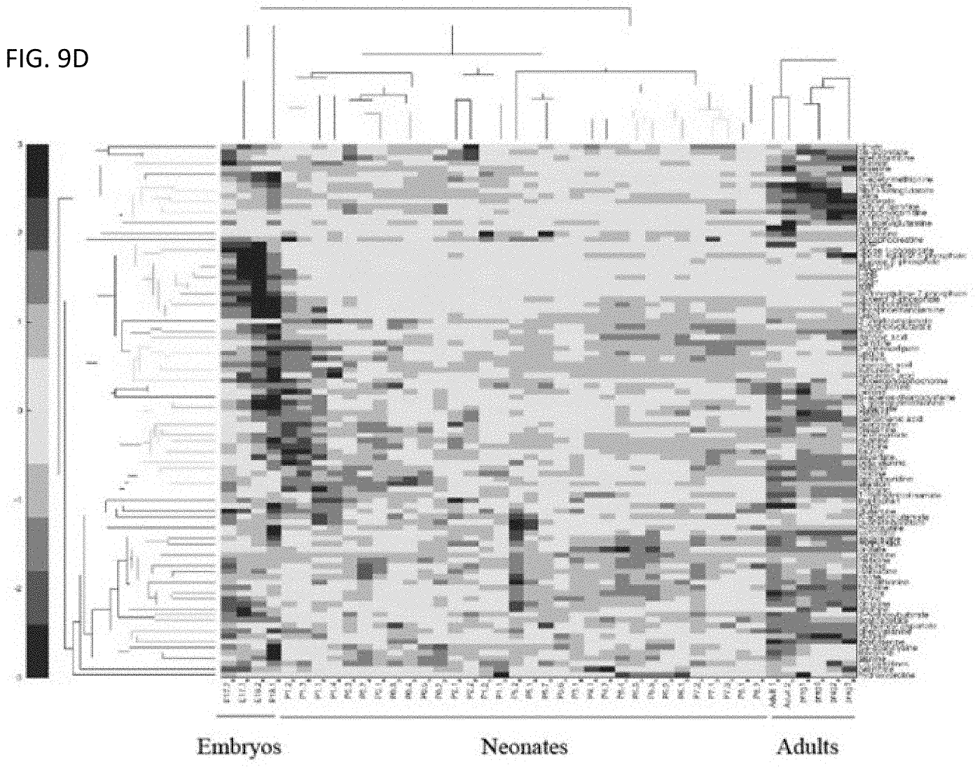

To explore mTORC1 signaling dynamics in immature beta cells, fetal pancreatic tissue was collected from embryonic mice. Surprisingly, whereas pancreatic beta cells of the fasted pregnant females had low mTORC1 activity, the pathway was strongly activated in the beta cells of the embryos (FIG. 2A). Similar mTORC1 hyperactivity in fetal beta cells is also evident when human fetal and adult pancreatic tissue is compared (FIG. 2B). Two possible explanations for this difference are that the placental blood supply maintains high fetal nutrient levels independent of maternal feeding status, or that mTORC1 signaling in fetal beta cells responds to different nutrients as compared to mature beta cells. Hence, to test whether mTORC1 nutrient sensitivity changes during beta cell maturation, mTORC1 activity was compared between isolated embryonic and mature human islets ex vivo in identical nutrient conditions. Remarkably, it was found that mTORC1 activity in immature beta cells is completely independent of glucose stimulation and requires only amino acids for full activation, in contrast to mature beta cells which require glucose for full activation, in both human (FIGS. 2C and 2D) and mouse islets (FIGS. 2E and 2F). Moreover, islets from neonatal mice were isolated and the shift from immature to mature nutrient responsiveness was observed occurring gradually over the first postnatal week. As the neonates aged from postnatal day 1 to day 7, their beta cells developed a progressively increased requirement for glucose to activate mTORC1, evidenced by the gradual decreased induction of mTORC1 activity in response to amino acids alone (FIGS. 2E and 2F). It was also observed that in immature, amino acid-responsive, beta cells mTORC1 signaling is not coupled to the insulin secretion pathway, since inhibiting insulin secretion with diazoxide does not affect mTORC1 activation (FIG. 9). The opposite maturation process occurs in alpha cells during the first week of life, wherein glucose increasingly inhibits mTORC1 activation (FIG. 9).

To test the nutrient sensitivity of mTORC1 in beta cells in vivo, glucose was injected into neonates and their pancreata was dissected. In the first day after birth mTORC1 activity in beta cells is dramatically reduced compared to the final day of gestation. In contrast to mature beta cells, injection of glucose does not activate mTORC1 in immature beta cells of postnatal day 1 neonates. At postnatal day 5, however, beta cells activate the mTORC1 pathway in response to glucose, in agreement with the ex vivo results (FIGS. 2G and 2E, respectively).

To test the levels of glucose and amino acids in the embryonic serum the metabolite profiles of fetal and maternal plasma were compared. Surprisingly, the composition of amino acids and glucose in the plasma of the embryos and their mothers was strikingly different. In general, levels of amino acids and amino acid-derived metabolites in the fetal plasma were higher and glucose was much lower as compared to maternal plasma. This excludes the possibility that fetal beta cells have high mTORC1 activity due to an excess of both glucose and amino acids in the in utero environment (FIG. 2H).

Together these results demonstrate that mTORC1 in embryonic immature beta cells is constantly activated, because amino acids are sufficient to activate mTORC1 and because they are growing in an amino acid-rich environment in utero. Upon birth, the mTORC1 pathway in beta cells becomes dependent on glucose. Therefore, the change from a constant nutrient supply to discontinuous feedings results in fluctuating blood glucose concentrations, which further results in the dynamic activation and inhibition of the mTORC1 pathway.

Robust mTORC1 Activation in Response to Amino Acid Stimulation Impairs SC-Beta Cell Function

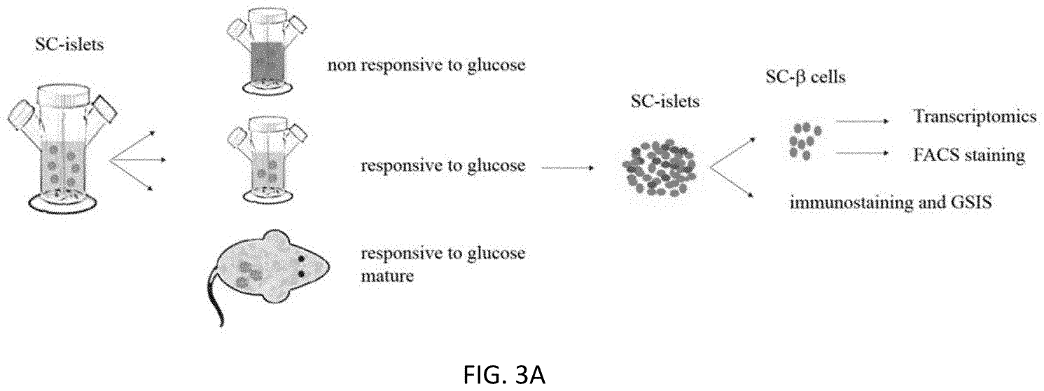

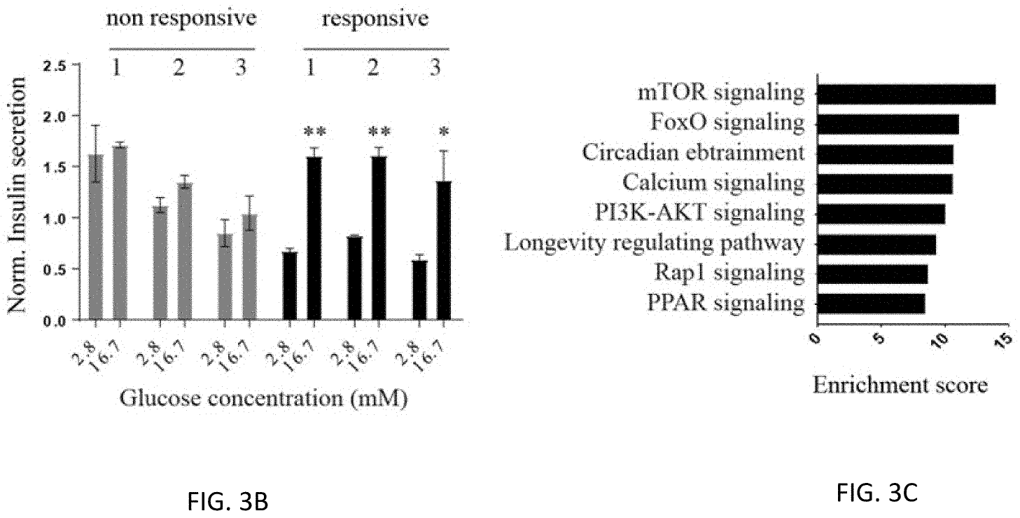

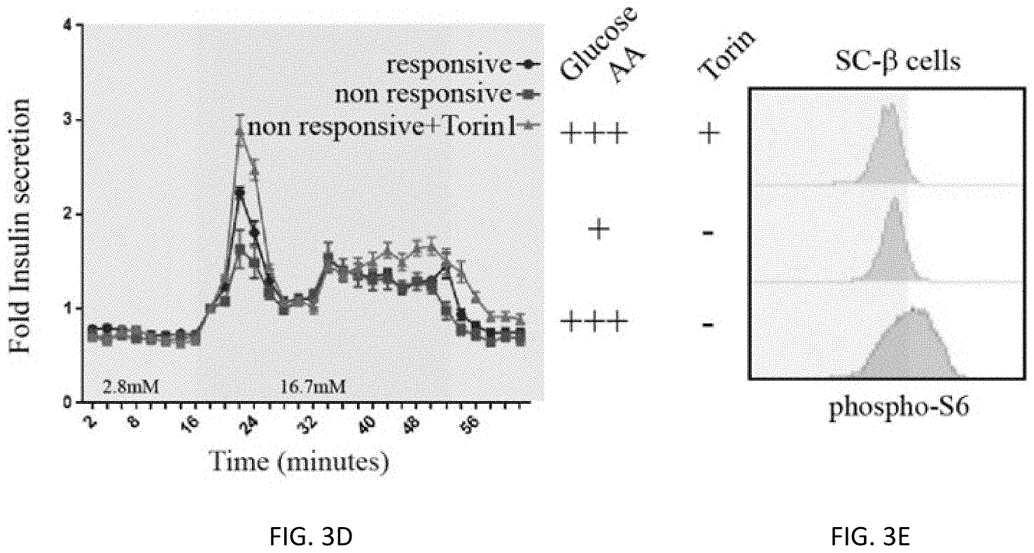

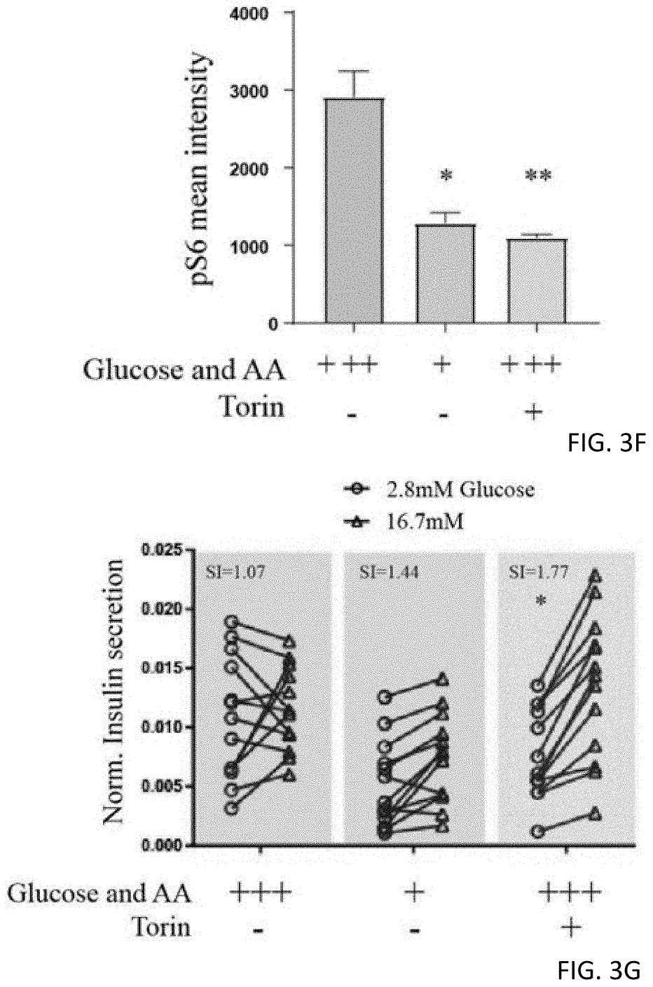

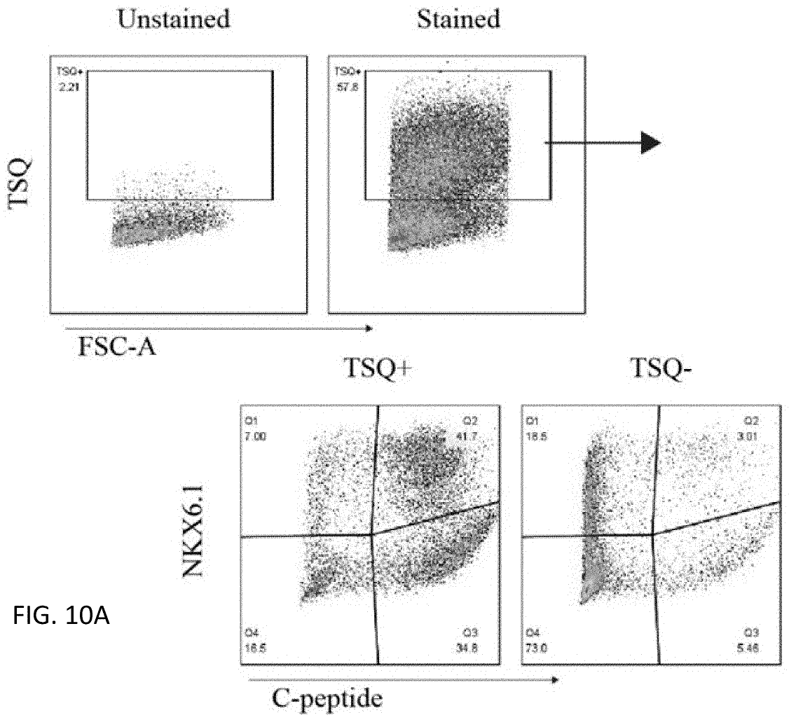

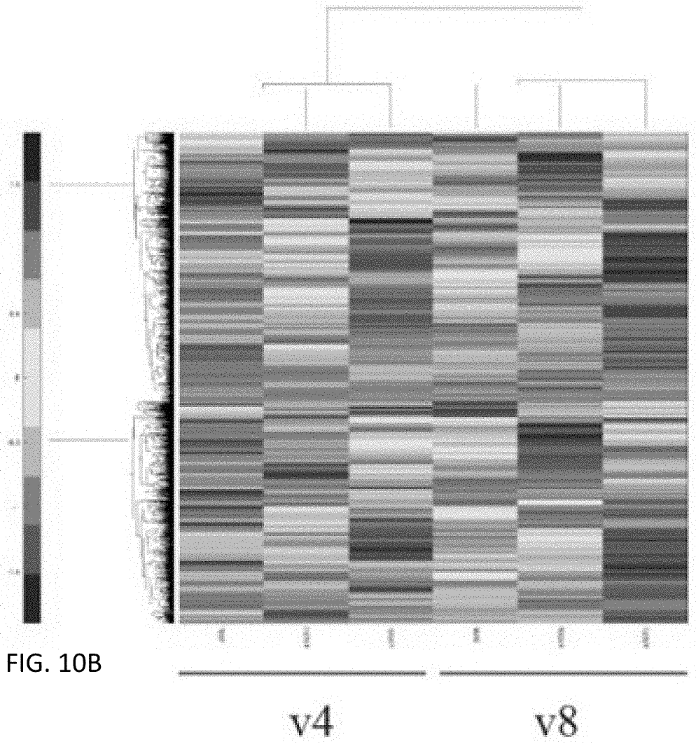

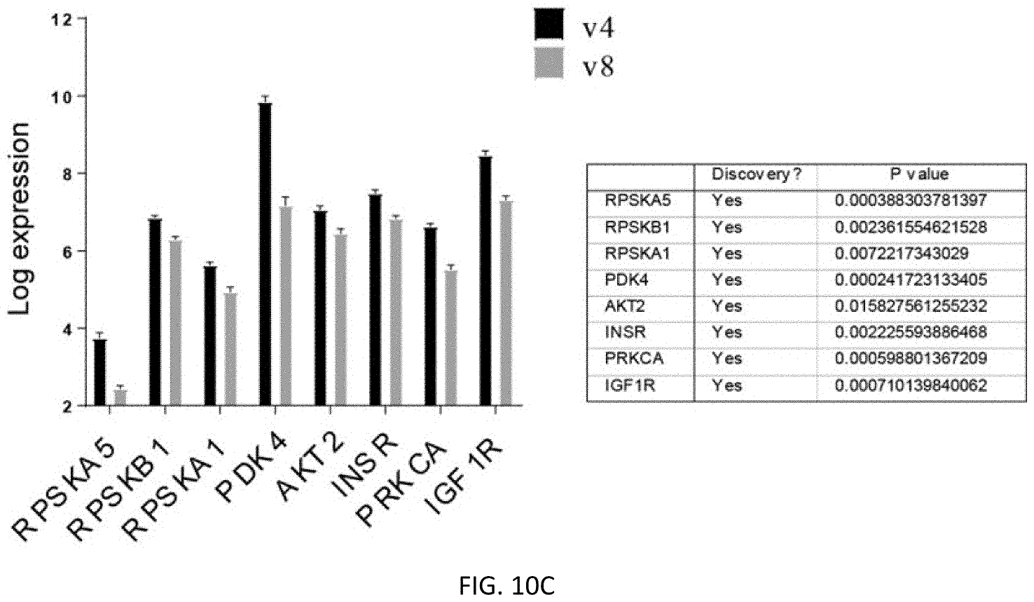

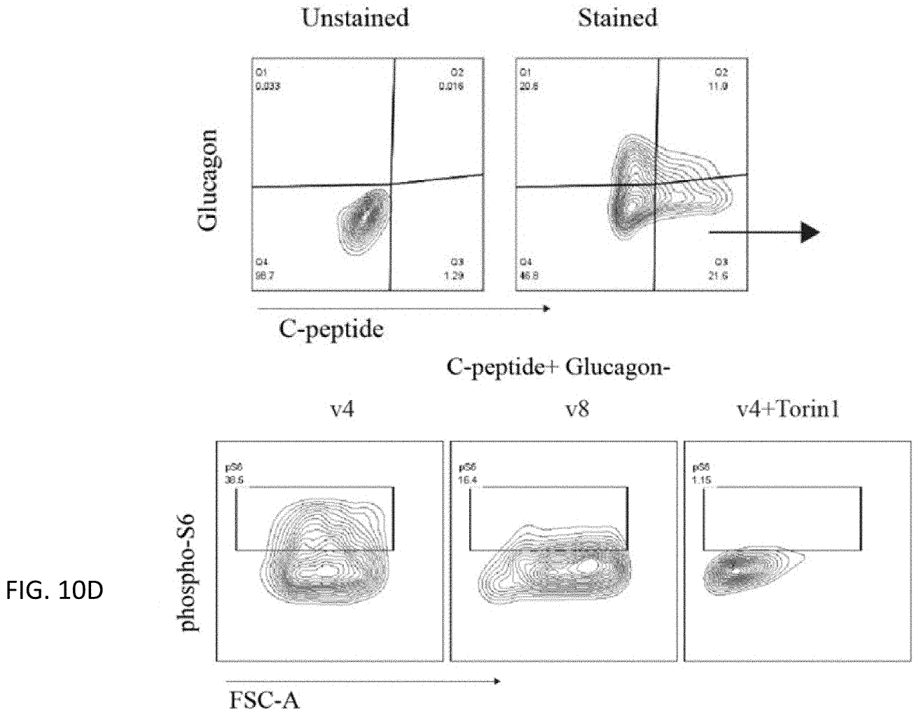

Next, it was sought to understand the relationship between mTORC1 activation and insulin secretion using differentiated human induced pluripotent stem cell-derived beta cells (SC-beta). This relationship was relevant for two specific reasons. First, while differentiation protocols for SC-beta cells can successfully generate functional insulin-secreting SC-beta cells, the resulting cells do not fully achieve the degree of glucose-responsive insulin secretion seen in primary beta cells. Second, several versions of the differentiation protocol have been developed which generate markedly different glucose-response capacities. Comparing the differences in these protocols can thus help to understand how beta cells acquire responsiveness to glucose. There was therefore a focus on two growing conditions of differentiated SC-beta cells, an earlier version that produces an SC-beta cell whose insulin secretion is not glucose-responsive, and a more recent version of the protocol which produces SC-beta cells that steadily secrete more insulin in high (16.7 mM) compared to low (2.8 mM) glucose concentrations (FIGS. 3A and 3B). Importantly, the two conditions produce a similar number of SC-beta cells (20-40% of total cells), and differ primarily in the culture media of the fully differentiated cells in the final stage of the protocol (i.e., that is in the nutritional environment of the cells). Transcriptional analyses, using mRNA from enriched populations of FACS-sorted SC-beta cells, revealed substantial differences between insulin-expressing cells generated by the two conditions (FIG. 10). Surprisingly, in spite of their functional profile, the cells grown under GSIS-permissive conditions did not express genes considered to be markers of maturation such as MAFA, UCN3, FLTP and SIX2/3 (Arda et al., 2016; Bader et al., 2016; Blum et al., 2012; Nishimura et al., 2015). This suggests that these markers are not directly related with actual acquisition of glucose responsiveness.

Notably, pathway enrichment analysis revealed that target genes of the mTORC1 pathway are highly enriched in glucose-nonresponsive SC-beta cells, suggesting a negative role for mTORC1 signaling in the regulation of glucose-stimulated insulin secretion (FIG. 3C and FIG. 10). Correspondingly, enhanced mTORC1 signaling was found in the nonresponsive SC-beta cells as determined by increased phosphorylation of p-S6, which can be repressed by treatment with the mTOR inhibitor Torin1 (FIG. 10). To test if mTORC1 activity negatively affects glucose responsiveness of SC-beta cells, nonresponsive SC-beta cells were treated with Torin1 for 48 hours and a dynamic GSIS assay was conducted. Indeed, inhibiting the pathway induced insulin secretion in response to glucose (FIG. 3D). To directly test the effects of nutrients on mTORC1 activity and GSIS, SC-beta cell clusters were cultured in high or low concentrations of glucose and amino acids. It was found that culturing SC-beta cells in a high nutrient environment evokes strong mTORC1 activity that can be repressed by Torin1 (FIGS. 3E and 3F). Remarkably, reducing mTORC1 activity in SC-beta cells by growing them in nutrient-poor conditions or with Torin1 increases their GSIS (FIG. 3G).

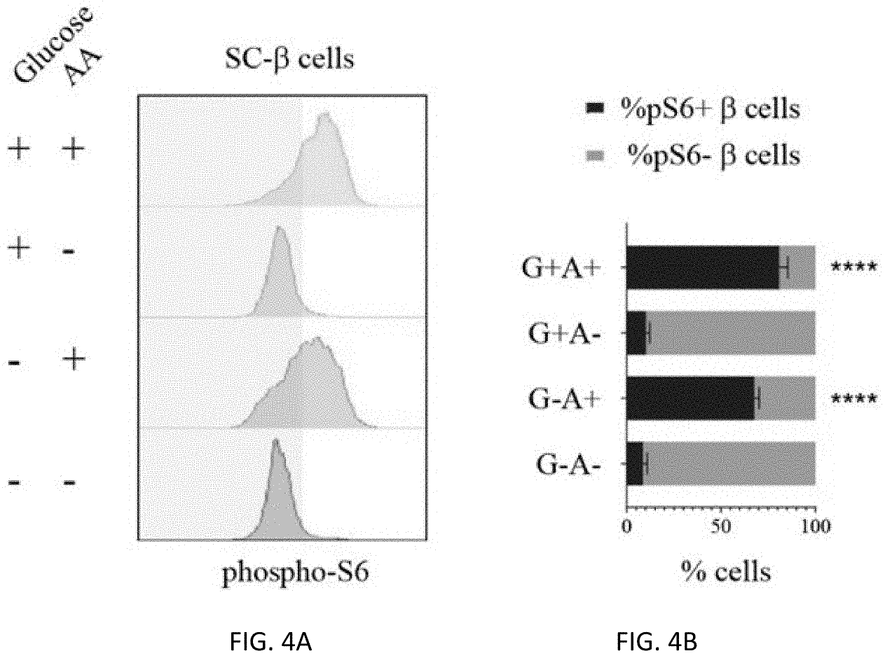

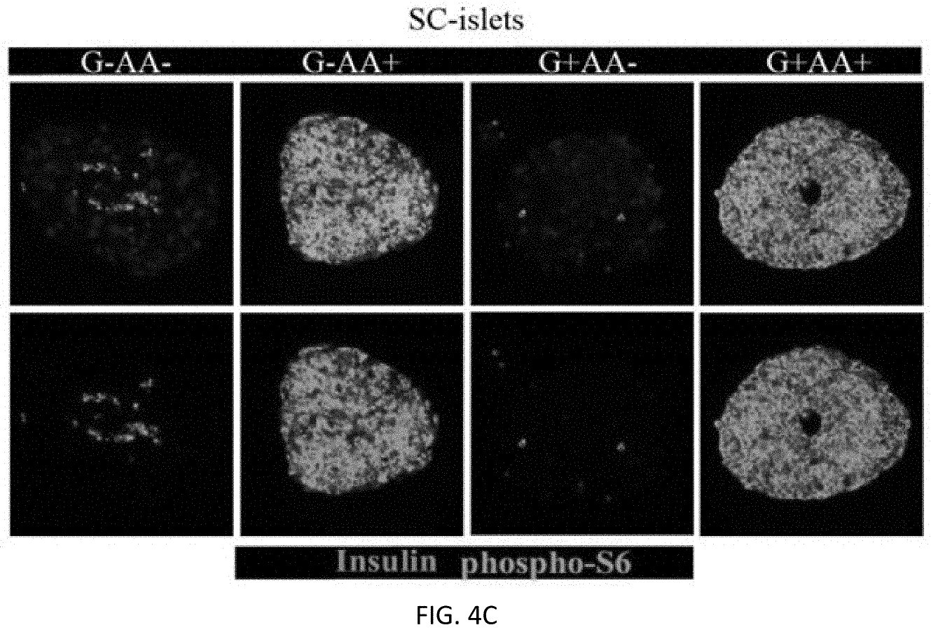

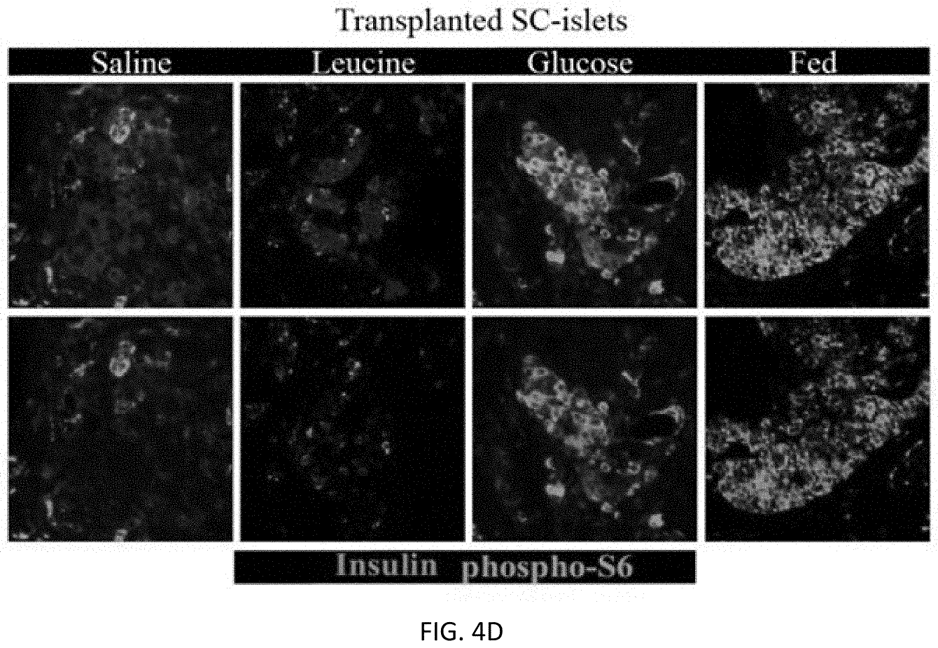

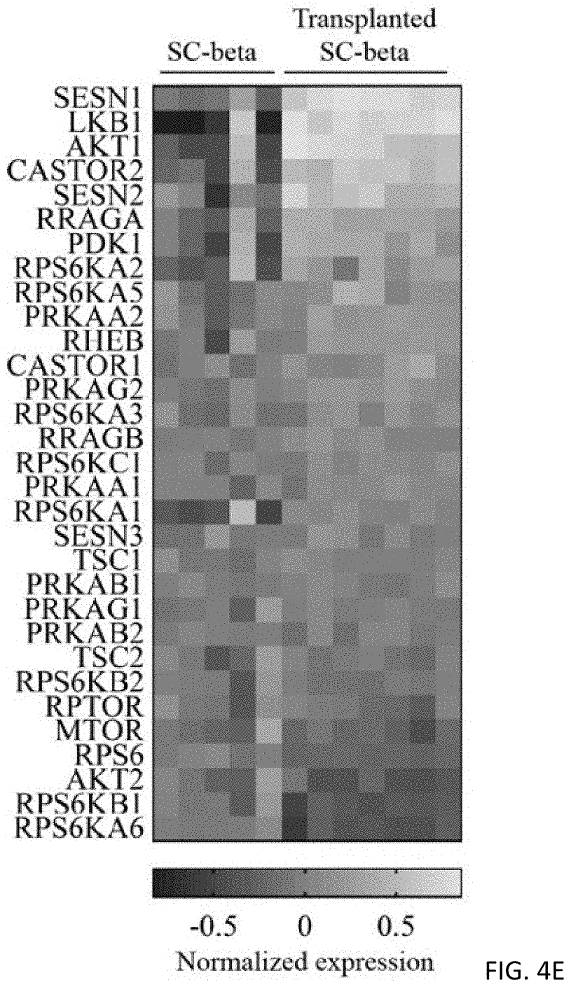

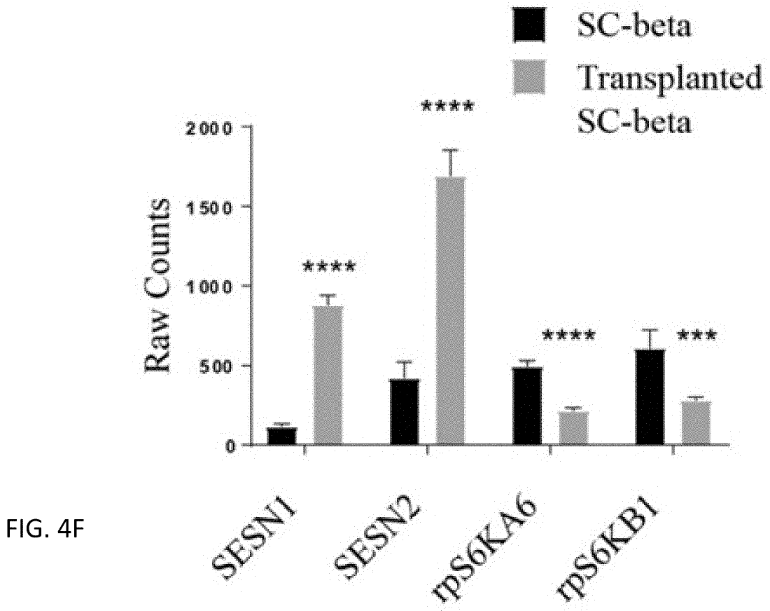

Next, which nutrients mTORC1 is specifically responsive to in SC-beta cells was tested. It was found that SC-beta cells respond strongly to amino acids, but do not increase mTORC1 activity in the presence of glucose, which was confirmed by FACS (FIGS. 4A and 4B) and immunostaining (FIG. 4C). This suggests that nutrient regulation of mTORC1 signaling in SC-beta cells is dominated by amino acids, and is comparable to fetal beta cells. Thus, the nutrient regulation of mTORC1 signaling in SC-beta cells was strongly activated by the amino acid-rich culture media. Overall, these results suggest that prolonged activation of mTORC1 by amino acids impairs glucose stimulated insulin secretion by SC-beta cells.

SC-Beta Cells Undergo a Shift from Immature to Mature Nutrient Responsiveness Upon Transplantation

Having insight into the nutrient sensitivity of mTORC1 in immature and mature beta cells, the next question was whether changing the environment of the SC-beta cells would change mTORC1 sensitivity to nutrients and its dynamics (FIG. 3A). SC-beta cells were collected from the final stage of the differentiation protocol and transplanted into mice for a period of two months, until reaching a stable functional state. Remarkably, after transplantation into mice, the nutrient sensitivity of mTORC1 in SC-beta cells significantly changes. During fasting or after injection of leucine into the recipient mouse, the mTORC1 pathway remains inactive. In contrast, feeding or injection of glucose strongly activates the mTORC1 pathway (FIG. 4D). These results demonstrate that changing the environment of the cells can shift the nutrient sensing characteristics of mTORC1 and imply that the dynamics of mTORC1 signaling in SC-beta cells also changes after transplantation.

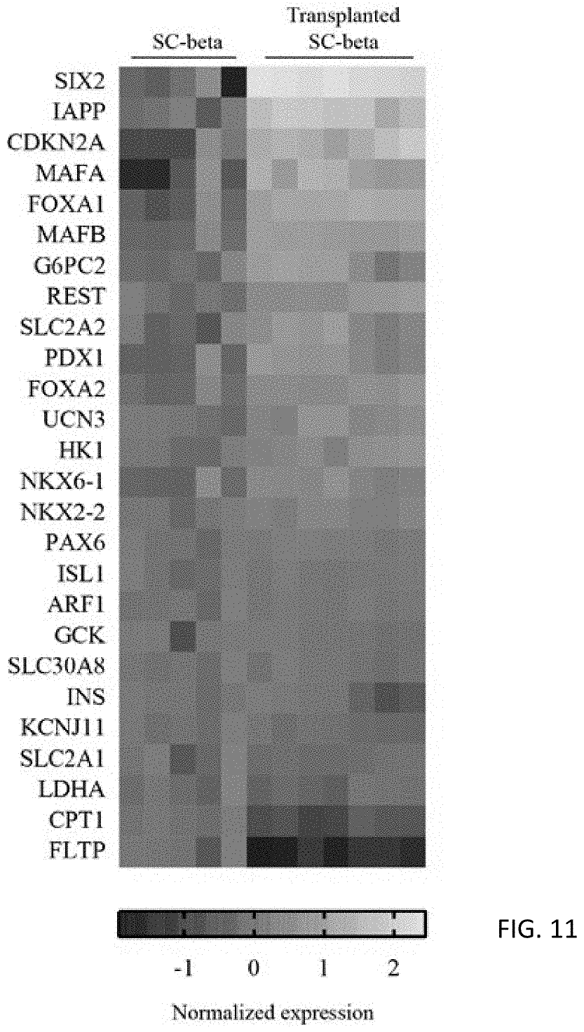

To find molecular drivers for the change in mTORC1 regulation by nutrients after transplantation, gene expression profiles of sorted SC-beta, collected before and after transplantation, were compared. Elevated expression of known maturation genes, such as SIX2, IAPP, MAFA, FOXA1, FOXA2 was found, and the senescence marker CDKN2A are also overexpressed after transplantation (Aguayo-Mazzucato et al., 2011; Artner et al., 2010; Gao et al., 2010; Heiman et al., 2016; Nishimura et al., 2015; Sun et al., 2010). Genes that have been reported as disallowed genes in beta cells such as CPT1 and LDHA (Pullen and Rutter, 2013) are downregulated upon transplantation (FIG. 11). Notably, genes that are responsible for nutrient sensing and inhibition of the mTORC1 pathway are upregulated after transplantation, including LKB1, the arginine sensor component CASTOR2 and the leucine sensors SESN1 and SESN2 (FIGS. 4E and 4F). The increased expression of the Sestrins is concomitant with the role of leucine in mTORC1 activation in beta cells (FIG. 8).

Amino Acid Sensing Control of mTORC1 Dynamics is Important for Glucose-Induced Insulin Secretion

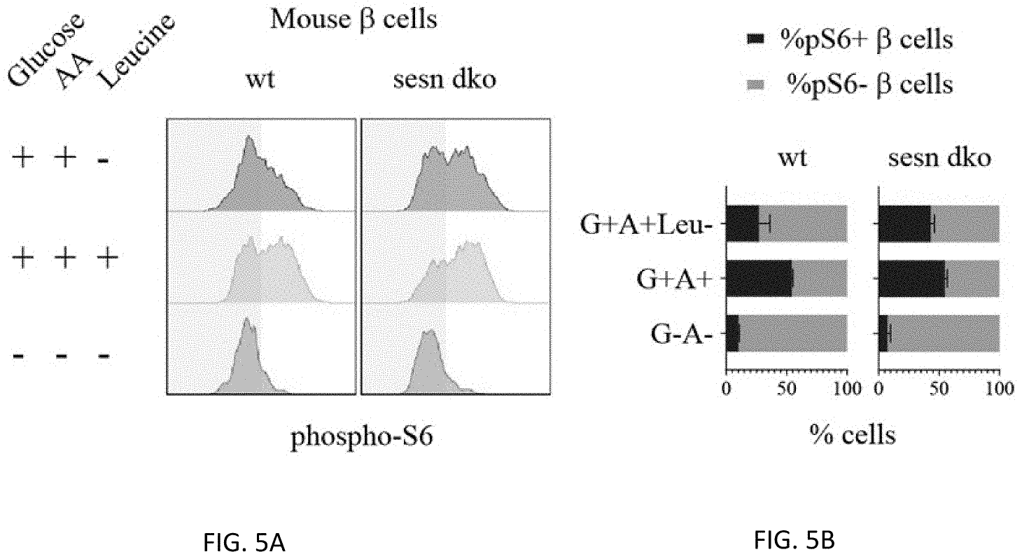

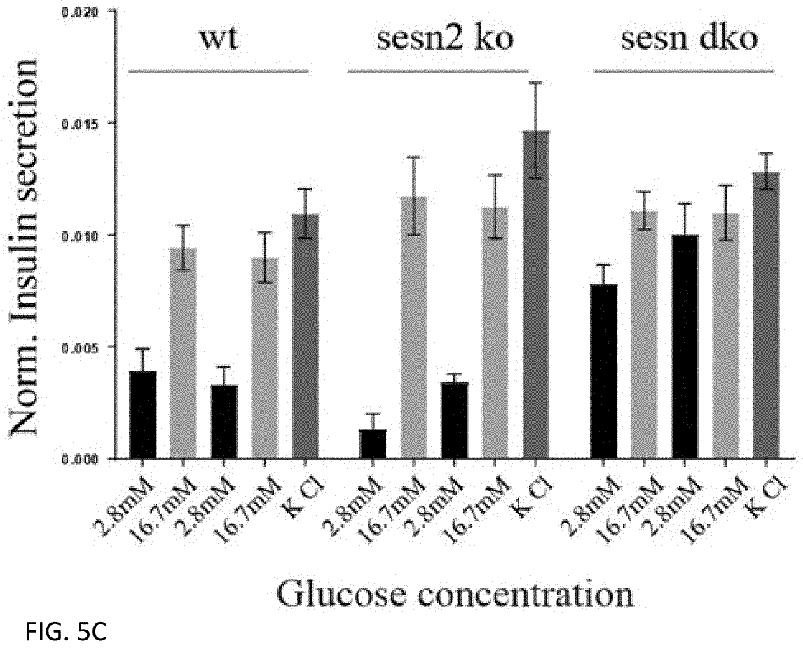

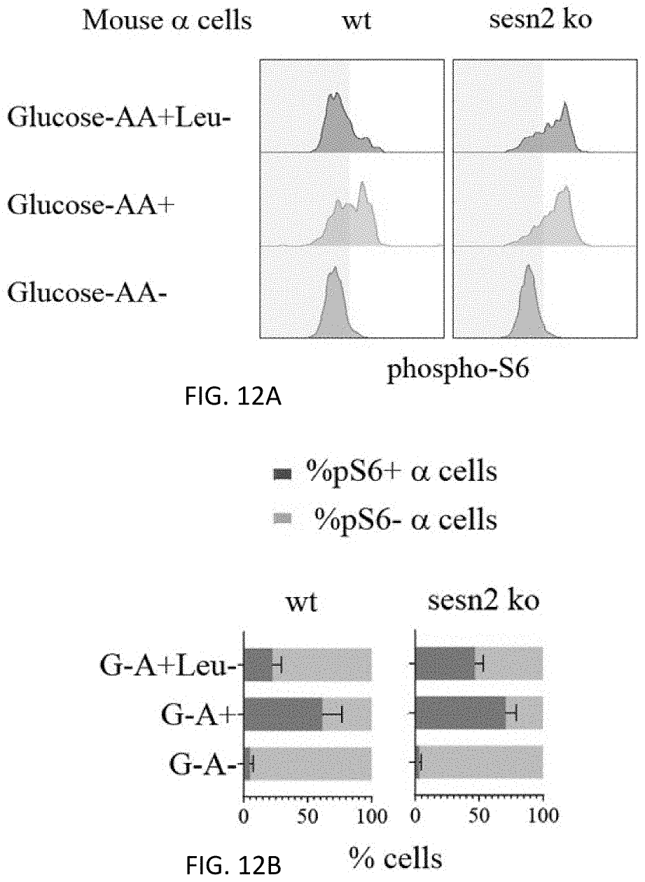

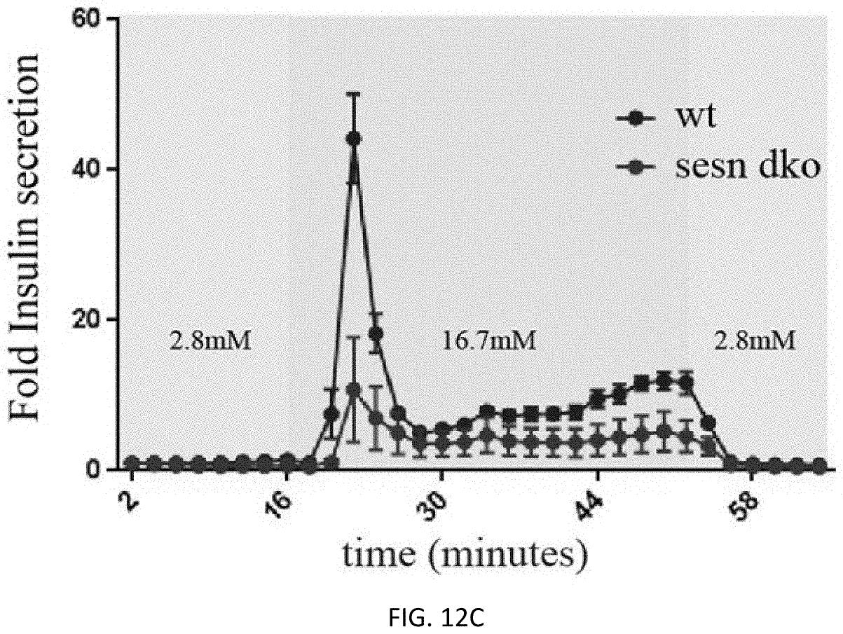

The switch from continuous to dynamic mTORC1 activity was tested to determine if the switch is controlled by the nutrient sensing pathway upstream of mTORC1. In addition, it was assessed whether this is important for regulated insulin secretion by beta cells. Amino acid sensing in mature beta cells was manipulated by generating mice in which the expression of SESN2 is globally deleted and SESN1 is specifically deleted in beta cells using inducible Cre driven by the insulin promoter. To test whether deleting Sestrins affected mTORC1 activation in response to amino acids in vivo, islets were isolated from the mutant mice and were incubated in different nutrient conditions. It was previously shown in cultured cells that leucine stimulation relieves the inhibition of Sestrins and allows mTORC1 activation. Similarly, it was found that in primary beta cells the absence of leucine inhibited mTORC1 signaling, whereas this inhibition was abolished in SESN-deficient beta cells (FIGS. 5A and 5B). In alpha cells, it was observed that removing SESN2 alone is sufficient to abolish the sensitivity to leucine starvation (FIG. 12). Furthermore, eliminating SESN1 and SESN2 expression in mature islets also affects their insulin secretion in response to glucose. Static and dynamic insulin secretion assays demonstrate elevated insulin secretion, particularly in response to basal glucose levels, by islets lacking SESN1 and SESN2, resembling immature beta cells (FIG. 5C and FIG. 12). These results suggest that amino acid sensing contributes to the regulation of mTORC1 signaling dynamics and is required to maintain proper function of mature beta cells.

Amino Acid Levels Control mTORC1 Dynamics and Response to Glucose in SC-Beta Cells

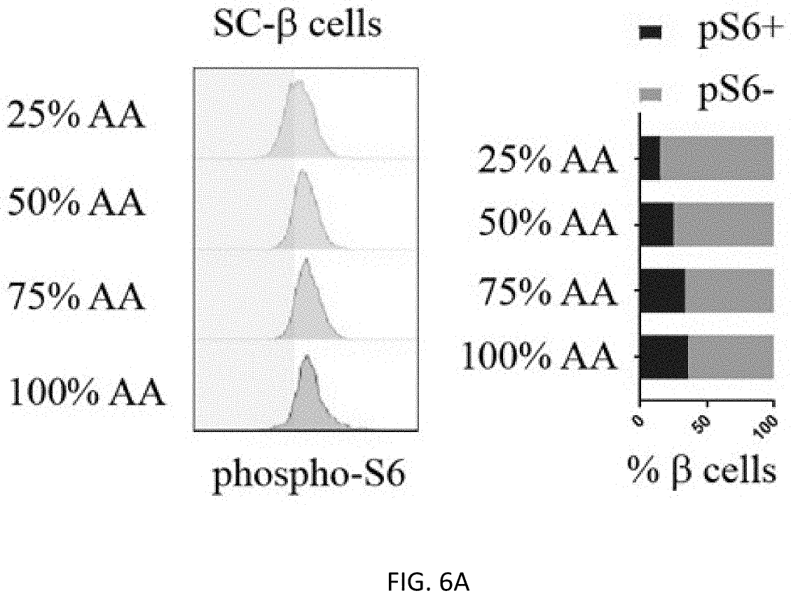

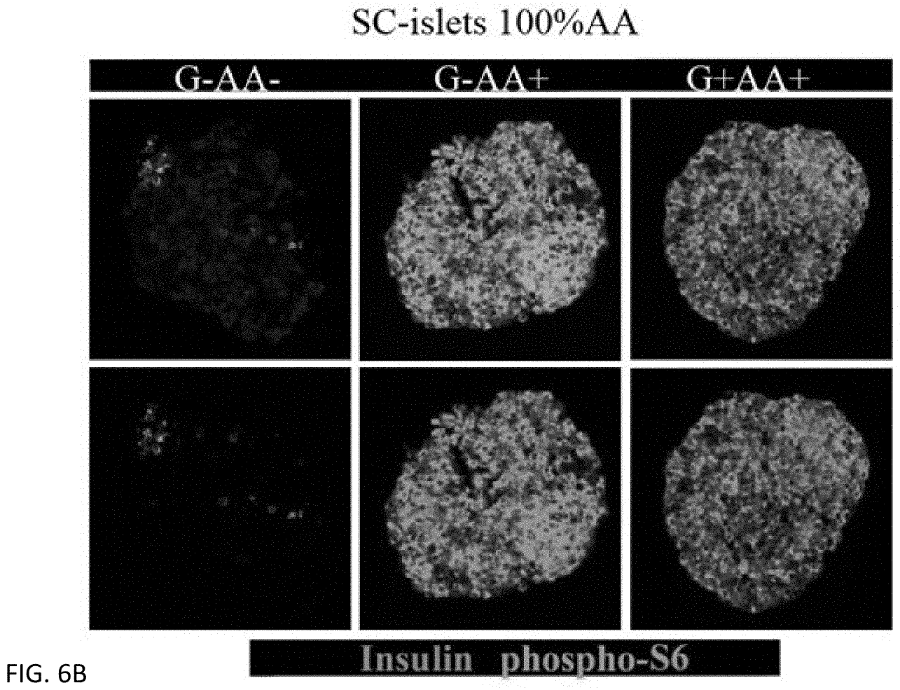

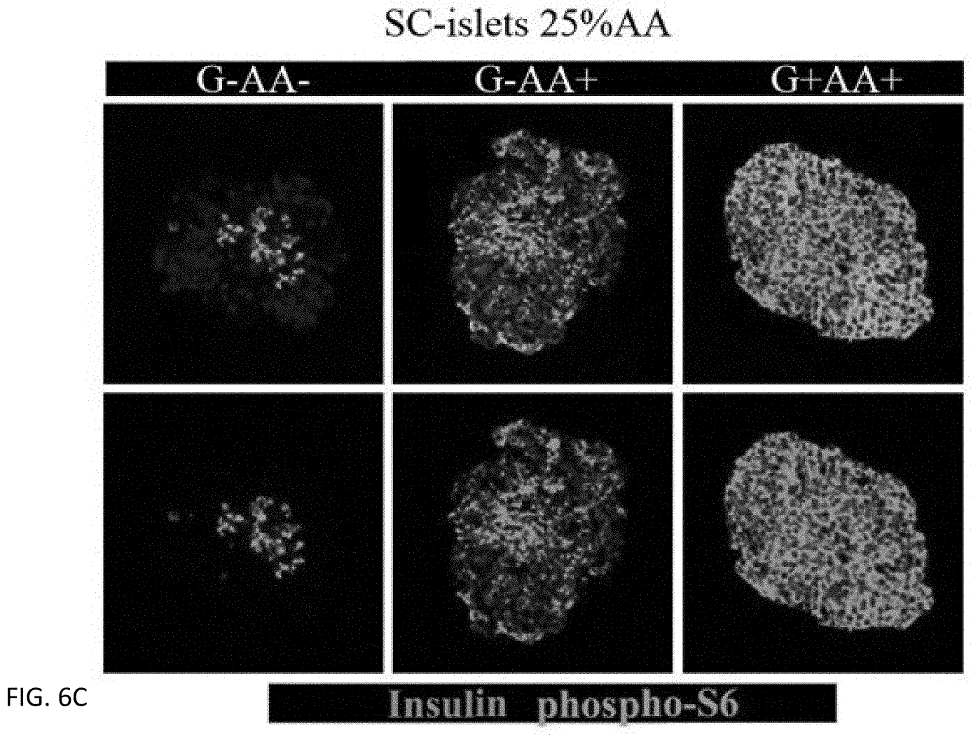

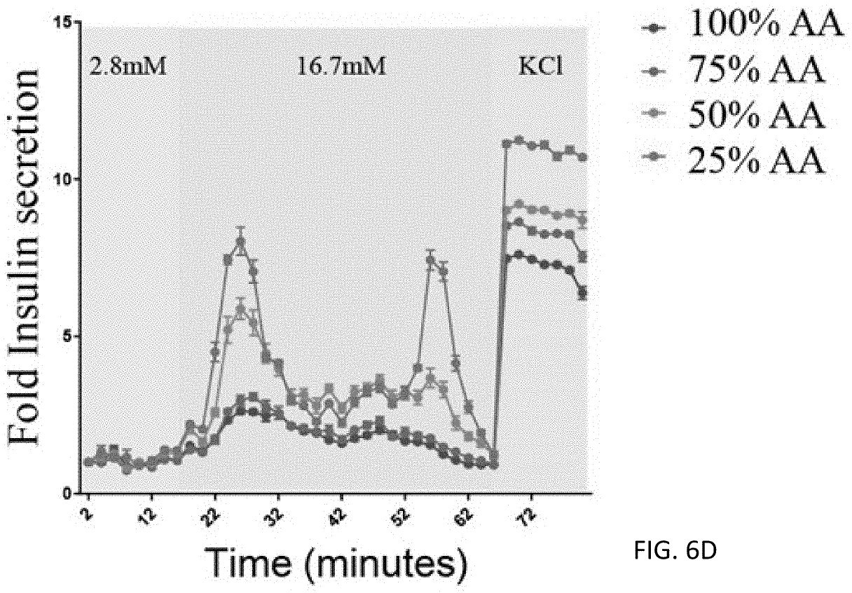

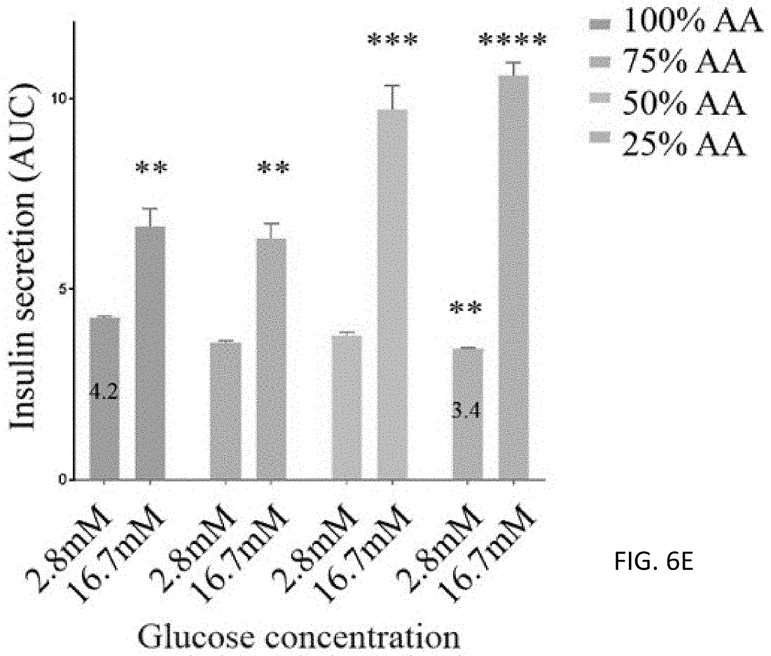

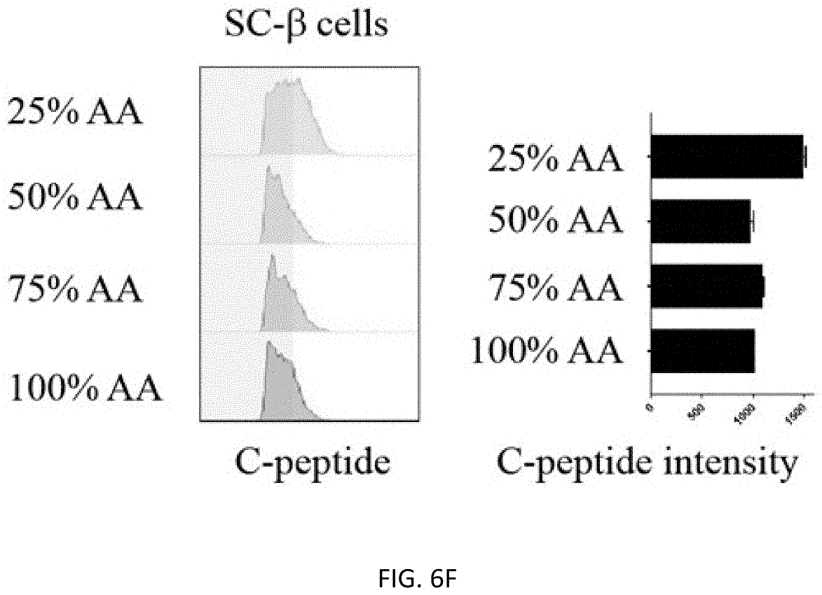

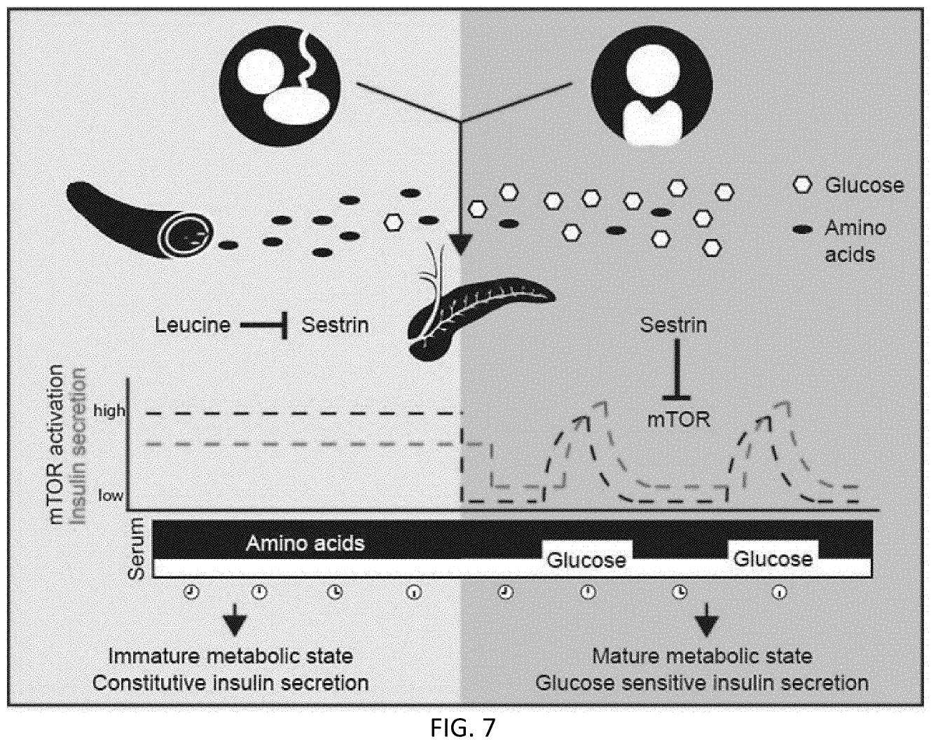

It was hypothesized that culturing amino acid responsive SC-beta cells in nutrient-rich media, with excessive amounts of amino acids, over-activates the mTORC1 pathway and thereby results in an immature insulin secretion. Therefore, it was sought to test whether recapitulating the postnatal change in nutrients and mTORC1 dynamics in vitro, by reducing amino acid levels in the culture media, might improve insulin secretion in response to glucose in SC-beta cells. SC-beta cell clusters were cultured for two weeks in media with varying amino acid concentrations. Cells grown in media with a lower concentration of amino acids display reduced levels of mTORC1 activity as measured by FACS staining for phosphorylated p-S6 (FIG. 6A). While SC-beta cells cultured in full amino acid levels were not dependent on glucose for full mTORC1 activity, cells cultured in 25% of the regular media amino acid levels suppressed mTORC1 signaling in the absence of glucose (FIGS. 6B and 6C), indicating that reduced amino acid conditions induce SC-beta cells to acquire a mature-like sensitivity of mTORC1 to glucose levels.

Next, it was assessed if the changes in mTORC1 nutrient sensitivity and activity induced by low amino acid conditions affect insulin secretion. To address this question, a dynamic GSIS assay was conducted on SC-islets that were grown in different concentrations of amino acids. It was found that culture in an amino acid-rich environment results in a weak insulin secretion response to high glucose. However, reducing amino acid levels to 75%, 50% or 25% of the regular media concentrations increased glucose-stimulated insulin secretion of the cells by 1.1, 1.6 and 2-fold, respectively (FIGS. 6D and 6E). Importantly, the effect of the enhanced stimulation is a combination of reduced insulin secretion in low glucose and increased insulin secretion in high glucose concentration. Remarkably, culturing SC-beta cells with 25% of media amino acid levels increases the average cellular insulin content as detected by FACS staining, perhaps because of the reduced basal insulin secretion and conservation of intracellular insulin (FIG. 6F). These results demonstrate that environmental conditions are sensed by SC-beta cells and influences their function in a manner that recapitulates the postnatal progression of beta cell maturation.

### Discussion

Newly-differentiated fetal beta cells continue to develop postnatally and acquire glucose induced insulin secretion in the first days after birth and beyond (Blum et al., 2012; Heiman et al., 2016; Stolovich-Rain et al., 2015). It is generally believed that postnatal maturation is a terminal step of a genetic differentiation program and transcriptional changes associated with this stage have identified molecular markers for beta cell maturation (Ni et al., 2017; Qiu et al., 2017). These findings show that in addition to genetic regulation there is a significant layer of metabolic control, by nutrients and nutrient sensing by mTORC1, on beta cell maturation.

Several previous studies have demonstrated a role for mTORC1 in beta cell function. Chronic pharmacological inhibition of mTORC1 using rapamycin found that mTORC1 positively regulates insulin secretion and beta cell replication. Concurrently, loss-of-function models of S6K1, rpS6, mTOR and Raptor exhibited hypoinsulinemia and glucose intolerance, with reduced beta cell size and function (Alejandro et al., 2017; Ardestani et al., 2018; Artner et al., 2010; Blandino-Rosano et al., 2017; Ni et al., 2017; Qiu et al., 2017; Ruvinsky et al., 2005; Saxton and Sabatini, 2017; Sinagoga et al., 2017; Sun et al., 2010; Swisa et al., 2017; Swisa et al., 2015; Wolfson and Sabatini, 2017; Wyant et al., 2017; Yu et al., 2018; Zeng et al., 2017). Also, stimulation of mTORC1 activity in beta cells by overexpression of Rheb or deletion of TSC or LKB1 increased beta cell mass and improved glucose tolerance (Alejandro et al., 2017; Blandino-Rosano et al., 2017; Mori et al., 2009; Swisa et al., 2015). On the other hand, chronically increased activity of the pathway was also correlated with beta cell dysfunction and diabetes (Ardestani et al., 2018). Although it is clear from these studies that the genetic components of the mTORC1 pathway are important for proper beta cell mass and function, it is not clear how the pathway is post-transcriptionally regulated in beta cells and how it affects beta cell function.

These findings demonstrate that the regulatory mechanisms controlling mTORC1 activity in beta cells are plastic, changing dramatically from the fetal to adult stage, which results in a neonatal switch from constitutive to periodic, feeding-regulated, mTORC1 activity. The regulation of mTORC1 is important for adjusting beta cell function to the developmental stage and the environment. Fetal development occurs under steady maternal supply of nutrients. In these conditions, stable secretion of insulin by beta cells is systematically required for cellular proliferation and growth. Hence, constitutive secretion of insulin by beta cells is important for proper embryonic development (Xu et al., 2004). The constitutive activation of mTORC1 confers a low glucose requirement for insulin secretion and hence continuous insulin release. At birth, a drastic change in nutrient availability has metabolic consequences that require an adaptation of beta cell function. The switch from the nutrient-rich environment in utero to intermittent postnatal feeding results in a greater dependence on insulin secretion by beta cells during feeding and suppressed release during fasting. Therefore, the functional shift of beta cells from constitutive to glucose-regulated insulin secretion after birth is vital to avoid hypoglycemia during fasting and to maximize nutrient absorption during feeding. The data strongly suggest that the change in nutrient sensitivity of mTORC1 signaling in beta cells after birth mediates this functional shift. The enhanced inhibition of mTORC1 by nutrient-sensitive regulators enables an increased glucose requirement for insulin secretion and thus reduces inappropriate release of insulin from mature beta cells during fasting.

It was also demonstrated that disrupting mTORC1 dynamics, in a loss-of-function model of SESN1 and SESN2 in mature mice, leads to improper beta cell function. These results also highlight the significance of amino acid sensing in the regulation of mTORC1 activity that had been well established in cell culture models, but whose physiologic significance has not been fully understood. Here it is shown that amino acid sensing by the Sestrins is important for proper regulation of mTORC1 activity and tissue function in vivo.

It is further demonstrated that beta cells sense the change in nutrient supply through the mTORC1 pathway. A complex machinery in mature beta cells keeps mTORC1 inactive in conditions unsuitable for growth. This regulatory network includes inhibitors such as TSC, AMPK and the Sestrins whose inhibition is released by growth factors, energy levels and amino acids, respectively. In the embryonic in utero environment, an abundance of nutrients, especially amino acids, constitutively releases these inhibitions and enables continuous mTORC1 activity. In the adult the availability of nutrients is periodic, leading to dynamic mTORC1 activity. Nevertheless, it was observed that the serum of the fetus contains very low levels of glucose, which raises the question of how mTORC1 is activated under these conditions. The data suggest that the answer lies in the sensitivity of the mTORC1 pathway to nutrients. The threshold for mTORC1 activation by amino acids is low in immature beta cells, perhaps due to reduced activity of inhibitory amino acid sensors, enabling full activity in the presence of amino acids only. Interestingly, a shift in mTORC1 sensitivity to amino acids, perhaps due to increased activity of amino acid sensors, leads to stronger inhibition of mTORC1 in the presence of amino acids only.

How do different cell types establish different sensitivities to nutrients to regulate mTORC1? One possibility is that different cells types can transport and metabolize different nutrients. In fact, single cell RNA sequencing during beta cell maturation revealed a maturation-associated reduction in genes involved in amino acid transport and metabolism (Zeng et al., 2017). The gene expression signature of elevated amino acid metabolism was correlated to proliferation and perhaps is mediated by the prolonged mTORC1 activity in immature beta cells. High basal insulin secretion due to a lack of a requirement for glucose is one specific challenge of SC-beta cell transplantation. Although SC-beta cells transplanted into mice undergo spontaneous functional maturation over time, the extended process can cause excessive insulin secretion that could lead to hypoglycemia and death. Increasing the glucose requirement for stimulation of insulin secretion is therefore critical for beta cell transplantation therapy. The fact that SC-beta cells, once transplanted into an in vivo environment, spontaneously reduce their basal insulin secretion points to a key role of environmental factors in the functional maturation of beta cells. Here it is reported that culturing SC-beta cells with reduced levels of amino acids, which more closely mimics the in vivo environment of mature beta cells and reduces mTORC1 activity, can increase the glucose requirement for insulin secretion and thus decrease basal insulin secretion in vitro. These functionally mature SC-beta cells will also provide a better platform for in vitro drug discovery and characterization of human metabolism and diabetes. It is suggested that a similar strategy of changing nutrient concentrations in media to induce functional maturation may be applied to the in vitro differentiation protocols of other stem cell-derived tissues, for transplantation therapy and for drug discovery.

In conclusion, it is shown that a shift in the sensitivity of mTORC1 to nutrients, which coincides with drastic nutritional changes at birth, alters mTORC1 signaling dynamics and leads to reduced basal insulin secretion and enhanced insulin secretion in response to high levels of glucose. This metabolic regulation of beta cell maturation, triggered by extrinsic changes in nutrient availability, is important for optimizing the function of beta cells for their environment.

### Materials and Methods

### Cell Culture

The hPSC line HUES8 (NIH human embryonic stem cell registry #0021) was used for all experiments. Undifferentiated HUES8 cells were maintained in supplemented mTeSR1 medium (StemCell Technologies) in 500 ml spinner flasks (Coming) set at a 70 rpm rotation rate in a 37° C. 5% CO2 incubator. Directed differentiation into SC-β cells was conducted as described previously with minor modifications. Briefly, 150 million cells were seeded in 300 ml mTeSR1 +10 μM ROCK Inhibitor Y27632, fed with mTeSR1 48 h later, and 72 h later stepwise differentiation stages were induced by the following treatments: DE stage: 24 h in S1 medium +100 ng/ml ActivinA +14 μg/ml CHIR99021, followed by 48 h in the same medium without CHIR99021. PGT stage: 72 h in S2 medium +50 ng/ml KGF. PP1 stage: 24 h in S3 medium +50 ng/ml KGF +0.25 μM Sant1 +2 μM Retinoic acid (RA) +500 nM PDBU +10 μM Y27632 +200 nM LDN193189, followed by 24 h in the same medium without LDN193189. PP2 stage: 6 days in S3 medium +50 ng/ml KGF +0.25 uM Sant1 +0.1 μM Retinoic acid (RA) +10 μM Y27632 +5 ng/ml ActivinA. EN stage: 4 days in BE5 medium +0.25 μM Sant1 +0.1 μM Retinoic acid (RA) +1 μM XXI +10 μM Alk5i II +1 μM T3 +20 ng/ml Betacellulin +10 μM Y27632, followed by 3 days in BE5 medium +25 nM Retinoic acid (RA) +1 μM XXI +10 μM Alk5i II +1 μM T3 +20 ng/ml Betacellulin +10 μM Y27632. SC-β stage: 14-20 days in supplemented CRML-1066 medium +10% defined fetal bovine serum (FBS, Hyclone) +10 μM Alk5i II +1 μM T3 (version 4), or in S3 medium (version 8). Primary adult islets from cadaveric donors (Prodo laboratories) were cultured overnight in supplemented CRML-1066 medium +10% FBS in low-attachment plates (Coming).

### Immunohistochemistry

Differentiated cell clusters or islets were fixed by immersion in 4% PFA for 1 hr at room temperature (RT). Samples were washed 3 times with PBS, embedded in Histogel (Thermo), and sectioned at 10 μm for histological analysis. Sections were subjected to deparrafinization using Histoclear (Thermoscientific; C78-2-G) and rehydrated. For antigen retrieval slides were emerged in 0.1M EDTA (Ambion; AM9261) and placed in a pressure cooker (Proteogenix; 2100 Retriever) for two hours.

Slides were blocked with PBS +0.1% Triton X-100 (VWR; EM-9400) +5% donkey serum (Jackson Immunoresearch; 017-000-121) for 1 hr at RT, followed by incubation in blocking solution with primary antibodies overnight at 4° C. The following primary antibodies were used: rat anti-insulin (pro-)/C-peptide (Developmental Studies Hybridoma Bank; GN-ID4, 1:300), mouse anti-glucagon (Santa Cruz; sc-514592 1:300), guinea pig anti-insulin (Dako; A0564 1:300), mouse anti-Nkx6.1 (University of Iowa, Developmental Hybridoma Bank; F55A12-supernatant) and rabbit anti phospho-S6 Ribosomal Protein (Ser240/244) (Cell Signaling; #5364, 1:100). Cells were washed twice in PBS the next day, followed by secondary antibody incubation for 2 hr at RT (protected from light). Secondary antibodies conjugated to Alexa Fluor 405,488, or 647 were used to visualize primary antibodies. Following two washes with PBS, the histology slides were mounted in Vectashield mounting medium (Vector Laboratories), covered with coverslips and sealed with nail polish. Representative images were taken using a Zeiss Axio Imager 2 microscope.

### Flow Cytometry

Differentiated cell clusters or islets were dispersed into single-cell suspension by incubation in TrypLE Express (Invitrogen) at 37° C. until clusters dissociated to single cells upon mixing by pipetting gently up and down (typically 10-15 min). Cells were spun down for 3 min at 1000 rpm, washed once in PBS (1 mL) and transferred to a 1.7 ml microcentrifuge tube (Bioscience; 11510). Cells were resuspended in perm/fix solution and incubated for 10 min (BD 554714). Cells were then washed once in perm/wash for 15 min (BD 554714).

Cells were then resuspended in perm/wash with primary antibodies and incubated at 4° C. overnight. Primary antibodies, diluted 1:300 unless otherwise noted: Mouse anti-Nkx6.1, rat anti-insulin (pro-)IC-peptide, mouse anti-glucagon, rabbit anti-phospho-S6 Ribosomal Protein (Ser240/244). Cells were washed twice in blocking buffer and then incubated in perm/wash buffer with secondary antibodies for 2 hr (protected from light). Secondary antibodies conjugated to Alexa Fluor 405, 488 or 647 (Life Technologies) were used to visualize primary antibodies. Cells were then washed 3 times in perm/wash solution and finally resuspended in 500-700 μl, filtered through a 40 μm nylon mash into flow cytometry tubes (BD Falcon; 352235), and analyzed using the LSR-II flow cytometer (BD Biosciences) with at least 30,000 events recorded. Analysis of the results was performed using FlowJo software.

### Gene Expression Analysis of Sorted Cells

To analyze global gene expression of SC-β cells, a recently described fixation and sorting strategy was used to isolate NKX6-1+/INS+ SC-β cells from the heterogenous cell clusters (Hrvatin et al., 2014). Two independent differentiation batches of SC-β clusters were harvested in single cell suspension using TrypLE and fixed in 4% PFA containing RNasin (VWR PAN2615) on ice for 30 min Fixed cells were incubated with primary antibodies (mouse anti-NKX6-1 diluted 1:100 and guinea pig anti-insulin diluted 1:100) for 30 min in buffer containing RNasin, washed twice and then incubated with secondary antibodies (anti-mouse Alexa Fluor 488 and anti-guinea pig Alexa Fluor 647) in buffer containing RNasin for 30 min each. After antibody staining, cells were sorted by fluorescence activated cell sorting (FACS) to obtain at least 100,000 cells per sample. Samples were subsequently incubated in Digestion Buffer (RecoverAll Total Nucleic Acid Isolation Kit, Ambion AM1975) at 50° C. for 3 hr, prior to RNA isolation according to manufacturer's instructions. RNA concentration was quantified using Nanodrop 1000. Double-stranded cDNA was generated by reverse transcription from at least 100 ng of total RNA according to manufacturer's instructions (Illumina TotalPrep RNA Amplification Kit, Life Technologies, AMIL1791). At least 750 ng cRNA per sample was hybridized to Human HT-12 Expression BeadChips (Illumina) using the Whole-Genome Expression Direct Hybridization kit (Illumina). Chips were scanned on the Illumina Beadstation 500. Raw data was adjusted by background subtraction and rank-invariant normalization (GenomeStudio software, Illumina). Before calculating fold change, an offset of 20 was added to all probe set means to eliminate negative signals. The p-values for differences between mean signals were calculated in GenomeStudio by t test and corrected for multiple hypotheses testing by the Benjamini-Hochberg method in combination with the Illumina custom false discovery rate (FDR) model. These SC-β cell microarray data and the previously published hPSC, PH, fetal β and adult β cell data (Hrvatin et al., 2014) were imported into the R statistical computing platform using the programming packages lumi and EMA. Samples were analyzed by hierarchical clustering using Pearson's correlation and Ward linkage. The pattern of clustering was robust to other distance and linkage metrics. Raw microarray data will be uploaded to publicly available databases.

### Mouse Transplantation Studies

All animal experiments were performed in accordance with Harvard University International Animal Care and Use Committee regulations. Cell transplantations into immunodeficient SCID-Beige mice (Jackson Laboratory) were conducted as described previously with minor modifications. SC-beta cell clusters were resuspended in RPMI-1640 medium (Life Technologies; 11875-093), aliquoted into PCR tubes and kept on ice until loading into a catheter for cell delivery under the mouse kidney capsule. Mice were anesthetized with 0.5 ml/25 g 1.25% Avertin/body weight, and the left ventricle kidney site was shaved and disinfected with betadine and alcohol. A 1 cm incision was made to expose the kidney, followed by insertion of the catheter needle and injection of the cell clusters. The abdominal cavity was closed with PDS absorbable sutures (POLY-DOX; 2016-06), and the skin was closed with surgical clips (Kent Scientific Corp; INS750346-2). Mice were placed on a 37° C. micro-temp circulating pump and blanket during the surgery/recovery period and given a 5 mg/kg carprofen dose post-surgery, re-applied 24 h after the initial dose. Wound clips were removed 14 days post-surgery and mice were monitored twice a week. To retrieve grafts, kidneys containing the grafts were dissected from freshly euthanized mice 4-6 weeks post-transplantation. For Immunohistochemistry, grafts were fixed in PBS +4% paraformaldehyde overnight, embedded in paraffin, and sectioned for histological analysis. Single-cell suspensions were washed with and resuspended in PBS, stained with TSQ at 37° C. for 10 min, filtered through a 40 μm nylon mash into flow cytometry tubes (BD Falcon), and TSQ+ cells were sorted using MoFlo flow cytometers (Beckman Coulter) into PBS +1% BSA (Sigma) on ice.

### Mice

Sesn mice were crossed with MIP-CreER mice {Wicksteed, 2010 #3259} (both on mixed B6 background). Two daily doses of 8 mg Tamoxifen (Sigma, 20 mg/ml in corn oil) were injected subcutaneously to 6-week old mice to obtain β-cell specific deletion of Sesn. All experiments were conducted using mouse litter batches, and where necessary experimental groups were comprised of multiple litters to allow statistical power. Two-sided Student's t-tests were used to compare mouse groups, under the assumption of normal distribution and observance of similar variance. Experiments using mice included both males and females. No statistical method was used to pre-determine sample size. The experiments were not randomized. There was no blinded allocation during experiments and outcome assessment. The ethics committee of Harvard university approved the study protocol for animal welfare.

### Ex Vivo Glucose Stimulated Insulin Secretion (GSIS)

Islets were isolated from whole pancreata using Collagenase P (Roche) injected to the pancreatic duct followed by Histopaque gradient (Sigma). Islets were incubated overnight in RPMI-1640 medium supplemented with 10% fetal bovine serum, L-glutamine and penicillin/streptomycin in a 37° C. 5% CO₂ incubator. Islets were handpicked and placed in basal Krebs buffer containing 2.8 mM glucose, and then transferred into Krebs solution containing 16.7 mM glucose. After 1 hour incubation at 37° C., islets were pelleted and supernatants were collected. The pellet was solubilized to assess intracellular insulin content. Insulin levels were measured by ELISA (Alpco). Values were normalized to insulin levels in the subsequently lysed islets, indicative of β-cell number in each sample. Islets from each individual mouse were assayed in triplicate. Normalization was done to β-cell number in each sample (pooled from 4 or more mice) scored by FACS following staining for insulin.

### Immunohistochemistry

Immunohistochemistry of kidney graft sections was performed as described previously (1). Sections were subjected to deparaffinization using Histoclear (Thermoscientific; C78-2-G) and rehydrated, them emerged in 0.1M EDTA (Ambion; AM9261) for antigen retrieval, and placed in a pressure cooker (Proteogenix; 2100 Retriever) for 2 h. Slides were blocked in PBS +0.1% Triton X-100 (VWR; EM-9400) +5% donkey serum (Jackson Immunoresearch; 017-000-121) for 1 h, followed by incubation in blocking solution with primary antibodies overnight at 4° C. The next day, cells were washed twice in PBS, followed by secondary antibody incubation for 2 h protected from light. For imaging, secondary antibodies were washed twice with PBS, and slides were mounted in Vectashield mounting medium with DAPI (Vector Laboratories; H-1200), covered with coverslips and sealed with nail polish. Images were taken using a Zeiss LSM 510 microscope.

### Tissue Section Staining and Analysis

Tissue processing for tissue section staining was performed as previously described {Nir, 2007 #3232}, using the following antibodies: insulin (DakoCytomation), Ki67 (NeoMarkers RM9106S0), Pdx1 (gift from Christopher Wright, Vanderbilt University), Nkx6.1 (β-cell Biology Consortium), Chga (Novus NB120-15160) human p16 (Abcam ab108349 and BD Pharmingen 551153), p21 (Santa Cruz Biotechnology sc-397), Lamp2a (Abcam ab18528), pS6 (Cell Signaling 5364), E-cadherin (BD Pharmingen 610182), Atp5a (Abcam ab14748), Cox17 (Novus NBP1-19696) and Ndufb (Sigma-Aldrich HPA005640). Secondary antibodies were from Jackson ImmunoResearch Laboratories. Fluorescent images were taken on a Nikon C1 confocal microscope at 400× magnification. To calculate cell size on tissue sections, the circumference of E-Cadherin-stained cells was determined using Image J Software. >100 cells from 6 mice were scored for each group. Values shown are for p16-positive β-cells in p16-induced mice, and for β-cells in control mice. To determine expression levels of p16, pS6 and mitochondrial markers in stained islet sections of human subjects NIS-Elements software was used to measure the mean intensity per section area of the fluorescent signal within islets, divided by the mean intensity per area of the signal in the surrounding acinar tissue in the same field, to account for differential staining background. For β-cell area determination, consecutive paraffin sections 50 μm apart, spanning the entire pancreas, were stained for insulin and hematoxylin. Digital 40× images were obtained, stitched together using NIS-Elements software, and the fraction of tissue stained for insulin was determined

### FACS Analysis

For flow cytometry, mouse islets were dissociated into a single-cell suspension with trypsin/EDTA treatment for 5 minutes at 37° C., followed by treatment with a cell fixation and permeabilization solution (BD Pharmingen). Stained cells were analyzed on a MACSQuant Analyzer (DAKO). In all analyses cells were pooled from at least 3 mice in each group. Live human islets were obtained from pancreata of brain-dead patients as previously described {O'Gorman, 2010 #3353}, under approval of the Health Research Ethics Board of the University of Alberta and following patient informed consent. Patient details are presented in Supplementary Table 2. Several hundreds of islets were obtained from each subject, and were dissociated prior to staining. Antibody staining was performed using standard procedures, the antibodies mentioned above, as well as: mouse p16 (Santa Cruz Biotechnology sc-1207), Lamp2a (Abcam ab18528).

### Expression Profiling

GFP-positive cells were isolated by FACS from dissociated islets of Insulin-rtTA/tet-GFP/tet-p16, and control Insulin-rtTA/Tet-GFP mice, following tet treatment for 10 days. Total RNA was isolated by TRIzol (Invitrogen) extraction followed by RNeasy Plus Micro Kit (Qiagen) from ˜50,000 β-cells, from 2 control and 3 p16-expressing mice. Libraries were prepared and sequenced using Illumina's directional RNA sequencing protocol (Hi Seq). Reads were mapped using TopHat2 and quantification and normalization were done using Cuffdiff to produce gene-level normalized expression values (FPKMs). Up- and downregulated genes with P<0.05 were tested for enrichment for gene sets using the hypergeometric method, FDR<0.05. Gene sets were derived from MSigDB or KEGG or compiled from the literature as described in Supplementary Table 1.

Human Islet mRNA

Adult human islets for the quantitative RT-PCR were obtained from Integrated Islet Distribution program (iidp.coh.org/), and studied as described {Dai, 2012 #3349}. Adult human islets were from 4 female and 5 male donors (age 44.7+4.2 years [range, 20-60], BMI 25.02+0.84 kg/m2 [range 21.2.-29.1]. The cold ischemia time before pancreas isolation was 12.18+2.48 h. Nine normal juvenile pancreata were used in this study (6 months old, n=1; 14 months, n=1; 20 months, n=1; 3 years, n=2; 4 years, n=1; 5 years, n=2; 9 years, n=1) via protocol with the National Disease Research Interchange and International Institute for the Advancement of Medicine (IIAM). The islets were isolated as previously described {Walsh, 2012 #3350}. De-identified human islet studies were approved by the Vanderbilt Institutional Review Board. Extraction of total RNA from human islets and performance of qRT-PCR were done as previously described {Dai, 2012 #3349}, using the TaqMan primer-probe (CDKN2A, Hs00923894_m1) and reagents from Applied Biosystems (Foster city, Calif.). ACTB (Hs99999903_m1) was used as endogenous control.

### REFERENCES

Aguayo-Mazzucato, C., Koh, A., El Khattabi, I., Li, W. C., Toschi, E., Jermendy, A., Juhl, K., Mao, K., Weir, G. C., Sharma, A., et al. (2011). Mafa expression enhances glucose-responsive insulin secretion in neonatal rat beta cells. Diabetologia 54, 583-593.
Alejandro, E. U., Bozadjieva, N., Blandino-Rosano, M., Wasan, M. A., Elghazi, L., Vadrevu, S., Satin, L., and Bernal-Mizrachi, E. (2017). Overexpression of Kinase-Dead mTOR Impairs Glucose Homeostasis by Regulating Insulin Secretion and Not beta-Cell Mass. Diabetes 66, 2150-2162.
Arda, H. E., Li, L., Tsai, J., Torre, E. A., Rosli, Y., Peiris, H., Spitale, R. C., Dai, C., Gu, X., Qu, K., et al. (2016). Age-Dependent Pancreatic Gene Regulation Reveals Mechanisms Governing Human beta Cell Function. Cell Metab 23, 909-920.

Ardestani, A., Lupse, B., Kido, Y., Leibowitz, G., and Maedler, K. (2018). mTORC1 Signaling: A Double-Edged Sword in Diabetic beta Cells. Cell Metab 27, 314-331.
Artner, I., Hang, Y., Mazur, M., Yamamoto, T., Guo, M., Lindner, J., Magnuson, M. A., and Stein, R. (2010). MafA and MafB regulate genes critical to beta-cells in a unique temporal manner. Diabetes 59, 2530-2539.
Bader, E., Migliorini, A., Gegg, M., Moruzzi, N., Gerdes, J., Roscioni, S. S., Bakhti, M., Brandl, E., Irmler, M., Beckers, J., et al. (2016). Identification of proliferative and mature beta-cells in the islets of Langerhans. Nature 535, 430-434.
Blandino-Rosano, M., Barbaresso, R., Jimenez-Palomares, M., Bozadjieva, N., Werneck-de-Castro, J. P., Hatanaka, M., Mirmira, R. G., Sonenberg, N., Liu, M., Ruegg, M. A., et al. (2017). Loss of mTORC1 signaling impairs beta-cell homeostasis and insulin processing. Nat Commun 8, 16014.
Blum, B., Hrvatin, S., Schuetz, C., Bonal, C., Rezania, A., and Melton, D. A. (2012). Functional beta-cell maturation is marked by an increased glucose threshold and by expression of urocortin 3. Nat Biotechnol 30, 261-264.
Gao, N., Le Lay, J., Qin, W., Doliba, N., Schug, J., Fox, A. J., Smirnova, O., Matschinsky, F. M., and Kaestner, K. H. (2010). Foxa1 and Foxa2 maintain the metabolic and secretory features of the mature beta-cell. Mol Endocrinol 24, 1594-1604.
Helman, A., Klochendler, A., Azazmeh, N., Gabai, Y., Horwitz, E., Anzi, S., Swisa, A., Condiotti, R., Granit, R. Z., Nevo, Y., et al. (2016). p16(Ink4a)-induced senescence of pancreatic beta cells enhances insulin secretion. Nat Med 22, 412-420.
Henquin, J. C., and Nenquin, M. (2018). Immaturity of insulin secretion by pancreatic islets isolated from one human neonate. J Diabetes Investig 9, 270-273.
Hrvatin, S., O'Donnell, C. W., Deng, F., Millman, J. R., Pagliuca, F. W., Dilorio, P., Rezania, A., Gifford, D. K., and Melton, D. A. (2014). Differentiated human stem cells resemble fetal, not adult, beta cells. Proc Natl Acad Sci USA 111, 3038-3043.
Mori, H., Inoki, K., Opland, D., Munzberg, H., Villanueva, E. C., Faouzi, M., Ikenoue, T., Kwiatkowski, D. J., Macdougald, O. A., Myers, M. G., Jr., et al. (2009). Critical roles for the TSC-mTOR pathway in beta-cell function. Am J Physiol Endocrinol Metab 297, E1013-1022.
Morton, S. U., and Brodsky, D. (2016). Fetal Physiology and the Transition to Extrauterine Life. Clin Perinatol 43, 395-407.
Ni, Q., Gu, Y., Xie, Y., Yin, Q., Zhang, H., Nie, A., Li, W., Wang, Y., Ning, G., Wang, W., et al. (2017). Raptor regulates functional maturation of murine beta cells. Nat Commun 8, 15755.
Nishimura, W., Takahashi, S., and Yasuda, K. (2015). MafA is critical for maintenance of the mature beta cell phenotype in mice. Diabetologia 58, 566-574.
Pagliuca, F. W., Millman, J. R., Gurtler, M., Segel, M., Van Dervort, A., Ryu, J. H., Peterson, Q. P., Greiner, D., and Melton, D. A. (2014). Generation of functional human pancreatic beta cells in vitro. Cell 159, 428-439.
Pan, F. C., and Wright, C. (2011). Pancreas organogenesis: from bud to plexus to gland. Dev Dyn 240, 530-565.
Pullen, T. J., and Rutter, G. A. (2013). When less is more: the forbidden fruits of gene repression in the adult beta-cell. Diabetes Obes Metab 15, 503-512.
Qiu, W. L., Zhang, Y. W., Feng, Y., Li, L. C., Yang, L., and Xu, C. R. (2017). Deciphering Pancreatic Islet beta Cell and alpha Cell Maturation Pathways and Characteristic Features at the Single-Cell Level. Cell Metab 25, 1194-1205 e1194.
Robinton, D. A., and Daley, G. Q. (2012). The promise of induced pluripotent stem cells in research and therapy. Nature 481, 295-305.
Rozzo, A., Meneghel-Rozzo, T., Delakorda, S. L., Yang, S. B., and Rupnik, M. (2009). Exocytosis of insulin: in vivo maturation of mouse endocrine pancreas. Ann N Y Acad Sci 1152, 53-62.
Ruvinsky, I., Sharon, N., Lerer, T., Cohen, H., Stolovich-Rain, M., Nir, T., Dor, Y., Zisman, P., and Meyuhas, O. (2005). Ribosomal protein S6 phosphorylation is a determinant of cell size and glucose homeostasis. Genes Dev 19, 2199-2211.
Sancak, Y., Bar-Peled, L., Zoncu, R., Markhard, A. L., Nada, S., and Sabatini, D. M. (2010). Ragulator-Rag complex targets mTORC1 to the lysosomal surface and is necessary for its activation by amino acids. Cell 141, 290-303.
Sancak, Y., Peterson, T. R., Shaul, Y. D., Lindquist, R. A., Thoreen, C. C., Bar-Peled, L., and Sabatini, D. M. (2008). The Rag GTPases bind raptor and mediate amino acid signaling to mTORC1. Science 320, 1496-1501.
Saxton, R. A., and Sabatini, D. M. (2017). mTOR Signaling in Growth, Metabolism, and Disease. Cell 168, 960-976.
Sinagoga, K. L., Stone, W. J., Schiesser, J. V., Schweitzer, J. I., Sampson, L., Zheng, Y., and Wells, J. M. (2017). Distinct roles for the mTOR pathway in postnatal morphogenesis, maturation and function of pancreatic islets. Development 144, 2402-2414.
Slack, J. M. (1995). Developmental biology of the pancreas. Development 121, 1569-1580.
Sneddon, J. B., Tang, Q., Stock, P., Bluestone, J. A., Roy, S., Desai, T., and Hebrok, M. (2018). Stem Cell Therapies for Treating Diabetes: Progress and Remaining Challenges. Cell Stem Cell 22, 810-823.
Stolovich-Rain, M., Enk, J., Vikesa, J., Nielsen, F. C., Saada, A., Glaser, B., and Dor, Y. (2015). Weaning triggers a maturation step of pancreatic beta cells. Dev Cell 32, 535-545.
Sun, G., Tarasov, A. I., McGinty, J. A., French, P. M., McDonald, A., Leclerc, I., and Rutter, G. A. (2010). LKB1 deletion with the RIP2.Cre transgene modifies pancreatic beta-cell morphology and enhances insulin secretion in vivo. Am J Physiol Endocrinol Metab 298, E1261-1273.
Swisa, A., Avrahami, D., Eden, N., Zhang, J., Feleke, E., Dahan, T., Cohen-Tayar, Y., Stolovich-Rain, M., Kaestner, K. H., Glaser, B., et al. (2017). PAX6 maintains beta cell identity by repressing genes of alternative islet cell types. J Clin Invest 127, 230-243.
Swisa, A., Granot, Z., Tamarina, N., Sayers, S., Bardeesy, N., Philipson, L., Hodson, D. J., Wikstrom, J. D., Rutter, G. A., Leibowitz, G., et al. (2015). Loss of Liver Kinase B1 (LKB1) in Beta Cells Enhances Glucose-stimulated Insulin Secretion Despite Profound Mitochondrial Defects. J Biol Chem 290, 20934-20946.
Ward Platt, M., and Deshpande, S. (2005). Metabolic adaptation at birth. Semin Fetal Neonatal Med 10, 341-350.
Wolfson, R. L., Chantranupong, L., Saxton, R. A., Shen, K., Scaria, S. M., Cantor, J. R., and Sabatini, D. M. (2016). Sestrin2 is a leucine sensor for the mTORC1 pathway. Science 351, 43-48.
Wolfson, R. L., and Sabatini, D. M. (2017). The Dawn of the Age of Amino Acid Sensors for the mTORC1 Pathway. Cell Metab 26, 301-309.
Wyant, G. A., Abu-Remaileh, M., Wolfson, R. L., Chen, W. W., Freinkman, E., Danai, L. V., Vander Heiden, M. G., and Sabatini, D. M. (2017). mTORC1 Activator SLC38A9 Is Required to Efflux Essential Amino Acids from Lysosomes and Use Protein as a Nutrient. Cell 171, 642-654 e612.
Xu, Q. G., Li, X. Q., Kotecha, S. A., Cheng, C., Sun, H. S., and Zochodne, D. W. (2004). Insulin as an in vivo growth factor. Exp Neurol 188, 43-51.
Yu, F., Wei, R., Yang, J., Liu, J., Yang, K., Wang, H., Mu, Y., and Hong, T. (2018). FoxO1 inhibition promotes differentiation of human embryonic stem cells into insulin producing cells. Exp Cell Res 362, 227-234.
Zeng, C., Mulas, F., Sui, Y., Guan, T., Miller, N., Tan, Y., Liu, F., Jin, W., Carrano, A. C., Huising, M. O., et al. (2017). Pseudotemporal Ordering of Single Cells Reveals Metabolic Control of Postnatal beta Cell Proliferation. Cell Metab 25, 1160-1175 e1111.

What is claimed is:

1. A method of producing a mature SC-β cell comprising contacting an immature β cell with an mTOR inhibitor, thereby producing a mature SC-β cell.

2. The method of claim 1, wherein the mTOR inhibitor is an inhibitor of both mTORC1 and mTORC2.

3. The method of claim 1, wherein the mTOR inhibitor inhibits phosphorylation of Ribosomal protein S6.

4. The method of claim 1, wherein the mTOR inhibitor inhibits both phosphorylation of Ribosomal protein S6 and 4E-BP1.

5. The method of claim 1, wherein the mTOR inhibitor is selected from the group consisting of rapamycin, Torin1, Torin2, everolimus and temsirolimus.

6. The method of claim 1, wherein the mTOR inhibitor is Torin1.

7. The method of claim 1, wherein the immature β cell is derived from an iPS cell.

8. The method of claim 1, wherein the immature β cell is derived from an ES cell.

10. The method of claim 1, wherein the immature β cell is derived from a fibroblast.

11. The method of claim 1, wherein the mature SC-β cell exhibits increased GSIS response as compared to an immature β cell.

12. The method of claim 1, wherein the immature β cell is contacted with the mTOR inhibitor during a final stage of a differentiation protocol.

13. The method of claim 1, wherein the immature β cell is contacted with the mTOR inhibitor for a period of 1 to 3 days.

14. A method of producing a mature SC-β cell comprising culturing an immature β cell in a nutrient poor culture medium, thereby producing a mature SC-β cell.

15. The method of claim 14, wherein the nutrient poor culture medium comprises a reduced level of amino acids as compared to a non-responsive culture medium comprising 100% amino acids.

16. The method of claim 14, wherein the nutrient poor culture medium comprises 75% amino acid levels as compared to the non-responsive culture medium.

17. The method of claim 16, wherein the SC-β cells cultured in a culture medium comprising 75% amino acid levels exhibit at least a 1.1 fold increase in GSIS response.

18. The method of claim 14, wherein the nutrient poor culture medium comprises 50% amino acid levels as compared to the non-responsive culture medium.

19. The method of claim 18, wherein the SC-β cells cultured in a culture medium comprising 50% amino acid levels exhibit at least a 1.6 fold increase in GSIS response.

20. The method of claim 14, wherein the nutrient poor culture medium comprises 25% amino acid levels as compared to the non-responsive culture medium.

21. The method of claim 20, wherein the SC-β cells cultured in a culture medium comprising 25% amino acid levels exhibit a 2 fold increase in GSIS response.

22. The method of claim 14, wherein the immature β cell is derived from an iPS cell.

23. The method of claim 14, wherein the immature β cell is derived from an ES cell.

24. The method of claim 14, wherein the immature β cell is derived from a fibroblast.
